(19) United States
(12) Patent Application Publication
(10) Pub. No.: US 2025/0213775 A1
(43) Pub. Date: Jul. 3, 2025

(54) TISSUE REMOVAL SYSTEMS AND METHODS

(71) Applicant: CLARIA MEDICAL, INC., Mountain View, CA (US)

(72) Inventors: Daniel E. FRANCIS, Mountain View, CA (US); Alexey SALAMINI, San Francisco, CA (US); Joseph N. JONES, Boise, FL (US); Kevin LE, Palo Alto, CA (US); Jeremy C. KOEHLER, San Mateo, CA (US); Scott C. ANDERSON, Felton, CA (US); Ronald G. FRENCH, San Jose, CA (US); Steven W. KIM, Los Altos, CA (US); Will SEGALLA, Mountain View, CA (US)

(21) Appl. No.: 18/848,871

(22) PCT Filed: Mar. 23, 2023
(86) PCT No.: PCT/US2023/016158

§ 371 Date: Sep. 19, 2024

Related U.S. Application Data
(60) Provisional application No. 63/358,050, filed on Jul. 1, 2022.
(60) Provisional application No. 63/350,319, filed on Jun. 8, 2022.
(60) Provisional application No. 63/332,623, filed on Apr. 19, 2022.
(60) Provisional application No. 63/325,116, filed on Mar. 29, 2022.
(60) Provisional application No. 63/323,977, filed on Mar. 25, 2022.

(51) Int. Cl. A61M 1/00

(52) U.S. Cl. CPC A61M 1/87

(57) ABSTRACT

Components, systems and kits for capturing and/or removing tissue from mammalian bodies may include a tissue container that may be introduced into a body cavity within which a tissue specimen may be placed, cut and removed from the body cavity. Methods of using these components, systems and kits are also described.

The present application is a national stage application under 35 U.S.C. section 371, which claims the benefit of priority to PCT Application No. PCT/US2023/016158, filed Mar. 23, 2023, by Daniel E. FRANCIS et al., and titled “TISSUE REMOVAL SYSTEMS AND METHODS,” which claims the benefit of U.S. Provisional Application No. 63/323,977 filed Mar. 25, 2022, entitled “Tissue Removal Systems and Methods,” U.S. Provisional Application No. 63/325,116, filed Mar. 29, 2022, entitled “Tissue Removal Systems and Methods,” U.S. Provisional Application No. 63/332,623, filed Apr. 19, 2022, entitled “Tissue Removal Systems and Methods,” U.S. Provisional Application No. 63/350,319, filed Jun. 8, 2022, entitled “Tissue Removal Systems and Methods,” and U.S. Provisional Application No. 63/358,050, filed Jul. 1, 2022, entitled “Tissue Removal Systems and Methods,” each of which is incorporated by reference herein in its entirety.

The present application is related to international patent application no. PCT/US2017/029162 filed Apr. 24, 2017 entitled “Systems and Methods for Tissue Capture and Removal,” international patent application no. PCT/US2018/056915 filed Oct. 22, 2018 entitled “Systems and Methods for Tissue Capture and Removal,” and international patent application no. PCT/US20/46135 filed Aug. 13, 2020 entitled “Tissue Removal Systems and Methods,” the contents of each of which are incorporated by reference herein in their entirety.

### BACKGROUND

In the field of health care in human and veterinary medicine, it is often desirable or even necessary to remove tissue from a patient's body. Such tissue, typically in the form of mass, tumor, or organ, some of which may be benign, cancerous, pre-cancerous, or be suspected of being cancerous or pre-cancerous, may be removed via traditional surgical techniques, including open surgery as well as minimally invasive approaches.

Among minimally invasive approaches, laparoscopic procedures in which a tissue specimen is removed via a small incision using specialized tools are well known. Minimally invasive procedures such as laparoscopy and mini-laparotomy may also employ the use of tools operated robotically. Procedures performed via a minimally invasive approach include those performed in the abdominal, pelvic and thoracic cavities. Cholecystectomies, nephrectomies, colectomies, hysterectomies, myomectomies, oophorectomies, and other procedures in gastrointestinal, gynecological and urological categories are common as are minimally invasive arthroscopy, cystoscopy, and thoracoscopy procedures. Various advantages cited with regard to minimally invasive procedures include enhanced safety, reduced pain, lower risk of infection, shorter recovery times, shorter hospital stays, increased patient satisfaction, and lower cost, among others.

Often, the tissue specimen to be removed via minimally invasive procedures is larger than the incisions used to gain access to the tissue specimen. As such, techniques have been developed to safely remove such specimens while maintaining the advantages of a minimally invasive approach. One such technique is morcellation, in which the tissue specimen is cut or processed into pieces while still inside the patient, or at the level of the skin, or just outside the patient, so that they may be more readily removed. The earliest form of morcellation involved scissors or scalpels to chop up a uterus during vaginal hysterectomy, so that the specimen could be removed through the vagina. Similar manual cutting techniques may be employed when removing many types of tumors or organs through an incision in the abdomen. Later, electromechanical power morcellators were developed that could be deployed via laparoscopic ports which enabled the tissue fragments to be removed via the ports.

In the field of gynecology, the hysterectomy is a common procedure that is performed in approximately 500,000 women per year in the United States alone. The hysterectomy procedure involves removing a woman's uterus, which may be necessary due to any of a variety of reasons, the most common of which (greater than 50% of cases in the U.S.) is due to the presence of uterine fibroids. Uterine fibroids (also known as leiomyoma) are benign tumors which tend to enlarge the specimen, often to the point of where the specimen cannot fit out via the vaginal orifice or minimally invasive surgical incision without the benefit of some form of morcellation. Such hysterectomies may be performed via traditional open surgical techniques or minimally invasive techniques, such as laparoscopy with the use of morcellation or bulk tissue reduction. Hysterectomies may be partial, e.g., involving removal of only the uterus, or total, in which the uterus and uterine cervix are both removed. In either case, the ovaries and/or the fallopian tubes may or may not simultaneously be removed.

For years, power morcellation has been used in gynecologic surgery to remove large uteri from patients via small holes, as is necessary in minimally invasive surgery. The most common application of power morcellation in gynecologic surgery has typically involved morcellating a large, fibroid uterus to remove it from a patient's body during a hysterectomy or laparoscopic procedure, although there are a number of other applications as well-notably a myomectomy in which the uterine fibroids are removed, but the uterus itself is preserved within the patient's body in case the patient desires future fertility.

Since hysterectomies involving an enlarged uterus are very common, and since minimally invasive surgery offers many benefits to the patient, surgeon, hospital, and payer, the use of power morcellation has become commonplace. However, the potential for occult cancers hidden within the uterus that cannot be detected preoperatively and that could potentially be spread around the patient's body with grave consequences during morcellation has been a source of concern. As such, even though most hysterectomies are associated with uteri that do not involve any actual or suspected cancer, traditional open surgery, with its added risk, complication rates, longer hospitalizations, more difficult recoveries, etc., is prevalent. Therefore, techniques and systems are desirable that afford safe removal and processing of tissue specimens, even in the possible presence of an occult malignancy.

Clinically there is a strong need for a tissue containment and extraction system and a container that holds this tissue in a breach-resistant manner. There are many benefits of an easy-to-use, breach-resistant system. First, it can prevent the spread of malignant cells of the contained tissue sample. Second, it can prevent iatrogenic injury of important organs and structures that need to be protected during surgery such as bowls, bladder, ureters, arteries etc. Finally, simplicity and accessibility can enable both low-volume and high-volume surgeons to rapidly remove the tissue once it has been placed into the container.

### SUMMARY

Some embodiments of a tissue containment and removal system may comprise a tissue container comprising: an opening; a multi-layer body that includes a combination of conductive material and non-conductive material forming an interior volume for receiving a tissue specimen through the opening; and a conductive connector coupled to the conductive material. The system may further include a cable extending between the conductive connector of the tissue container and a console for providing an electrical communication between the conductive material of the tissue container and the console, the cable having at least one elongate core having a structure that bears a tensile load for an electrode about the elongate core, the electrode forming a conductive path for the electrical communication with the conductive material of the multi-layer body.

Some embodiments of a tissue containment and removal system may comprise a tissue container comprising: an opening; a multi-layer body that includes both conductive material and non-conductive material forming an interior volume for receiving a tissue specimen through the opening; and a tissue cutter, wherein the tissue container is constructed and arranged to receive the tissue cutter through the opening of the multi-layer body about the tissue specimen, the tissue cutter having a tissue cutting blade that is configured to engage with a source of tissue inside the container, and wherein the bulk tissue reduce is automatically inactivated when the tissue cutter forms an electrical path with at least one strand of the conductive material of the multi-layer body of the tissue container.

Some embodiments of a tissue removal system may comprise a tissue container comprising: an opening; a multi-layer body that includes both conductive material and non-conductive material forming an interior volume for receiving a tissue specimen through the opening; a tissue cutter having a proximal end, a distal end, and a tube extending from the proximal end to the distal end, the tissue cutter including a tissue cutting blade at the distal end that is configured to engage with a tissue specimen inside the container; and a pressure or vacuum source at the proximal end for causing a change in pressure or force in an interior volume of the tube so that the tissue specimen is drawn toward the tissue cutting blade and into the interior volume of the tube.

Some embodiments of a tissue removal system may comprise a multi-layer bag with conductive and non-conductive elements on a deployable handle, comprising: an inflatable mechanism to unfold it after introduction into an abdomen which is electrically connected with a tissue reducing device to a console which can determine if the tissue reducing mechanism has contacted the multi-layer material of the container for the purposes of tissue removal in laparoscopic surgery.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 is an elevation view of a tissue containment and removal system embodiment.

FIG. 2 is a top view of a portion of a patient's body with a bulk tissue reducer embodiment and tissue container embodiment disposed within the patient's body cavity during a tissue containment and removal procedure.

FIG. 3 is an elevation view in section of the tissue containment and removal system embodiment of FIGS. 1 and 2 taken along lines 3-3 of FIG. 2.

FIG. 4 is a top view of the tissue containment and removal system embodiment of FIG. 1 with the distal end of the bulk tissue reducer disposed within an interior volume of the tissue container.

FIG. 5 is an elevation view in section of the tissue containment and removal system embodiment of FIG. 4 taken along lines 5-5 of FIG. 4.

FIG. 6 is a perspective view of a drive box embodiment.

FIG. 7 is a top view of the drive box embodiment of FIG. 6 with the upper cover removed for purposes of illustration.

FIG. 8 is a transverse cross section view of the tissue container of FIG. 1.

FIG. 9 is an enlarged view of the wall portion of the tissue container of FIG. 8 indicated by the encircled portion 9-9 in FIG. 8.

FIG. 10 is an enlarged view of the conductive layer of the tissue container wall of FIG. 9 taken along lines 10-10 of FIG. 9.

FIG. 11A illustrates a transverse cross section view of an embodiment of a multiple material cable.

FIG. 11B illustrates an elevation view partially cut away of the embodiment of the multiple material cable of FIG. 11A.

FIG. 11C is an enlarged view of a strand embodiment of a cable wire of the multiple material cable embodiment of FIGS. 11A and 11B.

FIG. 11D is an elevation view of a two-pin container plug at the distal end of the multiple material cable of FIGS. 11A-11C.

FIG. 12 is a schematic illustration of an embodiment of a counterfeit check system for a tissue container having a three-lead system.

FIG. 13 is a schematic illustration of a tissue container embodiment having a two lead system for a counterfeit check embodiment thereof.

FIG. 14 is a schematic representation in elevation of tissue container that includes an array of exposed electrical contacts for measurement of relative electrical resistance within an interior volume of the tissue container.

FIG. 14A is a schematic representation in a top view of the tissue container of FIG. 14 looking into the interior volume of the tissue container.

FIGS. 15A-15H illustrate embodiments of a variety of connector configurations for a tissue container cable that may be configured to prevent the use of counterfeit tissue containers in a tissue containment and removal system.

FIG. 16 is a schematic top view of an abdominal region of a patient including a plurality of incisions or ports for performing a laparoscopic procedure in accordance with embodiments of the present inventive concepts.

FIG. 17 shows an embodiment of a tissue container deployment device embodiment prior to deployment for use with tissue containment and removal system embodiments.

FIGS. 17A-17G illustrate features of a tissue containment and removal method sequence performed with the tissue container deployment device embodiment of FIG. 17 being used in a laparoscopic format.

FIG. 17H shows an embodiment of a tissue container deployment device of a tissue containment and removal system embodiment.

FIG. 17I shows a detailed view in section of the tissue container deployment device of FIG. 17H indicated by the encircle portion 17I in FIG.

FIG. 17J shows a detailed view in section of the tissue container deployment device of FIG. 17H indicated by the encircled portion 17I of FIG. 17H including an illustration of a combination of electrical, mechanical, and fluid connections of the tissue container deployment device embodiment.

FIGS. 18A-18G illustrate features of a tissue containment and removal method sequence being performed in a laparoscopic format.

FIG. 19 illustrates a schematic depiction of a tissue containment and removal system embodiment during use wherein the tissue specimen may be pulled through a tissue cutter utilizing a change in pressure or a force applied to the tissue specimen through a fluid.

FIG. 19A is an enlarged view of the distal end of the tissue cutter of the tisue containment and removal system embodiment of FIG. 19.

FIG. 20 illustrates a schematic depiction of a tissue containment and removal system embodiment wherein the tissue specimen may pulled through the tissue cutter with a change in pressure or a force applied through a fluid.

FIG. 21 is an enlarged view of a distal end of a tissue reducer embodiment of the tissue containment and removal system embodiment of FIG. 20 during use and illustrating a circulation path embodiment of the fluid.

FIG. 22 shows an embodiment of a tissue containment and removal system wherein the tissue container is in the form of a sheath that slides down the length of the tissue cutter during use.

FIG. 23 shows a distal end of the tissue container embodiment of FIG. 22 tied into a knot in order to seal the distal end of the interior volume of the tissue container.

FIGS. 24A-24D are schematic elevation views of different embodiments of tissue cutters that may be used with the various suitable tissue containment and removal system embodiments discussed herein.

FIG. 25 is an illustration of components of an embodiment of a tissue containment and removal system that may include an auto-shutoff feature as well as a tenaculum that may be used with the tissue containment and removal system.

FIG. 25A is a schematic depiction of the relative transverse dimensions of the bore diameter of a tissue cutter embodiment of a tissue containment and removal system embodiment and an outer transverse dimension of a tenaculum embodiment that may be used with the system.

FIG. 26 is a rear view of the bulk tissue reducer embodiment of FIG. 25 shown with an obturator embodiment disposed within the inner lumen of the tissue cutter of the bulk tissue reducer.

FIGS. 27A-27I illustrate an embodiment of a tissue containment and removal method sequence utilizing a tissue containment and removal system embodiment that includes a tissue container and a bulk tissue reducer.

FIG. 28A is a perspective view of a bulk tissue reducer embodiment.

FIG. 28B is an elevation view of the bulk tissue reducer embodiment of FIG. 28A.

FIG. 28C is a transverse cross section view of the bulk tissue reducer embodiment of FIG. 28B taken along lines 28C-28C of FIG. 28B.

FIG. 28D is an elevation view in section of the bulk tissue reducer of FIG. 28C taken along lines 28D-28D of FIG. 28C.

FIG. 29A is a rear view of a bulk tissue reducer embodiment with an obturator embodiment disposed within the inner lumen of the tissue cutter thereof.

FIG. 29B is an elevation view in section of the bulk tissue reducer embodiment of FIG. 29A taken along lines 29B-29B of FIG. 29A.

FIG. 29C is an enlarged view of the bulk tissue reducer of FIG. 29B indicated by the encircled portion 29C of FIG. 29B.

FIG. 29D is an enlarged view of the bulk tissue reducer of FIG. 29B indicated by the encircled portion 29D of FIG. 29B.

FIG. 29E is an enlarged view of the bulk tissue reducer of FIG. 29B indicated by the encircled portion 29E of FIG. 29B.

FIG. 30 is an exploded view of a tissue cutting blade assembly of the bulk tissue reducer embodiment of FIG. 29A.

FIGS. 31A-31C illustrate various views of housing components and the tissue cutting blade assembly of the bulk tissue reducer embodiment of FIG. 29A

FIG. 32 is an exploded view in elevation of the bulk tissue reducer embodiment of FIG. 29A.

FIGS. 32A-32C are transverse cross section views of various anti-rotation tube embodiments for use within the tissue cutting blade of bulk tissue reducer embodiments.

FIGS. 32D-32F are longitudinal section views of various anti-rotation tube embodiments for use within the tissue cutting blade of bulk tissue reducer embodiments.

FIG. 33 is an elevation view of an embodiment of a tissue container embodiment including a container conduit in electrical communication with a conductive layer of the tissue container.

FIG. 33A is an enlarged view of an electrical interface embodiment between the tissue container and container conduit of FIG. 33.

FIG. 34 illustrates an embodiment of a cable knot which secured a container conduit to a rim of a tissue container.

FIG. 35 illustrates an embodiment of a container conduit in electrical communication with the conductive layer of the tissue container and disposed in a looped configuration about a tissue container cable card embodiment.

FIG. 36 is an exploded view of a tissue container embodiment including an electrical interface between the tissue container and a container conduit.

FIG. 36A is an enlarged view of the tissue container embodiment of FIG.36 indicated by the encircled portion 36A-36A of FIG. 36.

FIG. 36B is an enlarged view of the tissue container embodiment of FIG.36A indicated by the encircled portion 36B-36B of FIG. 36A.

FIG. 36C shows an elevation view of a laminated mesh construction embodiment of a tissue container embodiment prior to being folding over, securing and sealing the edges of the laminated mesh.

FIG. 36D shows an elevation view of the tissue container of FIG. 36C after the edges of the laminated mesh construction have been secured and sealed together.

FIG. 36E shows an enlarged view in perspective of an electrical interface between the tissue container and the container conduit of the tissue container of FIG. 36.

FIG. 37 is a flow diagram illustrating an embodiment of a latch mechanism for providing an auto-shutoff function.

FIG. 38 shows a high level control schematic embodiment of a tissue containment and removal system embodiment.

FIG. 39 shows a high level hardware schematic embodiment of a tissue containment and removal system embodiment.

FIGS. 40A-40E show a cross section view of various embodiments of material composite configurations that may be used to form a wall portion of tissue container embodiments herein.

FIG. 41 shows an enlarged cross section view of an embodiment of a wall portion of a tissue container.

FIG. 42 shows an enlarged cross section view of an embodiment of a wall portion of a tissue container.

FIGS. 43A-43C show various views of an embodiment of a tissue container and subcomponents thereof that includes a first surface having a first color and a second surface having a second color different from the first color.

FIG. 44A is a perspective view partially cut away showing a tissue container embodiment in a deployed open state that includes an inflatable strip that may be used to facilitate unfurling of the tissue container.

FIG. 44B is a perspective view partially cut away showing a tissue container embodiment in a deployed open state that includes an inflatable strip that may be used to facilitate unfurling of the tissue container.

FIG. 44C is a perspective view partially cut away showing a tissue container embodiment in a deployed open state that includes an inflatable strip in the form of manifold having a plurality of longitudinally extending fingers that may be used to facilitate unfurling of the tissue container.

FIG. 44D is a perspective view partially cut away showing a tissue container embodiment in a deployed open state that includes an inflatable strip in a zig-zag or sinusoidal configuration that may be used to facilitate unfurling of the tissue container.

FIG. 44E is a perspective view showing a tissue container embodiment prior to deployment that includes an inflatable strip that may be used to facilitate unfurling of the tissue container.

FIG. 44F is a perspective view showing a tissue container embodiment prior to deployment that includes an inflatable strip that may be used to facilitate unfurling of the tissue container.

FIGS. 45 and 45A illustrate a schematic representation of a tissue containment and removal system embodiment including an ultrasonic morcellator embodiment that may be applied to a tissue specimen and used to morcellate the tissue specimen.

FIG. 46 shows a schematic representation of a tissue containment and removal system embodiment that may include a rotating blender-type morcellating blade.

The drawings are intended to illustrate certain exemplary embodiments and are not limiting. For clarity and ease of illustration, the drawings may not be made to scale, and in some instances, various aspects may be shown exaggerated or enlarged to facilitate an understanding of particular embodiments.

### DETAILED DESCRIPTION

As discussed above, devices and methods that provide for safe processing and removal of tissue specimens from a position within a patient's body, even in the possible presence of an occult malignancy, may be useful. Certain device and method embodiments for containing and removing tissue specimens from within a patient's body as well as related devices and methods are discussed in U.S. patent application Ser. No. 16/169,884, now U.S. Pat. No. 10,695,091 titled “Systems and Methods for Tissue Capture and Removal”, filed Oct. 24, 2018, by S. Kim et al., U.S. patent application Ser. No. 16/758,358, titled “Systems and Methods for Tissue Capture and Removal”, filed Apr. 22, 2020, by S. Kim et al., and International Application No. PCT/US2020/046135, filed Aug. 13, 2020, by J. Jones et al. titled “Tissue Removal Systems and Methods,” each of which is incorporated by reference herein in its entirety. Such devices and methods that function to safely remove tissue specimens from within a patient's body in a minimally invasive manner may be particularly useful.

Various applications may apply. In one application, such devices and methods are constructed and arranged for the containment, cutting, coring, and extraction of tissue during a laparoscopic hysterectomy. Here, a tissue containment and removal system are introduced trans-vaginally to isolate and contain benign tissue during powered bulk tissue reduction with a special-purpose morcellator or related tissue cutting apparatus. Any of the features, dimensions, or materials of the systems and methods of tissue capture and removal discussed in either of these incorporated references may be used in any suitable embodiment of the tissue containment and removal system embodiments or any associated devices or methods discussed herein.

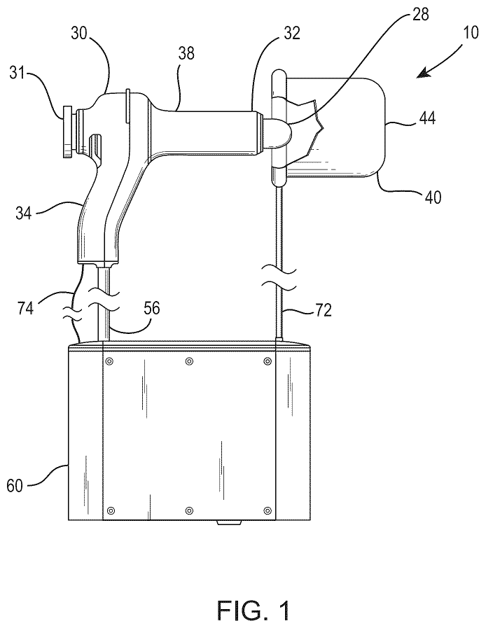

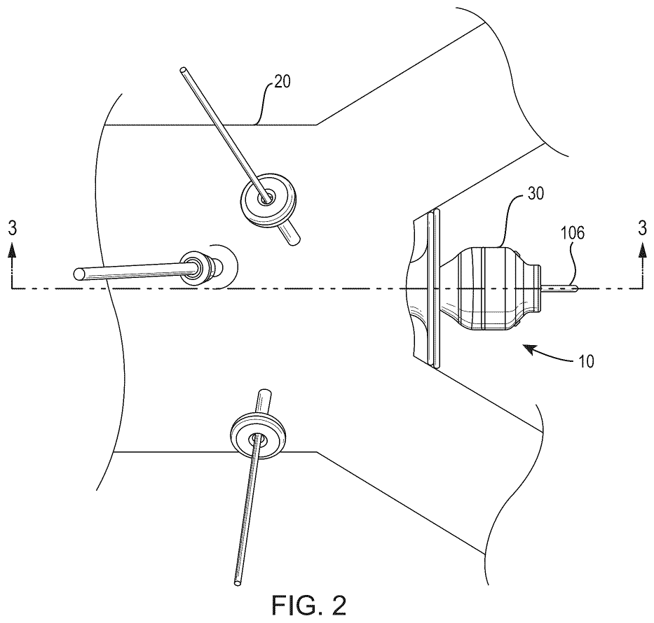

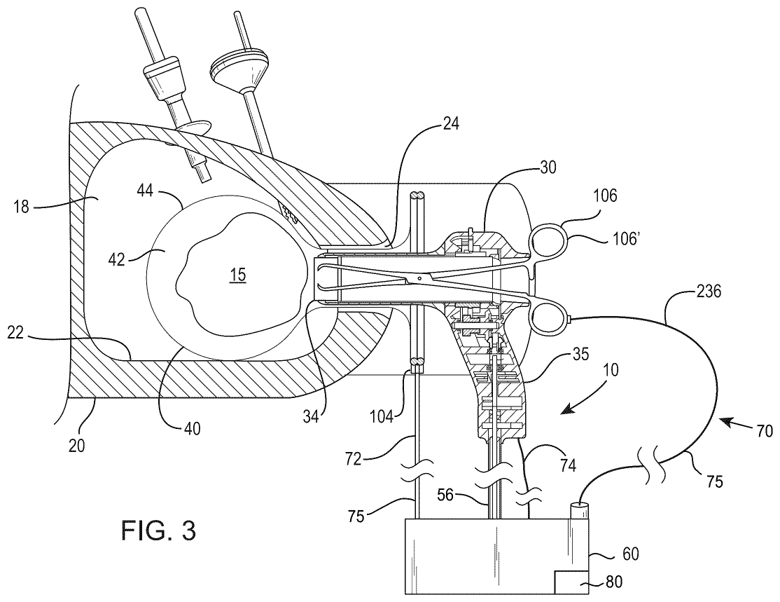

FIGS. 1-3 illustrate a tissue containment and removal system embodiment 10 that is configured to contain and isolate a tissue specimen 15 in situ within a body cavity 18 of the patient's body 20, and reduce or otherwise morcellate the tissue specimen 15 while the tissue specimen 15 remains disposed within the patient's body 20 and isolated from surrounding tissue 22 within the body cavity 18 as seen in FIGS. 2 and 3. In some cases, the tissue containment and removal system 10 may include devices and associated method embodiments for removing the morcellated tissue specimen 15 from the position within the patient's body 20 accessed through a body opening such as the vagina 24 as shown in FIG. 3. Other body openings 24 suitable for such access may include a surgically created incision in the patient's skin, fascia, abdomen, internal organs or the like or other natural body openings including the mouth, nostrils or anus. The tissue containment and removal system embodiment 10 shown may include a bulk tissue reducer embodiment 30 (which may also be referred to herein as a tissue morcellator embodiment) and a tissue container embodiment 40.

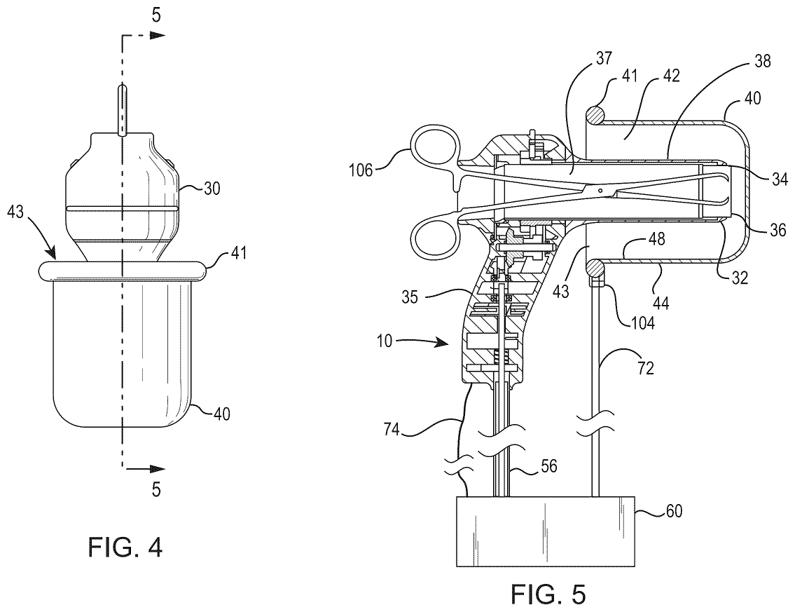

FIGS. 4 and 5 show the bulk tissue reducer 30 of the tissue containment and removal system 10 of FIG. 1 with a distal end 32 of the bulk tissue reducer 30 disposed within an interior volume 42 of a tissue container 40, also referred to as a bag. In order to facilitate introduction of the distal end 32 of the bulk tissue reducer 30 into the interior volume 42 tissue container 40, an obturator 31 may be disposed within an inner lumen or bore 37 of the tissue cutter 34 as shown in FIG. 1. In some cases, the obturator 31 may be generally cylindrical in shape with an outer transverse dimension that is a close sliding fit with the bore 37 of the tissue cutter 34, a length that is at least as great as a length of the tissue cutter 34 and a distal end 28 that has a rounded atraumatic bullet shape that is configured to extend beyond the distal end 33 of the tissue cutter 34 of the bulk tissue reducer 30 and ease introduction of the tissue cutter 34 and associated structures of the bulk tissue reducer 30 into the tissue container 40. Once the distal end 32 of the tissue cutter 34 is appropriately situated within the interior volume 42 of the tissue container 40, the obturator 31 used to guide the placement of the distal end 32 of the bulk tissue reducer 30 into the container 40 may be proximally withdrawn from the lumen or bore 37 and removed from the bulk tissue reducer 30.

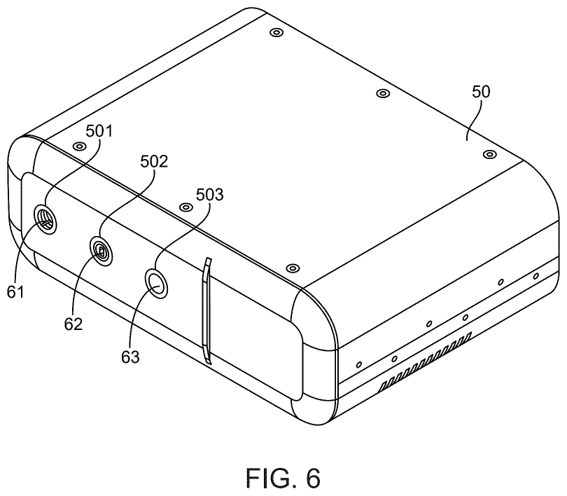

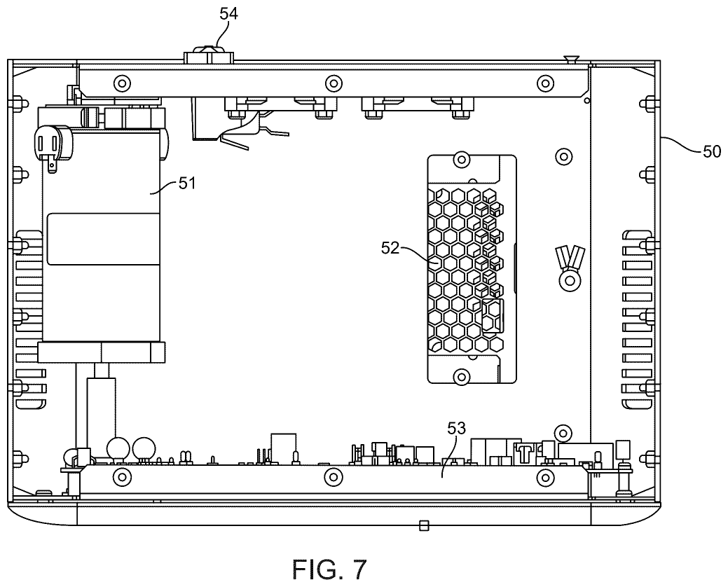

FIG. 6 depicts an embodiment of an optional drive box 50 that may be used for some tissue containment and removal system embodiments 10 to provide rotational energy to a tissue cutter 34 (see FIGS. 3 and 5) of the bulk tissue reducer embodiment 30. FIG. 7 depicts the drive box embodiment 50 without the top cover and illustrates internal component embodiments of the drive box embodiment 50. For some embodiments, the drive box 50 may include a motor 51, a power supply 52, a circuit board 53, and a connector 54 that may be configured for operative coupling to the bulk tissue reducer 30. These components of the optional drive box embodiment 50 may be included in the console embodiment 60 of the tissue containment and removal system embodiment 10 shown in FIG. 1. In some embodiments, the motor and battery can be part of the bulk tissue reducer embodiment 30, for example, positioned in the handle of the cutter, in which case the drive box 50 is not required.

As shown in FIG. 6, the front panel includes various respective connectors and indicator lights. In order for a tissue cutter of the bulk tissue reducer to operate, embodiments of the system must detect that a viable container 40 is attached to the drive box 50 included in the console 60, as distinguished from a counterfeit container or any other unsuitable container. The drive box 50 is reusable and constructed and arranged to provide power to the bulk tissue reducer 30. In doing so, the drive box 50 includes a first connector 61 for receiving and removably coupling a drive cable 56 of the bulk tissue reducer 30 and a second connector 62 for receiving and removably coupling a container cable 72 (also referred to as a tether, conduit, or electrode and described below). The indicators 501, 502 and 503 disposed about the connectors 61-62 as well as the reset button 63 may be LEDs or other light illumination devices. When the drive cable is properly inserted into the first connector 61, the first indicator 501 may illuminate a color, e.g., green, or change colors, for example, from orange to green. Similarly, when the container cable 72 is properly inserted into the second connector 62, the second indicator 502 may illuminate a color, e.g., green, or change colors, from orange to green. When both connectors 61, 62 are inserted into the drive box 50 and the corresponding indicators 501, 502 indicate that the connections are proper, i.e., permitting power and/or data signals to be exchanged between the drive box 50 and container 40 and bulk tissue reducer 30, respectively, the system can change states, from a standby mode to a ready mode, for example, by pressing a standby/ready mode button 63 on the console 50, whereby the third indicator 503 can illuminate a color, e.g., or change colors, for example, from orange to green. A determination of whether a container is counterfeit can be established electrically by the console 60 (based on various impedance and resistance checks in some cases) and if the container 40 is deemed viable then the container indicator 502 for the container turns green.

If both the handpiece indicator 501 and container indicator 502 are green, then the system can transition from standby mode into ready mode. When all three indicators 501-503 are green, the system is ready to run. If at any point, the console 60 electrically detects that either the container 40 or the handpiece is disconnected or damaged, the system may be configured to immediately stop running and enter standby mode by executing an embodiment of an auto-shutoff feature (described below). As described below with respect to embodiments of an anti-shutoff feature, the user then can choose a variety of troubleshooting options including: 1) Inspection and insertion/reinsertion of the container or handpiece to console connections 2) Replacement of the handpiece or container or console components or 3) Shift out of standby mode and potentially continue the surgery.

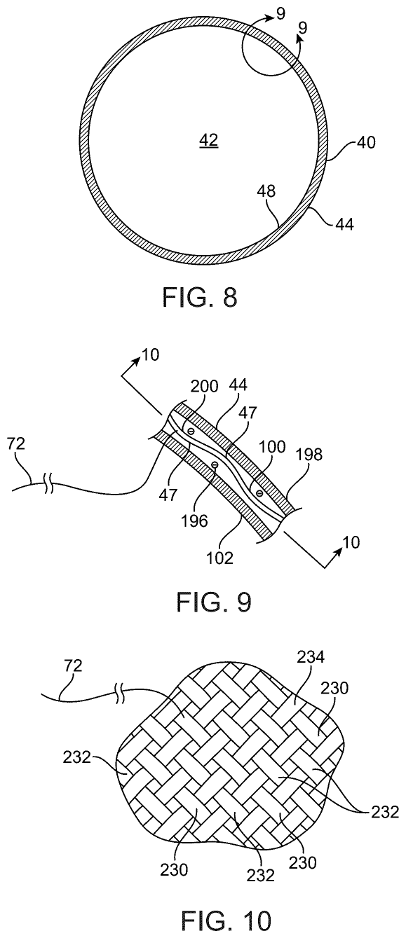

For some tissue containment and removal system embodiments 10, the tissue cutter 34 of the bulk tissue reducer 30 may be operated within the interior volume 42 of the tissue container 40. Because some tissue container embodiments 40 may have a thin flexible wall structure 44, it may be important to prevent contact between the wall 44 of the tissue container 40 and a tissue cutting blade 36 of the tissue cutter 34 that might result in damage to or puncturing of the wall 44 of the tissue container 40. As such, some tissue containment and removal system embodiments 10 may include a contact detection system, for example, described in International Application No. PCT/US2020/046135, filed Aug. 13, 2020, by J. Jones et al. titled “Tissue Removal Systems and Methods,” which is incorporated by reference above, that may be configured to emit a warning signal and that may optionally include an auto-shutoff feature for use when contact or near contact is made with the wall 44 of the tissue container 40 or certain components, such as a conductive element 47 as shown in FIGS. 8 and 9, thereof by the tissue cutting blade 36.

In order to contain and isolate a tissue specimen 15 prior to reduction or morcellation of the tissue specimen 15, it may be desirable to reliably and consistently deploy a suitable tissue container embodiment 40 around the tissue specimen 15 in some cases. This process may typically be carried out in the confined space of the body cavity 18 within the patient's body 20. In some cases, this process may be facilitated by the use of a suitable container deployer or container deployer assembly described in International Application No. PCT/US2020/046135, filed Aug. 13, 2020, by J. Jones et al. titled “Tissue Removal Systems and Methods,” which is incorporated by reference above.

With further regard to FIGS. 6 and 7, certain system embodiments, subassemblies or components thereof discussed herein may benefit from particular characteristics for various different indications, operating conditions etc. As such, any of the system embodiments, subassemblies or components thereof discussed herein may include any of the following features, dimensions or materials or suitable combinations thereof. In particular, the torque of the motor 51 may range in some cases from about 1 inch-ounce to about 50 inch-ounces. Or more specifically about 1 in-oz to about 28 in-oz. In some instances, torque requirements at the cutting edge of the tissue cutter may range from no-load conditions to threshold conditions. For some indications, such as a transvaginal application, a tissue cutter with diameter of about 20 mm to about 50 mm may require as much as about 10 in-oz. to about 20 in-oz of torque when cutting. A tissue cutter used in a transabdominal application wherein the tissue cutter diameter may be between about 3 mm and 25 mm may only require between about 1 in-oz. and about 18 in-oz of torque when cutting. In addition, gear ratios and rotations per minute (RPM) may impact the force applied to the cutting edge of the various cylindrical cutter embodiments. For some embodiments, the outer diameter of the cannula 38, may be up to about 42 mm in some instances while the inner diameter of a corresponding tissue cutter, such as tissue cutter 34, may be up to about 34 mm. In some cases, the outer transverse dimension or diameter of the tissue cutter 34 may range from about 2 mm to about 60 mm with a corresponding inner transverse dimension or diameter of about 1.5 mm to about 55 mm. For some embodiments, an inner transverse dimension or diameter of the opening 43 of the container 40 may be about 10 mm to about 450 mm, more specifically, about 150 mm to about 180 mm, in some cases.

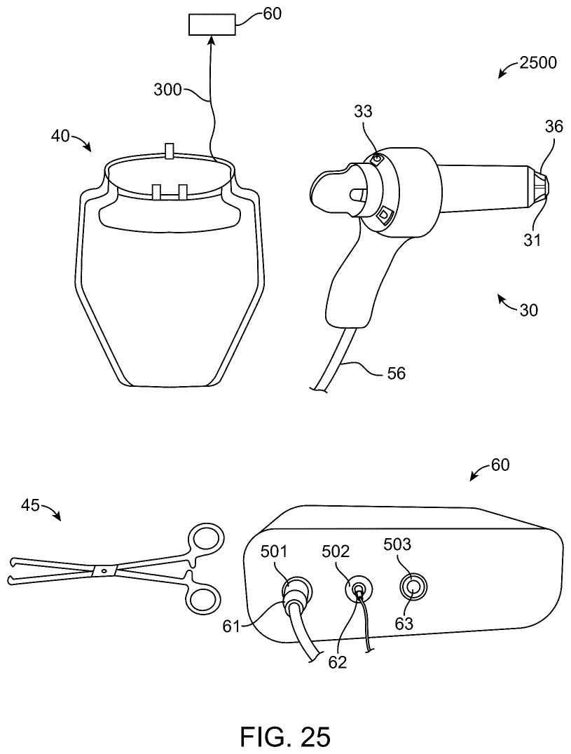

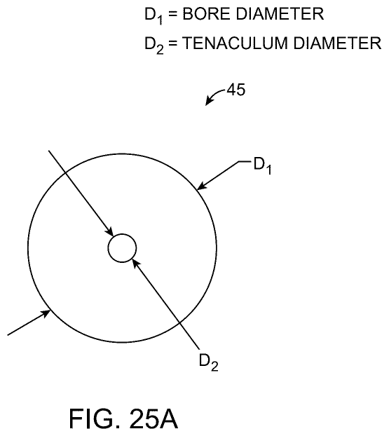

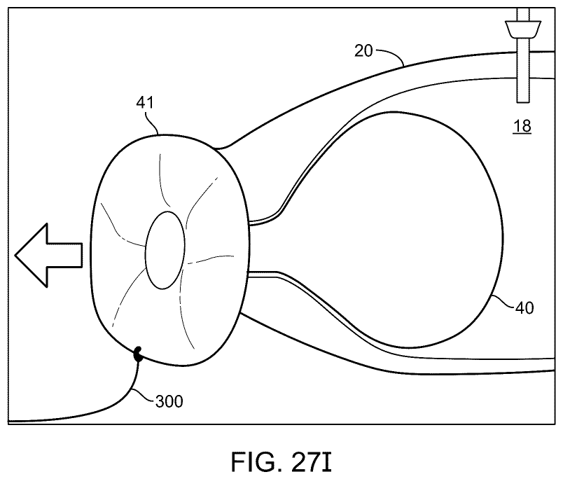

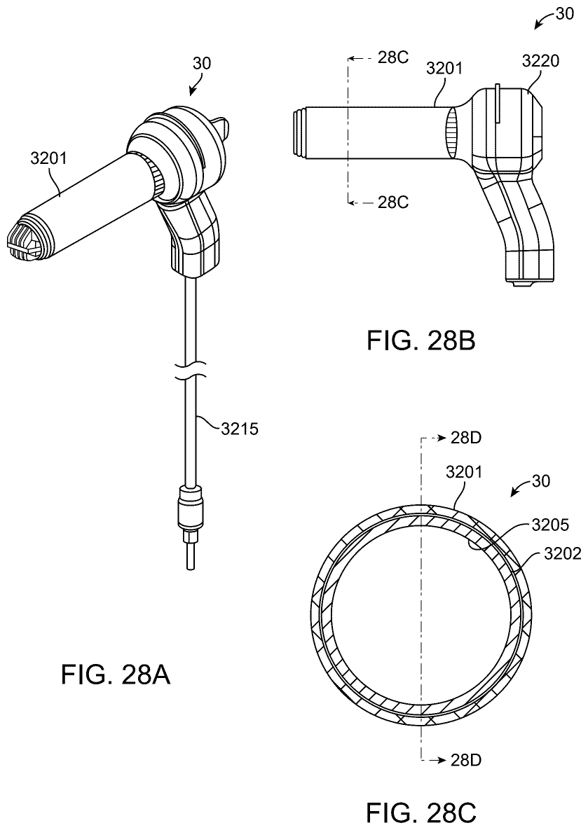

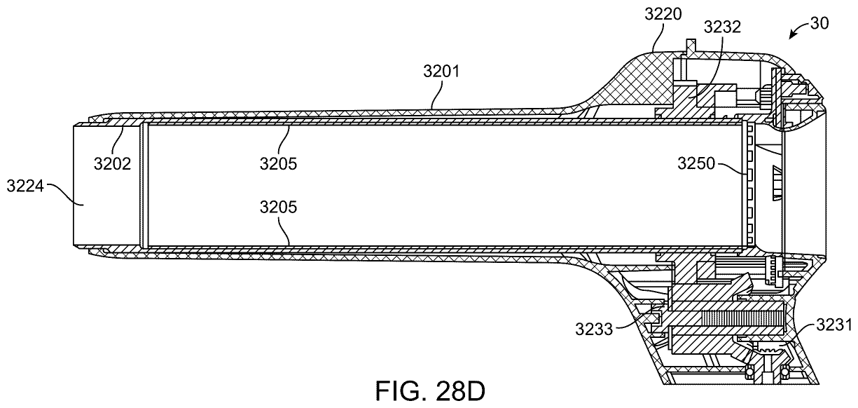

FIG. 3 shows the tissue containment and removal system embodiment 10 of FIG. 1 that includes an embodiment of the contact detection system 70 with an auto-shutoff feature, described in detail with reference to FIGS. 25, 37, 38, and 39. The tissue containment and removal system 10 having the auto-shutoff system is shown with the distal end 32 of the bulk tissue reducer 30 thereof disposed within the interior volume 42 of the tissue container 40 and adjacent a tissue specimen 15 within a pelvic cavity 18 of the patient 20. For such a tissue containment and removal system embodiment 10, by attaching the container cable 72 in electrical communication with the conductive element 47 of the tissue container 40 and a blade conduit 74 (second electrode) to a conductive portion of the tissue cutting blade 36 of the bulk tissue reducer 30, an electrical circuit may be closed when there is physical and electrical contact between the tissue cutting blade 36 and the conductive element 47 of the tissue container 40 and electrical current travels from one to the other. In some cases, this completion of the circuit may be used by a controller 80 to identify the generally undesirable condition that occurs when the tissue cutting blade 36 of the bulk tissue reducer 30 has come into contact or close proximity with the wall 44 of the tissue container 40. The motor of the bulk tissue reducer 30 may be disposed either in the body of the tissue cutter or in the drive box 50 where it may be operatively coupled to the tissue cutting blade of the bulk tissue reducer 30 by a flexible shaft 56 which is configured to transmit rotational torque from the motor (or a suitable gear system coupled thereto as shown in FIG. 28D) to the tissue cutter 34 of the bulk tissue reducer 30.

For some tissue container embodiments 40, the conductive layer 47 may include a woven mesh 100 as seen in FIG. 9 with a composite mesh structure that includes both strands of conductive material and strands of non-conductive material as shown in FIG. 9. In general, conductive layer embodiments 47 and non-conductive layer embodiments (discussed below) of tissue container embodiments 40 discussed herein may include materials such as any suitable biocompatible material, including plastics such as polyethylene, polyurethane, polypropylene, PET, PETG, aramid and para-aramids, including, e.g., poly-paraphenylene terepthalamide (Kevlar®), aliphatic or semi-aromatic polyamides (NYLON®), Spectra® fibers, rubber, thermoplastics and others. Other embodiments of multilayer materials forming a tissue container are described below. For example, the container body may be formed of a multilayer material, including a conductive layer that is electrically coupled via an auto shut-off system embodiment to a cutting instrument, may be used for materials such as material sheets or layers that can be used to drape over (and therefore protect) a patient during surgery. Such multilayer materials can be used as a protective liner during surgery and can coat any object that needs to be protected in a surgical environment from surgical cutting instruments. This protection may include protection from blunt trauma of the instrument and also from fluid ingress.

Referring to FIGS. 8 and 9, for some tissue containment and removal system embodiments 10, the tissue container 40 may include a second non-conductive layer 102 which is disposed on an inside surface 196 of the conductive layer 47. The tissue container 40 may also additionally include a third non-conductive layer 198 disposed on an outside surface 200 of the conductive layer 47. The conductive layer 47 disposed between the second layer 102 and the third layer 198 may include a composite weave having non-conductive strands interwoven with conductive strands which make up the conductive element 46 of the tissue container 40. For some other embodiments, the conductive layer 47 and conductive element 46 thereof disposed between the second layer 102 and the third layer 198 may include a wire mesh 100 made some or entirely from conductive strands 230. The conductive strands 230 of the wire mesh 100 may include or be made from metals like stainless steel in some instances.

FIG. 10 shows how the middle layer 47 of a layered wall 44 of the tissue container 40 may be made of a composite weave 234 for some embodiments. This composite weave 234 may include or otherwise be made of strands 230, 232 that include materials such as polyester, polyethylene, metal, stainless steel, or any combination of metal and plastic or polymer. For some composite weave embodiments 234, the ratio of polymer strands 232 to metal strands 230 in the composite weave 234 may, in some cases, range from about 20% to about 80%, or from about 10% to about 90% in other cases or from about 1% to about 99% in still other cases.

Some tissue container embodiments 40 may include the interior volume 42, the opening 43 and the conductive layer 47 which includes the composite weave 234 having conductive strands 230 and non-conductive strands 232. For some of these tissue container embodiments 40 the non-conductive strands 232 may include a polymer such as polyester, polyethylene, Kevlar®, Spectra®, or nylon. For some of these tissue container embodiments 40 the conductive strands 230 may include a metal such as stainless steel, nickel titanium alloy or the like. The conductive strands 230 and non-conductive strands 232 may, in some cases, have an outer transverse dimension of about 0.01 mm to about 0.5 mm. For tissue container embodiments 40 having a composite weave 234, a variety of ratios for non-conductive strands 232 to conductive strands 230 may be used. For some embodiments, the ratio of non-conductive strands 232 to conductive strands 230 may be about 10% to about 90%, more specifically, about 20% to about 80% as well as any other suitable ratios as discussed herein. Referring to FIGS. 8-10, for some embodiments, the tissue container 40 may optionally further include a second non-conductive layer 102 disposed on an inside surface 196 of the conductive layer 47, a third non-conductive layer 198 disposed on an outside surface 200 of the conductive layer 47 or both of these additional layers.

Referring again to FIG. 3, an optional conductive tenaculum conduit 236 is shown that serves to electrically couple the tenaculum 106 to the contact detection system 70 and detection circuit 75 thereof in a manner similar to the coupling of the tissue cutting blade 36 of the bulk tissue reducer 30 to the contact detection system 70. As such, FIG. 3 also shows a contact detection system embodiment 70 that is operatively coupled between a surgical instrument in the form of the tenaculum embodiment 106 and the conductive element 46 of the tissue container embodiment 40. Such contact detection system embodiments 70 may thus detect when the tenaculum 106, or any other suitable coupled surgical instrument, breaks through the non-conducive layer 102 and comes into contact or close proximity with the conducive element 46 of the tissue container 40. This configuration may be used to alert the user that the tissue container 40 has been breached, compromised or is about to be contacted by the tenaculum 106 or any other suitably configured instrument that may be useful when disposed within the interior volume 42 of the tissue container 40 during a tissue removal procedure.

The tissue container 40 may also additionally include the third non-conductive layer 198 disposed on the outside surface 200 of the conductive layer 47. The conductive layer 47 disposed between the second and third layers 102, 198 may include the composite weave 234 having non-conductive strands 232 interwoven with conductive strands 230 which comprise the conductive element 46 for such a tissue container embodiment 40.

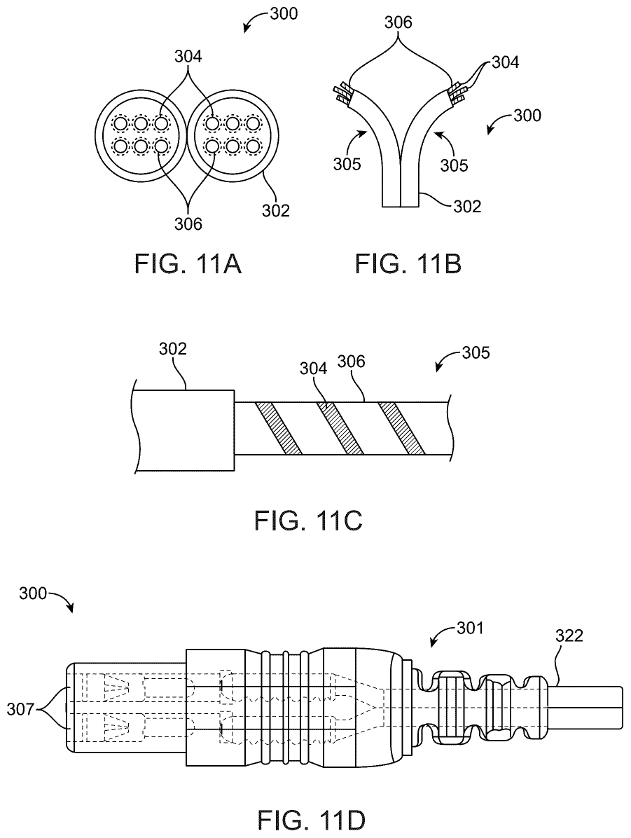

FIGS. 11A-11D are schematic views of an embodiment of a multiple material cable 300 configured for extending between a console 60 and a container 40 of a tissue containment and removal system. In some embodiments, the multiple material cable 300 may be the same as or similar to the container cable 72 described above and may therefore also be referred to as a tether, conduit, or electrode. While performing minimally invasive procedures that include the use of a tissue containment and removal system 10 above, a user may apply high forces on the conduit 300, which can damage the conduit 300 or its connections to the console 60 and/or container 40. As described herein, the multiple material cable 300 is constructed and arranged to accommodate such high tensile loads without damage or reduced performance of these components and to provide strain relief to the conduit 300.

The cable 300 may be formed of one or more strands 304, also referred to as durable tensile cores that are configured to carry a majority of the tensile load imposed on the cable 300, and at least one electrode element 306 which may be wrapped around each strand 304 or otherwise extend along a longitudinal length of the cable 300. The cores 304 may be disposed in any suitable configuration that accepts the majority or core of the tensile load that migh otherwise be imparted to the electrode element 306. A plurality of strands, for example, 1-20 tensile cores, may be used to form a cable wire 305. In a particular embodiment, a single cable wire 305 may be formed of four strands 304 surrounded by an insulative sheath 302. A plurality of wires 305 may be combined into the same cable 300. The cable 300 may be specifically rated for high tensile loads so that its mechanical and electrical integrity is maintained during manipulation while in use, for example, when forces are applied that stretch, bend, or otherwise contort the cable 300. The configuration of the multiple material cable 300 may be such that any initial tensile load borne by the electrode element 306 is transferred onto the core 304 after the cable 300 has been placed under tension.

The strand cores 304 may typically include materials such as aramid such as Kevlar® or Nomex®) or polyester to provide mechanical rigidity. The electrode element 306 wrapped around the core 304 may be arranged as one or more flattened strips or the like made of standard conductive materials commonly used in electrodes such as copper or aluminum. These flattened metal strips 306 which can also be referred to as tinsel and may be plated with other highly conductive metals, such as silver. The tinsel 306 may also include a single core wire or includes multiple smaller conductive strands. These bundles of strands may then be sheathed in a plastic extrusion such as polyvinylchloride (PVC) or polyurethane for forming the insulative sheath 302. Under tension, the cores 304 bear the majority of the tensile force because they are in a straight orientation. In other words, a core 304 due to its mechanical rigidity can endure the tensile force and resist deformation or strain in a linear direction and the tinsel electrode element 306 is wound helically around the core 304 so an applied tensile load tightens around the core 304 so that the tensile load is borne by the core 304 instead of the electrode element 306, which can reduce the risk of severing or otherwise damaging the electrically conductive path extending from the console 60 to the container 40 provided by the electrode element 306. In some embodiments, such a tinsel-configured electrode element 306 may be disposed in a helical orientation, so a tensile load will cause it to tighten around the core 304, rather than resist in the direction of the tensile force. Thus, the tensile load is borne by the high-strength core 304 (for example, the ultimate tensile strength of Kevlar® is closer to 3000 MPa) instead of the conductive tinsel (for example, the ultimate tensile strength of copper is around 210 MPa). In embodiments where the electrode element 306 is formed of a plurality of conductive strands, strands of the electrode element 306 also can be twisted. Any helical arrangements may equally apply such that initial high loads might untwist them first, allowing more elasticity with the electrode and less with the sheath 302, thus transferring the tensile load onto the sheath 302 from the core electrical lead. As shown in FIG. 11D, one application of the cable 300 is coupling one end to a plug 307 or other connector, for example, a two-pin connector (each pin corresponding to a cable wire 305) for insertion into the connector 62 of the drive box 50 of the console 60 shown in FIG. 6.

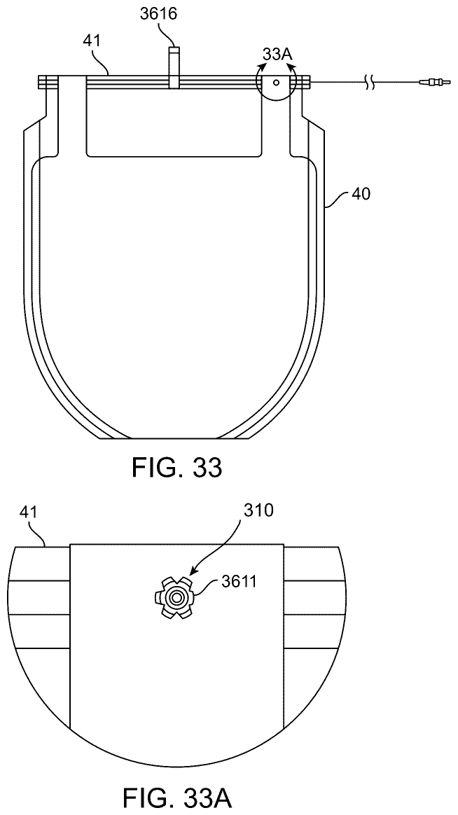

As described below, the other end of the cable 300 is configured for electrically coupling to the container 40. Another cable embodiment (not shown) could have a conductive and tensile element simply adjacent to one another, or separate from one another, not helically intertwined. Along these lines, referring again to FIG. 3, In some embodiments, the snap connector 104 between the conductive rim 41 of the container 40 and the cable 72, 300 may not be required to form electrical continuity between the cable 72, 300 and electrode or tether/electrode combination, but rather a rigid electrical connection may be made directly from the tether/electrode combination directly to the conductive layer 47 in the tissue container 40 to establish the electrical continuity therebetween. Instead of a snap feature or the like, this connection can be made from a mechanical connection such as a metal star-shaped spike washer, crimp, rivet, or grommet, or any penetrating washer for example, shown and described in FIGS. 33-36. In other embodiments, these mechanical connections may be reinforced in conjunction with solder and/or glue.

As shown in FIGS. 33-36, one example of such a mechanical connection 310 may include a combination of a washer 3612, rivet 3611, and ring terminal crimp 3613. Here, the spike washer 3612 may be used to pierce and make electrical contact with the metal layer of the of the multilayer container 40. It may then be fastened into place by the rivet 3611. The spikes or sharp edges of the washer 3612 may then be bent down to secure the ring terminal 3613, which is crimped to the electrically conductive cable 300. A washer 3611 such as the spike washer may be used as a reliable multi-point method of penetrating the container laminate material, e.g., shown in FIGS. 8-10, to provide a secure, low resistance electrical connection to the stainless-steel conductive layer.

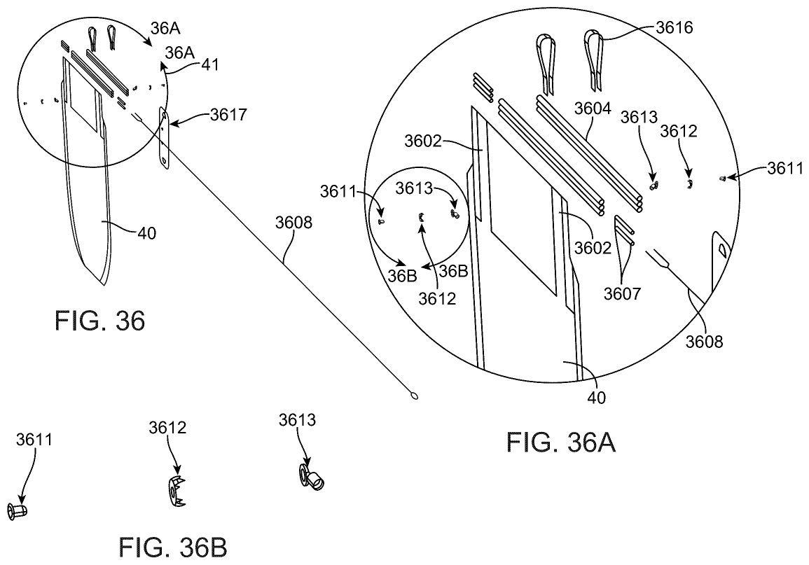

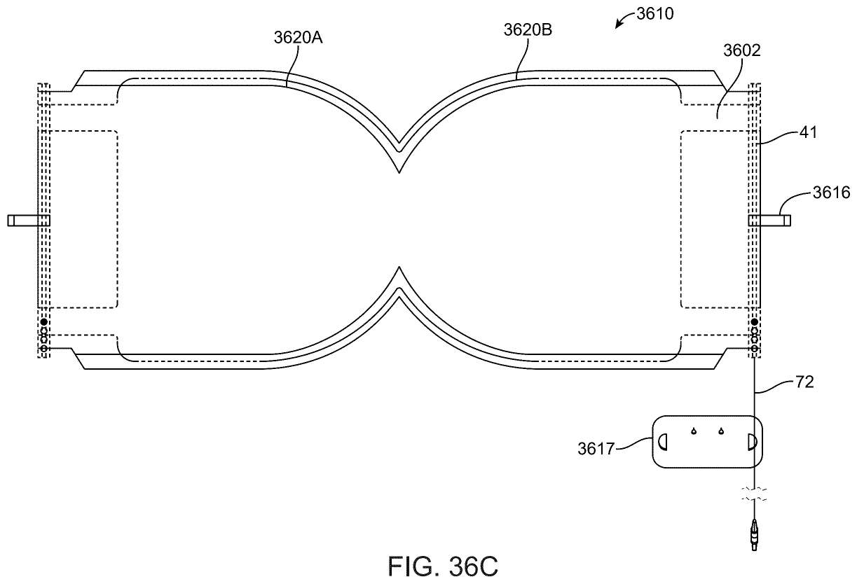

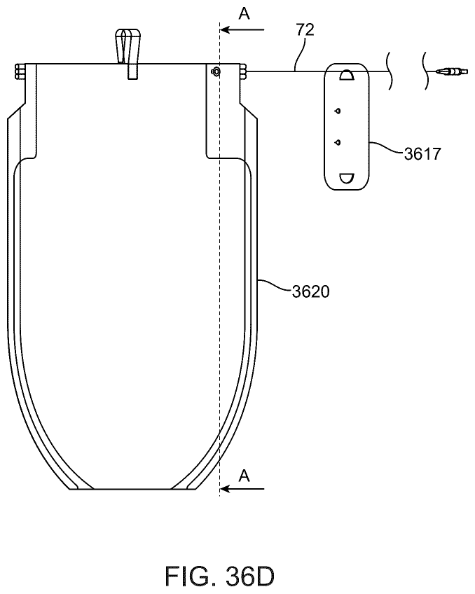

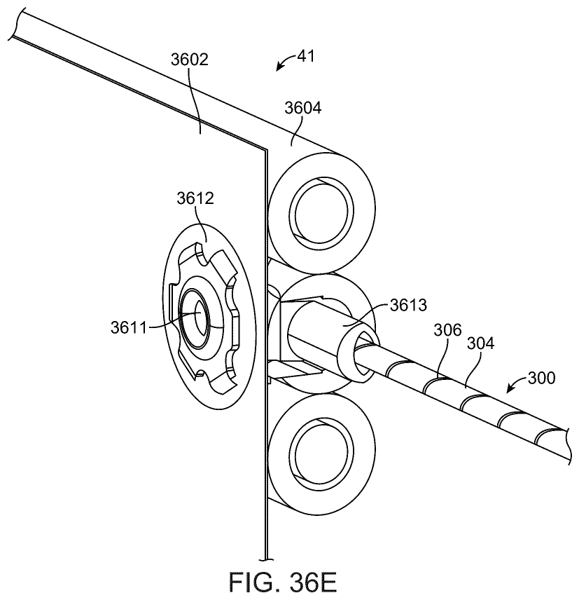

In some embodiments, as shown in FIG. 36, two elongated portions 3602 of the container 40 may extend from a main body of the container 40. The elongated portions 3602 may be formed in a same manner as a main body, for example, a laminated mesh 3610 shown in FIG. 36A, or other embodiments of the container 40, for example, described herein. The rim 41 can be coupled to the elongated portions 3602, for example, described in FIGS. 33 and 33A, for example, using the rivet or eyelet 3611 or grommet or the like, washer 3612 such as a spike washer, and ring terminal 3613 to provide a conductive connection between the rim 41 and conductive elements extending through the elongated portions 3602 and the container body. The rim 41 may further include one or more tubes, tube ribbons, or other elongated elements 3604 formed of polyurethane or other polymer and/or natural material. In some embodiments, the foregoing can be at an edge of the container 40 in the absence of a physical rim. The tubes 3604 may be coupled to the elongated container portions 3602 by the combination of eyelet 3611, spike washer 3612, and ring terminal 3613, and/or other coupling mechanisms. Other components may include a PTFE beading 3607, and conductive element 3608 such as a cable, tinsel wire, etc. which is configured to be in direct contact with container cable card 3617. In some embodiments, the conductive element 3608 may be the cable 300 described in FIGS. 11-11C. With further reference to FIG. 36C, two halves of the container body are shown which when folded and sealed at the laminated edges 3620A, B (generally, 3620) can form the container, for example, shown in FIG. 36D, having an interior volume.

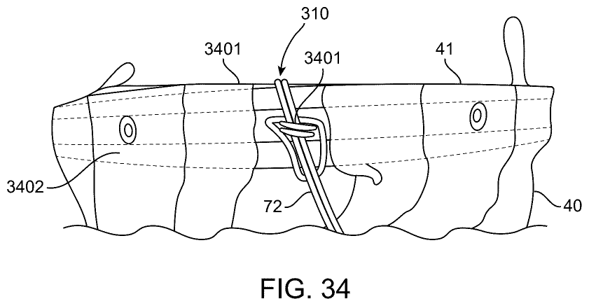

As shown in FIG. 34, cable knot embodiments may be used to help provide a secure method of acting as a strain relief so that the cable 72 does not move much with respect to an electrical connection. This may limit the mechanical load on the electrical connection to prevent the wires or any other type of conductor from pulling out of the ring terminal crimp. There are many types of suitable knot embodiments that may be used. In some embodiments, an adhesive such as a Loctite light cure acrylic adhesive, may be applied on the knot 3401, and/or electrical connections 3402 between the cable 72 and container rim 41. The electrical connection 3402 may be formed by a spike washer, rivet, and/or other coupling technique described herein.

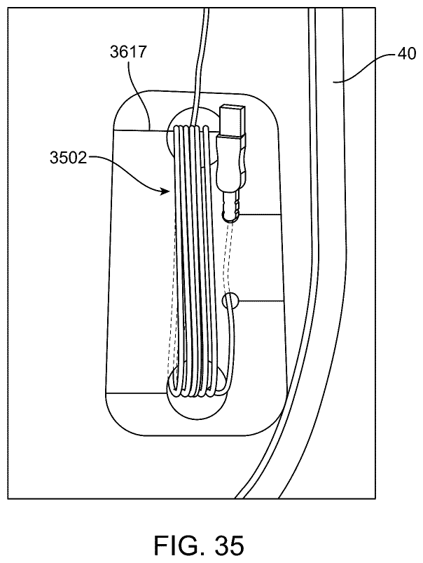

In other embodiments, as shown in FIG. 35, the cable may be looped onto a card to help manage the cable 3502. Cable card embodiments 3617 may be used to help manage the electrical cable/mechanical tether such that it can be handled appropriately in the sterile field. Embodiments of the cable card may include those that are connected to the container itself, configurations that are designed to be placed onto the patient, or a configuration that is designed to be placed on the operating room table as the container is loaded into the vaginal canal. The embodiment shown in FIG. 35 is free floating and the card is approximately 6-9 inches from the edge of the tissue container 40.

The tissue container 40, or more specifically the rim 41 of the container 40, may have a connector comprising one or more conductive leads, e.g., up to four leads in some instances, but not limited thereto. The single, dual, and multiple lead connectors may be used for a variety of purposes including detecting if the tissue container 40 has any damage, whether a counterfeit container has been connected to the console 60, to determine if any of the connections or connections from the electrode wire to the conductive layer of the container 40, e.g., at the rim 41, have been compromised, to determine the quantity of conductive fluid within the container 40, and to detect the quantity of tissue disposed within the interior volume of the container 40.

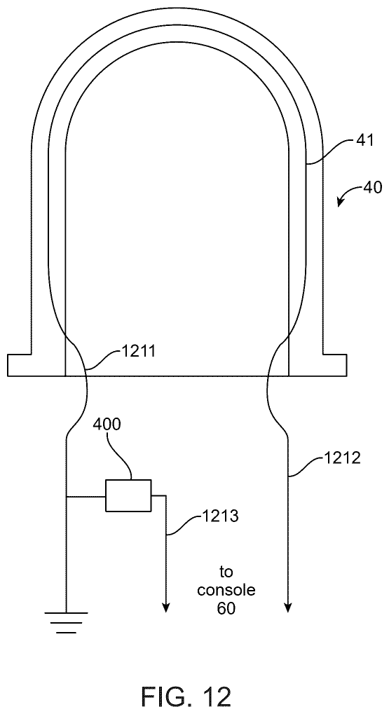

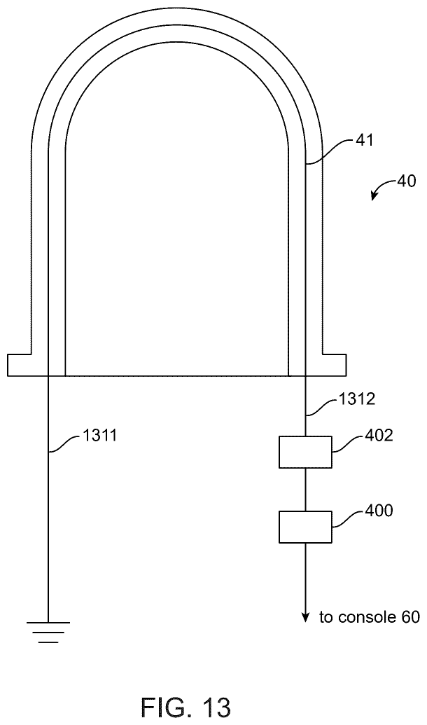

A three-lead system may include a counterfeit check system 400 as shown in the FIG. 12 to authenticate the container 40. Here, two of the leads 1211, 1212 (referred to as wires or conductors) extend from opposite sides of the container 40 to a source and ground, respectively to ensure that there is a sufficient electrical connection to the container 40 from the source, e.g., from the console 60. In some embodiments, the leads 1211, 1212 may be part of a cable 72, 300 described herein. For example, as shown in FIG. 11D, the leads 1211, 1212 can form the two-pin connector 307 terminating at the plug 301. A counterfeit check system 400, which may be implemented as an integrated circuit in a computer chip or the like, may be coupled between the source wire 1211 and a wire 1213 connected to the console 60, for example, where wire 1213 extends to a microprocessor of the drive box 50 included in the console 60 shown in FIG. 7. In some embodiments, the counterfeit check chip 400 is embedded in a container connector. The wire 1213 to the counterfeit check chip 400 (on the non-ground side) connected to the console microprocessor can exchange signals over the wire 1213 that confirm whether a valid and unused container 40 is attached, for example, and write to the chip indicating that it has been used. In other embodiments, as shown in FIG. 13, two wires 1311 and 1312 may be used. Here, the counterfeit check system 400 and an attachment detection system may be implemented as chips or the like and extend serially along the non-ground wire 1312 of the cable 300. In some embodiments, the connector to the console 60 may have three contacts: two container conductors and a conductor for the counterfeit detection system 400 and/or attachment detection system 402.

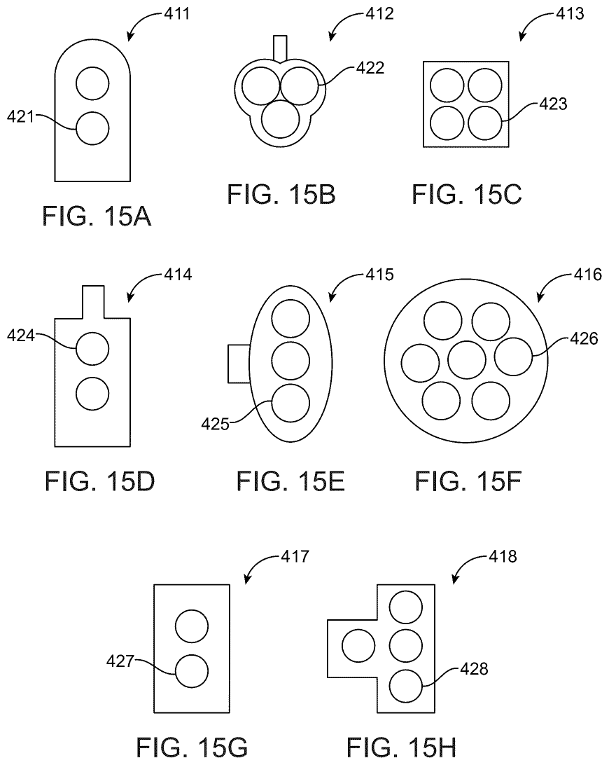

The counterfeit check system 400 could include an element having a known resistance correlating to a lot number or it could be a more sophisticated microprocessor that was encrypted with specific bit numbers correlating to either a handpiece or a lot number. Additionally, the shape of the cross-section of the connector, for example, shown in FIGS. 15A-15H of the tissue container 40 may be unique to prevent counterfeit containers from being connected to the console 60. In particular, each container connector 411-418 may have various arrangements of contact points 421-428. For example, the contact points 421-428 can be formed according to a tinsel/core arrangement shown in FIGS. 8-11.

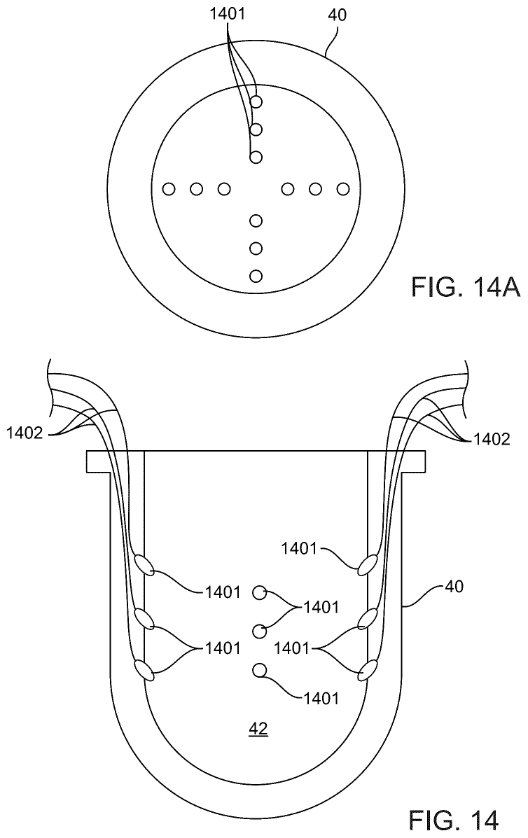

Additionally, if the wires 1402 embedded in the tissue container 40 are partially exposed to form an array or pattern of electrical contact points 1401 throughout the interior of the tissue container 40, as shown in FIG. 14 or an electrically conductive layer of the multilayer tissue container 40 is so exposed in certain locations, the change in resistance measured between such electrical contact points 1401 which are disposed in electrical communication with the interior volume 42 of the tissue container 40 can provide an indication of whether the interior volume 42 of the container 40 is empty in that specific location or not. It may also be used to determine if the interior volume 42 of the tissue container 40 was filled with tissue or fluid For example, if there is a high resistance, that portion of the tissue container 40 is empty and that particular electrode or node 1401 is in contact with air, if it is a low resistance, that portion of the tissue container 40 is full or in contact with fluid or tissue. This could be useful for providing feedback to the surgeons in a blind procedure to determine how much tissue is remaining in the tissue container 40. If, for example the tissue container 40 is empty, then the surgeon or user would know to remove the bulk tissue reducer 30 and not try to grasp tissue 15 anymore with the tenaculum 106 or suction, and to attempt to pull the tissue container 40 out of the patient's body cavity 18.

Conversely, if the tissue container 40 does have tissue remaining in it, the surgeon should continue to keep the bulk tissue reducer 30 or tissue reducer into the interior volume 18 of the tissue container 40 and continue extracting tissue 15 rather than attempting to pull the tissue container 40 out of a hole, incision, or orifice 24 which the tissue container 40 will not fit out of due to the quantity of tissue 15 remaining in the tissue container 40. The user interface of this system may function either as a visual display of which nodes 1401 are in contact with tissue 15, alternatively the user experience could be such that a tone, light, alert, or other display alert is provided to the user once the tissue container 40 is empty (as determined that all contact points, or electrode points 1401, are in contact with air or air equivalent).

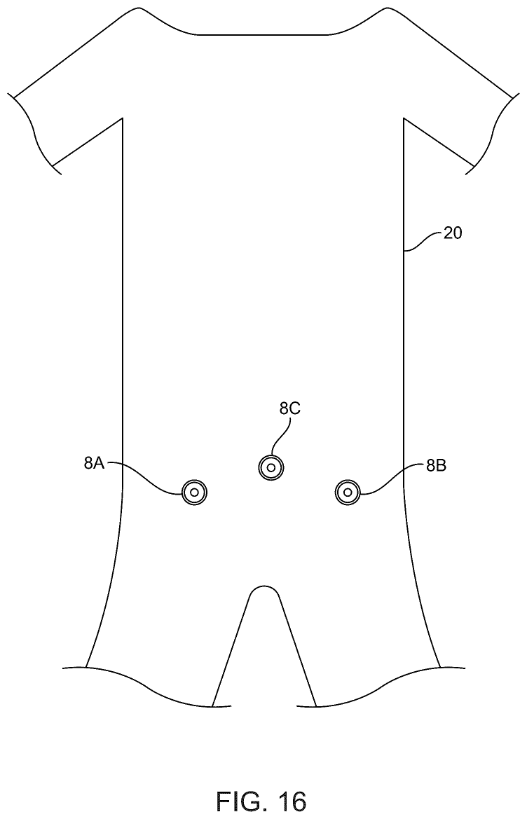

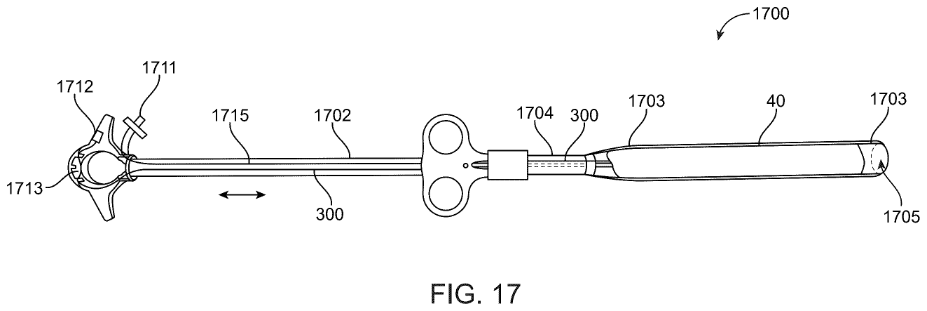

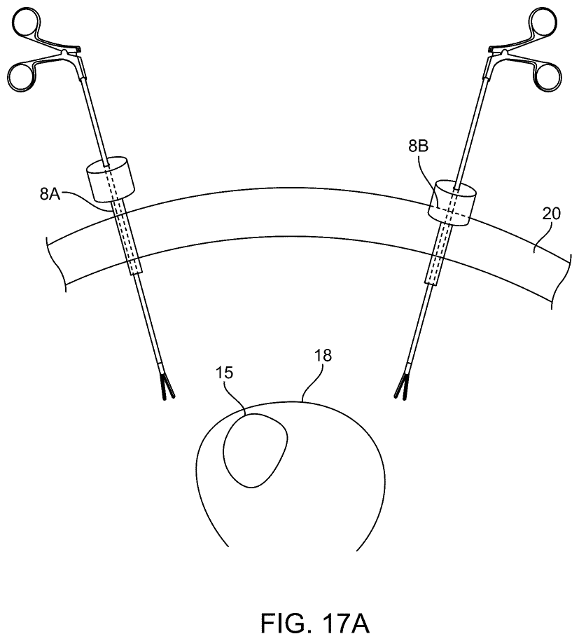

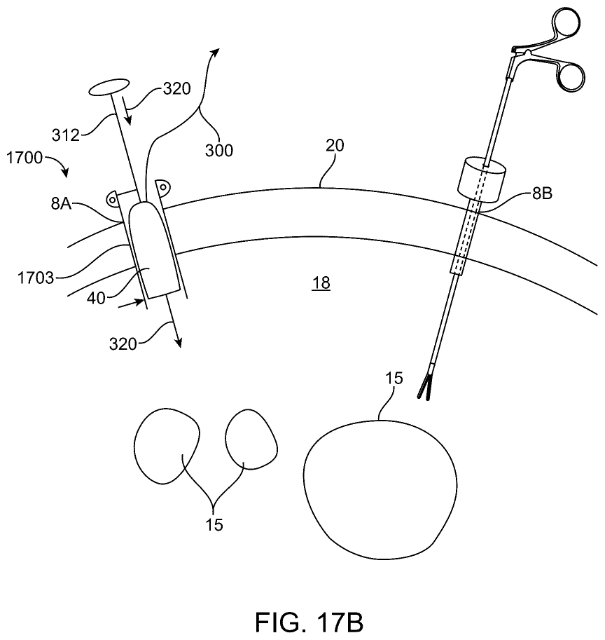

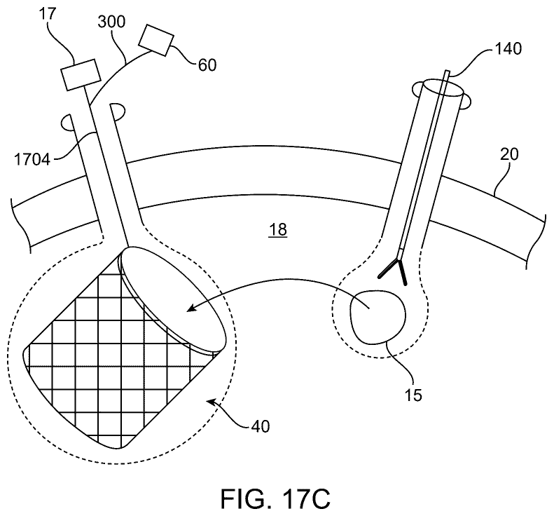

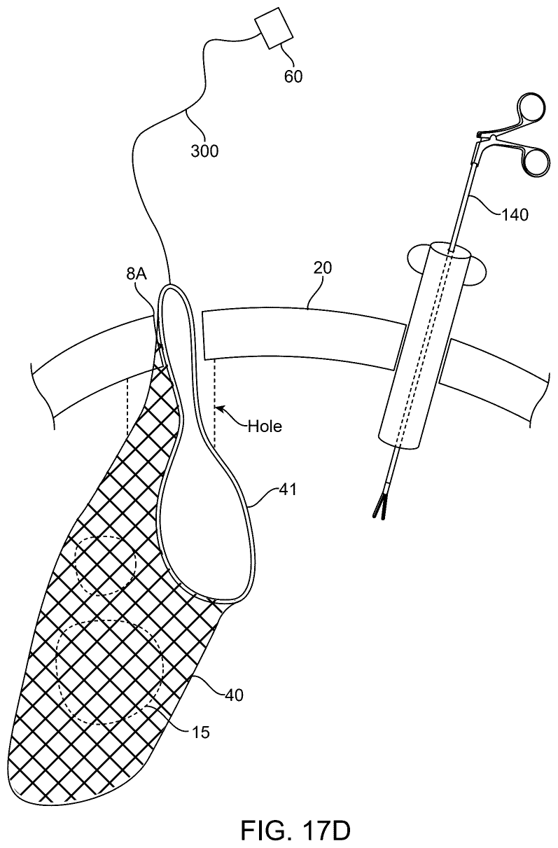

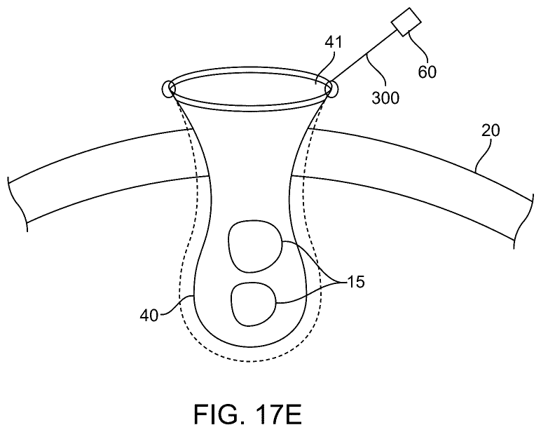

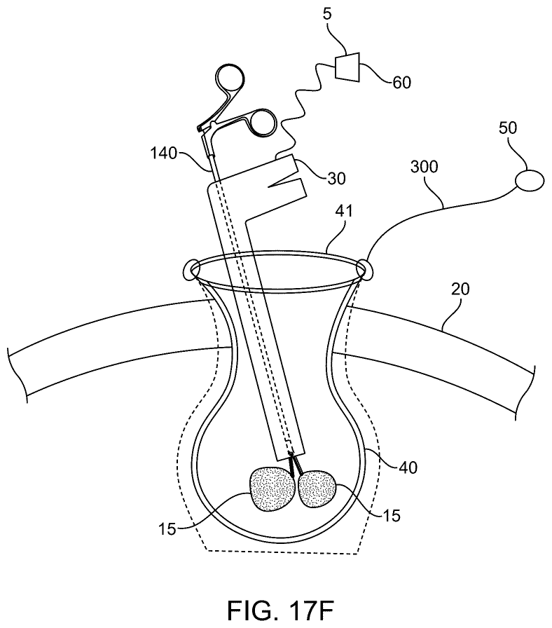

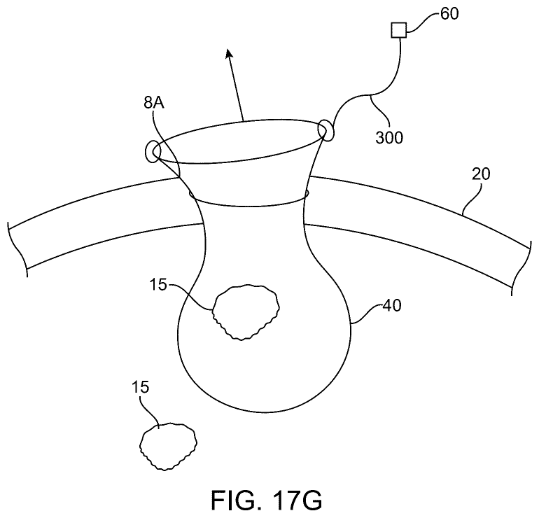

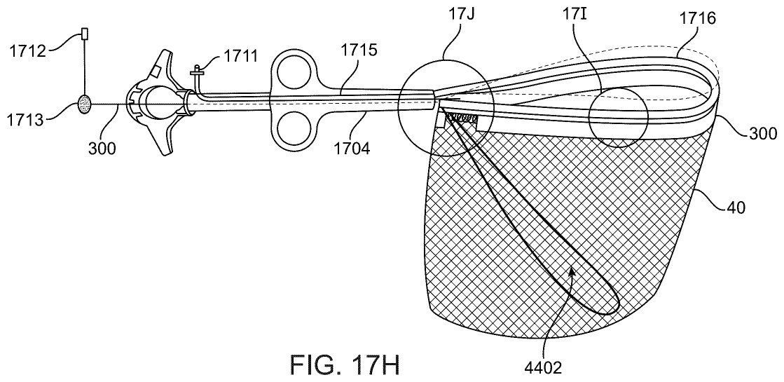

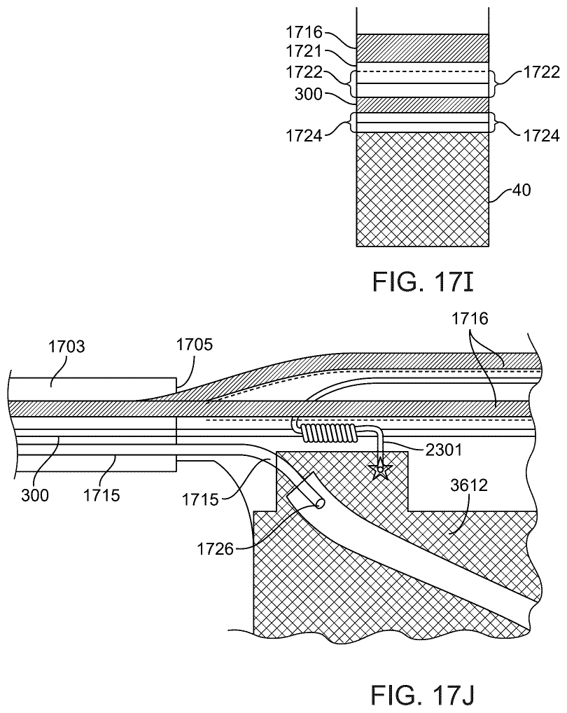

FIG. 16 shows an abdominal region of a patient 20 including incisions for performing a laparoscopic procedure in accordance with some embodiments of the present disclosure. FIGS. 17-17G illustrate an embodiment of a tissue container deployment device 1700 and a tissue containment method sequence using the tissue container deployment device 1700 in a transabdominal format on the patient 20 shown in FIG. 16. The tissue container deployment device 1700 may include components used for deploying a tissue container 40 and isolating a tissue specimen 15. The tissue container deployment device 1700 may be used with associated device and method embodiments for removing a morcellated tissue specimen 15 from the position 18 within the patient's body 20. In particular, the tissue container deployment device 1700 may be configured to contain and isolate a tissue specimen 15 in situ within a body cavity 18 of the patient's body 20, and an associated bulk tissue reducer 30 may then be used to reduce or otherwise morcellate the tissue specimen 15 while the tissue specimen 15 remains disposed within the patient's body 20 and isolated from surrounding tissue within the body cavity 18.

As shown in FIG. 16, at least one entry port is made into the body 20 for the device can be through an incision in the abdomen to provide at least one access point to the body 20 and/or abdominal cavity 18. For example, entry ports 8A, 8B, and 8C (generally, 8) can be made for use by instruments 140 for manipulation of the tissue specimen 15 and/or tissue container 40, such as graspers, tenacula, trocars, cameras and the like, all of which may be inserted into the body cavity 18 via the abdomen through small minimally invasive incisions in the patient's skin and underlying fascia.

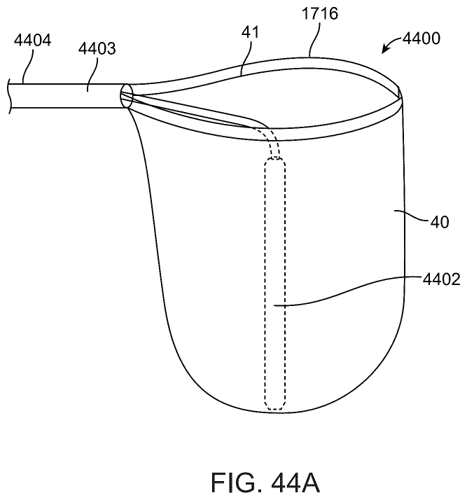

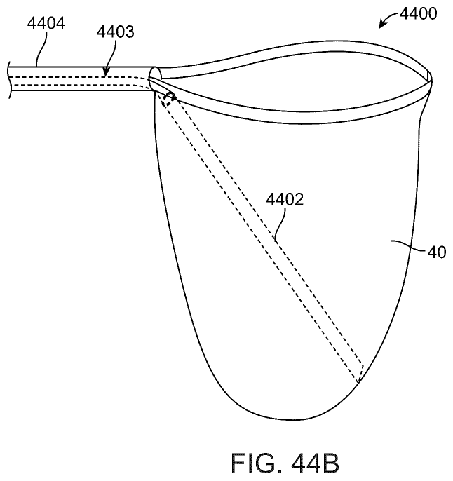

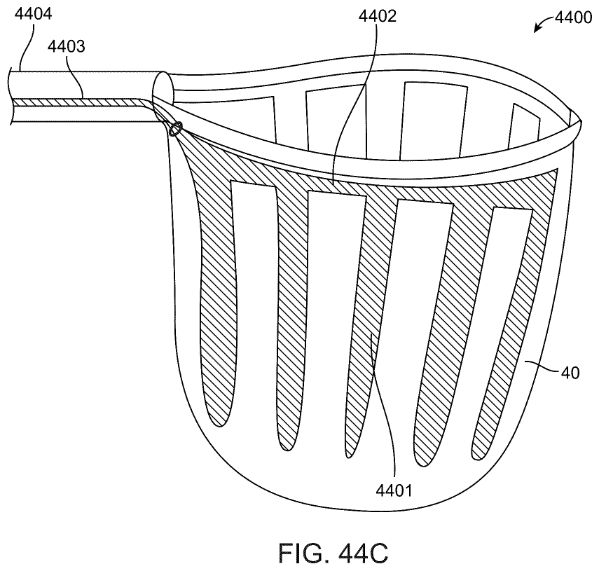

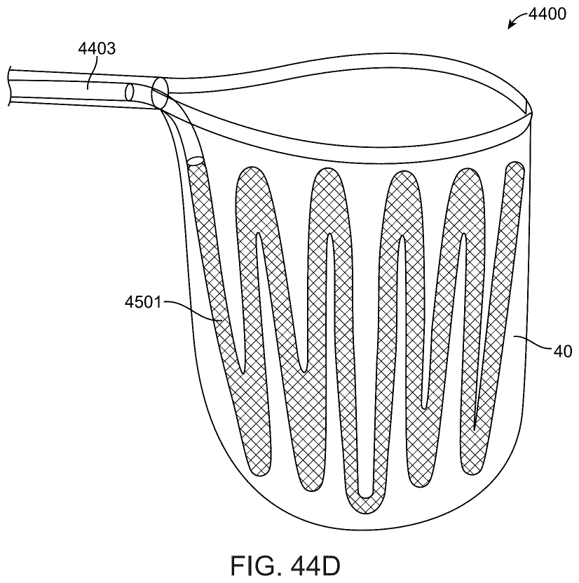

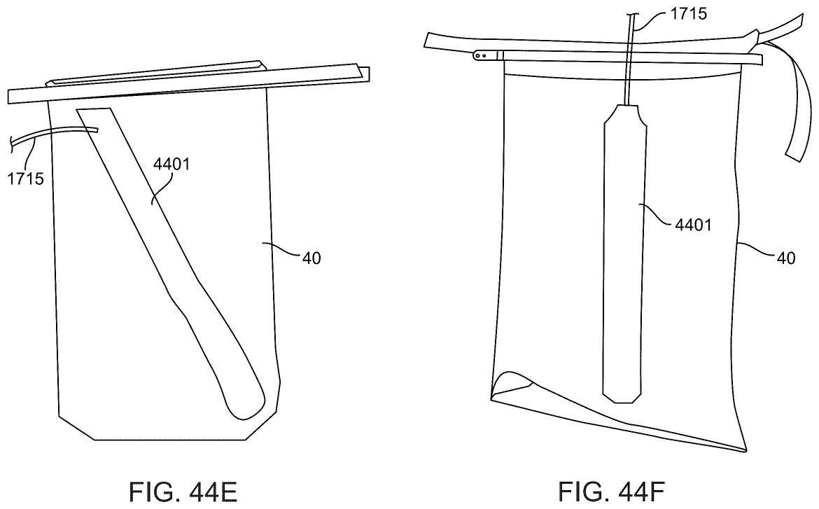

FIG. 17 shows a tissue container deployment device 1700 where a tissue container 40 is in a contracted storage stage prior to introducing the tissue container 40 into the abdomen, opening, and unfurling the tissue container 40. In some embodiments, the device 1700 may be part of the tissue containment and removal system embodiments 10 discussed herein. The device 1700, and more specifically, the housing or tube 1703 of the device 1700 in which the furled container 40 is stored, may have a dimension suitable for direct insertion through an incision or port 8A into the body cavity 18. For example, in some cases, the tissue container deployment device 1700 may be about 8 cm to about 12 cm long, more specifically, about 10 cm long and about 2 cm to about 3 cm wide, more specifically, about 2.5 cm wide. The tissue container 40 used within the tissue container deployment device 1700 may be the same as or similar to other tissue container embodiments 40 discussed herein. For example, such a tissue container used with the current device 1700 may include a tissue container 40 having a conductive layer or conductive elements 41 described with reference to the embodiments shown in FIGS. 8-10 and/or an inflatable strip or mechanism 4401, 4402 as discussed with reference to the embodiments shown in FIGS. 44A-44F, and/or other configurations and features of tissue container embodiments 40 discussed herein.

In some embodiments, the device 1700 may be laminated according to the method shown in FIGS. 17A-17G. The apparatus 1700 provides an initial inflatable strip that may be used to unroll or otherwise unfurl the tissue container 40. For such embodiments, the tissue container 40 may be rolled up in a tube 1703 along with an inflatable strip (not shown) coupled to a sidewall of the container 40 and configured to receive a source of fluid used for inflation an unfurling, unfolding, or opening of the container 40, which may operate in the same or similar manner as those embodiments described with respect to FIGS. 44A-44F.

An applicator 1702 may extend from a deployable laparoscopic introducer mechanism 1704 constructed as a handle or the like to apply a force against the unfurled container 40 to remove the container from an opening 1705 at the distal end of the tube 1703. The applicator 1702 may be movable, i.e., move linear with respect to the lumen of the handle 1704. Although not shown, the apparatus 1700 may have a button that is part of the applicator 1702 or at the handle 1704, e.g., at the proximal end in communication with the applicator 1702 that detaches the container 40 from the handle 1704, leaves the tether 300 coupled to the container 40, and allows a user to pull out the introducer or handle 1704 and then simultaneously cinch the bag closed with the tether and then slowly pull on the tether until the rim or edge of the container is pulled out of the abdomen.

The apparatus 1700 may include a lumen in the handle 1704 for receiving a length of the tether 300, which may extend to a container plug or connector 1712, which in turn can be configured for an electrical connection with the console 60. In some embodiments, the proximal end of the handle 1704 may include a male or female Luer™ connector 1711 or the like for connecting with a pressurized fluid source for providing pressurized saline, air, or other liquid or gas via an air tube 1715 (see FIG. 17H) to an inflatable member 4402 coupled to the container 40, shown for example, in the tissue container embodiment 40 of FIG. 44. The apparatus 1700 can also include a tissue container detachment tab 1713 that may be coupled to a string, wire, tether or the like extending through the handle 1704 to a metal band 1716 or the like for separating the container 40 from the distal end 1705 of the tube 1703, for example, during the procedure described in FIGS. 17A-17G.

As shown in FIG. 17A, a specimen can be identified. In doing so, graspers and/or other instruments inserted though the ports 8 can be used to detach the specimen, for example, fibroid or other body tissue specimen of interest, for subsequent removal and placement into the container 40. As shown in FIG. 17B, the tissue container 40 may be inserted into the body cavity 18 of the patient 20 by a rigid handle or the like as indicated by arrow 320. The container 40 here is in an undeployed state, for example, shown in FIG. 17. In some embodiments, a tether 300, for example, described in FIGS. 11-11D, has a distal end thereof secured to the rim 41 of the tissue container 40, for example, described in FIGS. 33-36. The distal end of the tether 300 coupled to the rim 41 or container in general can be inserted into the patient via port 8A (or any other port with or without a trocar or related intervening apparatus) with the container 40 by a rigid handle 1704 or related elongated object 312. For such embodiments, a container connector 411-418 shown in FIGS. 15A-15H, respectively, may be operatively coupled to a corresponding plug or the like at the proximal end 322 of the tether 300 to ensure that the correct container is used. The handle 1704 is used for inserting the container in a closed, deflated, wrapped, or undeployed state through a trocar extending through one of the ports 8A to a body cavity of interest.

As shown by the arrows in FIG. 17, the handle 1704 can move relative to the tube 1703. The handle can 1704 be activated by a button (not shown in FIG. 17 but shown in the FIG. 17H embodiment) in communication with the applicator 1702. Alternatively, the handle 1704 can insert the container directly laparoscopically, for example, through an incision in the skin without a trocar, e.g., as shown in FIG. 17B. Not requiring a trocar offers advantages, in particular, there is no need to swap the trocar back in after the bag is deployed in order to deploy the grasper to grasp the container rim 41, for example, shown in FIG. 17C. The rigid handle or related elongated object 312 along with another grasper (through port 8B) can be used to manipulate and position the container 40 for deployment to capture the specimen 15. Also, this feature eliminates the need to reinsert the trocar at port 8A through the same incision where the electronic cable (i.e., the tether 300) is also inserted through. In some embodiments, the elongated object 312 can be detached from the container 40, leaving the tether 300, and allowing a user to pull out the object 312 and the tether 300, followed by pulling out the bag ring 41. Accordingly, one port 8A is available for receiving the container, the bulk tissue reducer or other tissue reducing apparatuses, the tether and/or any electronic communication devices. Port 8B is available for receiving a grasper or other tool for loading tissue into the bag, or a camera or other supporting surgical instruments. Port 8C is typically reserved for the camera or other sensor(s).

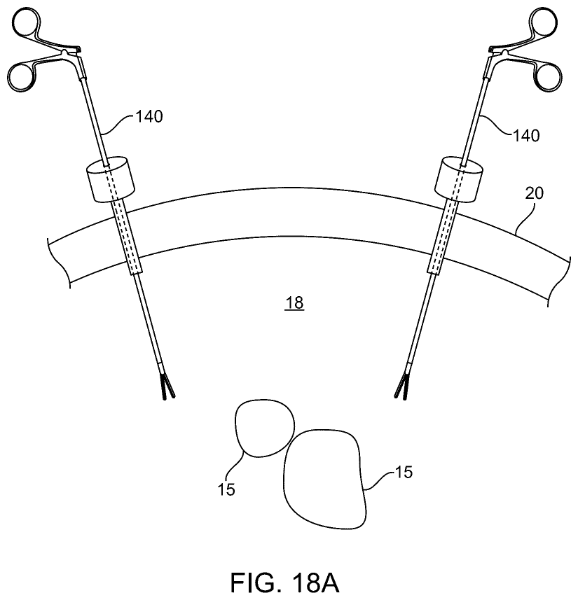

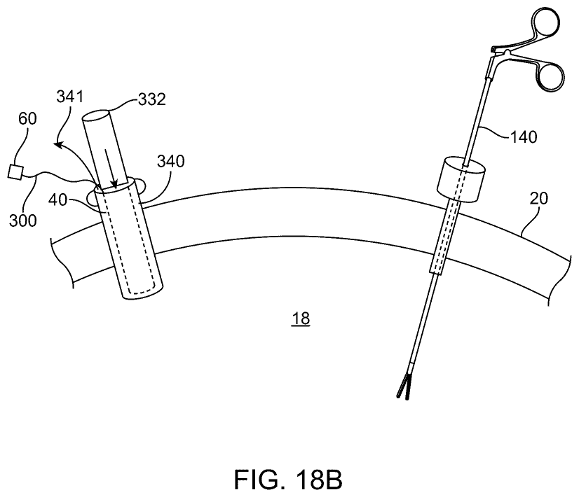

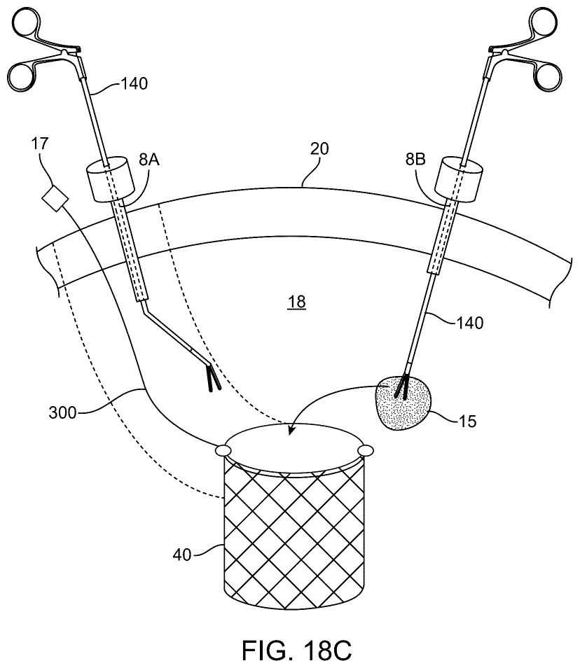

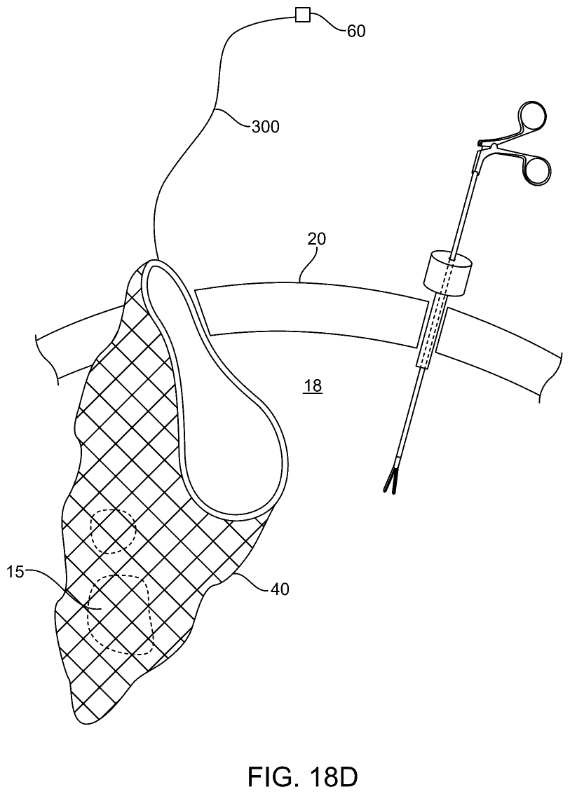

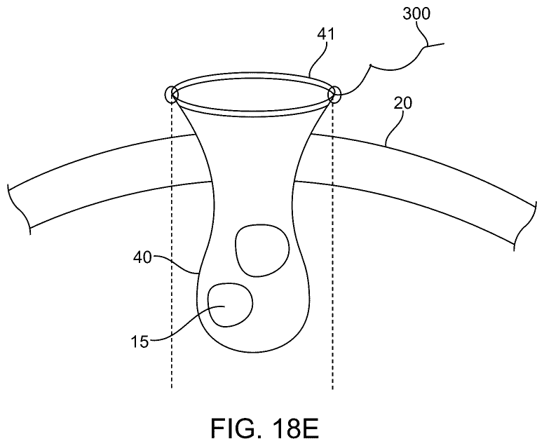

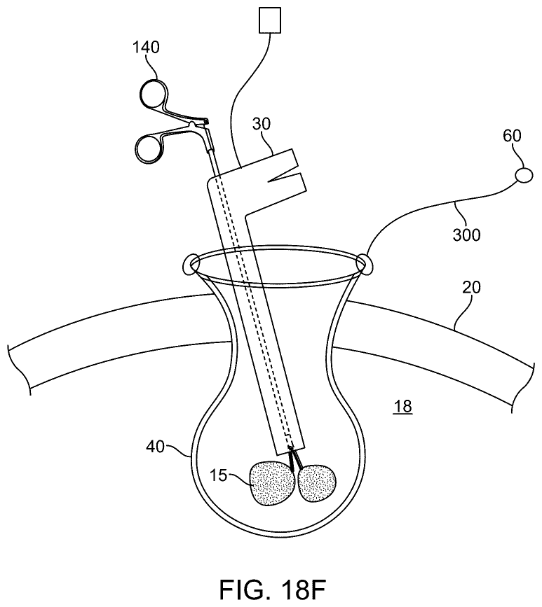

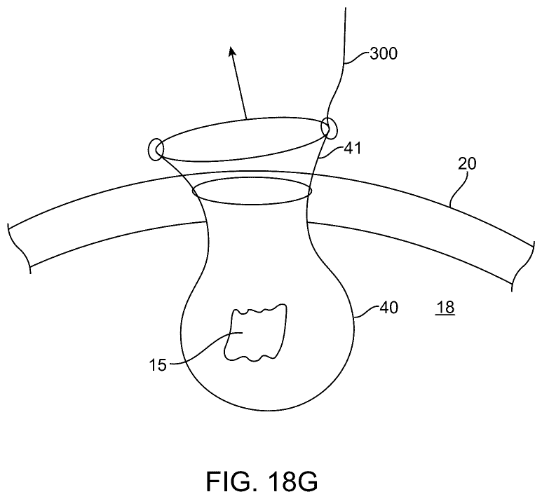

For the transabdominal procedure embodiments shown in FIGS. 16-17G and later in FIGS. 18A-18G, a bulk tissue reducer having a tissue cutter with a diameter of about 3 mm to about 25 mm may be used as discussed above. For such bulk tissue reducer embodiments, the associated access port 8A disposed in the patient's abdomen may be sized accordingly. In some cases, such an access port 8A may have a diameter of about 5 mm to about 30 mm.

After the specimen or multiple specimens are captured as shown in FIG. 17D, the container 40 is detached from the elongated object or handle (not shown), the elongated handle is removed. As shown in FIG. 17D, the tether or cable 300 is pulled slowly until the rim 41 of the container 40 can be grasped by the surgeon's finger (not shown). Then the rim 41 of the container 40 is continued to be pulled and is externalized out of the body. Alternatively the container can be cinched shut within the abdomen and then the rim can be externalized while the rim is in a bunched condition (not shown). Once the tissue specimen 15 is disposed within the interior volume of the tissue container 40, the rim 41 disposed about the body may be proximally withdrawn from within the body cavity 18 to a position outside the patient's body 20 while the tissue specimen 15 simultaneously remains within the interior volume and within the patient's body cavity 18, shown in FIG. 17E. This arrangement of the tissue specimen 15, tissue container 40 and body opening via port 8 effectively contains the tissue specimen 15 of interest in the interior volume of the tissue container 40 and isolates the tissue specimen 15 from surrounding tissues of the patient's body 20 disposed outside the tissue container 40. If there are any malignant cells, the cells are now environmentally contained and not exposed to the interior of the body. Additionally, the container protects the body's internal structures and organs from iatrogenic injury from the bulk tissue reducer or any other instrument that is inside the container.

As shown in FIG. 17F, a laparoscopic procedure can be performed to remove the specimen 15 from the body 20 via the incision 8 in which a tissue specimen is removed via a small incision using specialized tools are well known. In some embodiments, a morcellation procedure is performed, in which the tissue specimen 15 is cut or processed into pieces while still inside the patient, or at the level of the skin, or just outside the patient, so that they may be more readily removed. The tether 300 may be coupled to the console 60, for example, to apply a contact detection system (not shown) for detecting contact between the tissue removal instrument and the wall of the container 60, for example, described in international patent application no. PCT/US20/46135, incorporated by reference above. As shown in FIG. 17G, the container 40 is extracted through the incision forming the port 8A from the body 20. The incision can be closed according to a well-known suturing technique or the like.

FIG. 17H shows another embodiment of a tissue containment and removal system. FIG. 17H shows a side profile of an embodiment with a laparoscopic introducer mechanism 1704 with stiff bag hoop 1716 to aid in specimen loading, with conductive and non-conductive composite material (e.g., shown in FIGS. 8-10) that can be used with tissue cutters 30 with an auto-shutoff system, described herein, along with a tether cable 300 that has both mechanical and electrical capabilities, for example, shown in FIG. 17J, as well as an inflatable mechanism 1711, 1715 to aid in the unfolding of the container 4402. Here, an inflatable actuator mechanism can be used to unfurl the container, similar to FIG. 17. In doing so, the tube 1703 of FIG. 17 may extend from the introducer 1704, or as shown in FIG. 17H, the introducer 1704 is sufficiently elongated to include the tube (not shown in FIG. 17) and receive the container 40 until it is ready for deployment. In particular, the hoop 1716 along with the container 40 and some or all of the tether 300 may be movably inserted into the tube, which may form a lumen of the introducer 1704, prior to deployment.

The cable 300 with electrical-mechanical characteristics can be plugged into a console 60 or cutter handpiece. A tube 1715 connected to the inflatable mechanism can be connected to a saline syringe via a Luer™ connector or another fluid connection 1711. To deploy the container 40, the user pushes the end of the deployer or applicator through the cannula and the container 40 expands into the patient's abdomen. Once the container 40 has received a source of tissue 15, the applicator and rim can be extracted while simultaneously sinching and closing the container within the abdomen. This happens because the user can initiate this cinching or closing action by pulling, either simultaneously or close to simultaneously, on the tether 300 while retracting the stiffer hoop 1716 back into the introducer. Finally, as in the steps previously disclosed, the electrical tether/cable 300 can be pulled until the rim or edge of the container 40 is extracted.

FIG. 17I shows a blowup view of an embodiment of the tissue containment and removal system of FIG. 17H. Specifically, this embodiment shows how the stiff bag hoop 1716 interacts with the composite 40 and tether 300. In this embodiment the electrical and mechanical tether 300 is allowed to slip with respect to the container 40 as it is captured between two non-conductive layers. Alternatively, the electrical and mechanical tether 300 can be attached to the container 40 through loops or hoops (not shown but illustrated as tabs or loops 3616 in FIGS. 33-36) allowing the tether 300 to move with respect to the container 40. Alternatively, the electrical and mechanical tether 300 can rigidly be attached to the container 40 and not able to move with respect to the container. In one embodiment a perforated section 1721 of the container 40 allows for the container 40 to detach from the applicator and stiff bag hoop 1716 if desired. In another embodiment (not shown) the stiff members are not a connected hoop, but rather two members that slip inside a sleeve within the container 40, e.g., shown in FIG. 17I as the region between two plastic layers 1722, 1724. When the stiff members 1716 and introducer 1704 need to be removed, they are separated from the container 40 and exit the body while leaving the container 40 and tether 300 in the body. At this point in time the container 40 can be cinched closed using the tether 300 and a portion of the edge can be pulled out of the body, which will then enable the user to extract the full rim of the container.

FIG. 17J is a blowup detailed view of the combination of electrical, mechanical, and fluid connections of a tissue containment and removal system of FIGS. 17H and I. This blowup and close up view of the connections shows how the tether 300 can slip within the container tether sleeve to cinch the container closed while simultaneously detaching from the stiffer hoop 1716. The user can initiate this sinching or closing action by pulling, either simultaneously or close to simultaneously, on the tether while retracting the stiffer hoop back into the deployment cannula of the introducer 1704. FIG. 17J shows a slip knot 2301 which is stationary while the tether slips through it forming a loop that can be slipped closed while pulling on the end of the tether 300. In this embodiment the electrical connection hooks up to the conductive non-conductive composite through one connection point, but this system could also contain multiple electrical connection points (not shown). A slip knot 2301 is one type of knot but other knots or mechanisms can be used to allow one side of the cable to pull out of the bag while one end of the cable stays stationary with respect to the container 40.

FIG. 17J also shows how a fluid tube 1715 connects to an inflation mechanism 1726 which is on the outside of the container body. It should be noted that there are many different combinations of possible arrangements for the inflation mechanisms as shown in FIGS. 44A-44F. Also not shown is an embodiment where the inflation mechanism 1726 is down the side of the multilayer composite container not on top of the conductive/non-conductive layers. This embodiment would enable the inflation mechanism to be fabricated as a part of the seam, which already requires a sealing process. As shown in FIG. 17J, in some embodiments the still hoop 1716 or container rim 41, and the tether 300 are separated from the multilayer laminate. Additional embodiments could have those elements integrated together.

At least some of the components of the tissue containment and removal system of FIGS. 17H-17J are similar to or the same as those of the system of FIG. 17 and are not repeated for brevity. Other components in FIGS. 17H-17J are not shown in FIG. 17 but nevertheless equally apply. FIGS. 17H-J illustrate a combination of electromechanical cable 300, multilayer conductive/insulative composite forming the container 40, an inflation system, and fluid/balloon, a coupling to a morcellator or related tissue cutter 30 with auto-shutoff connectors.

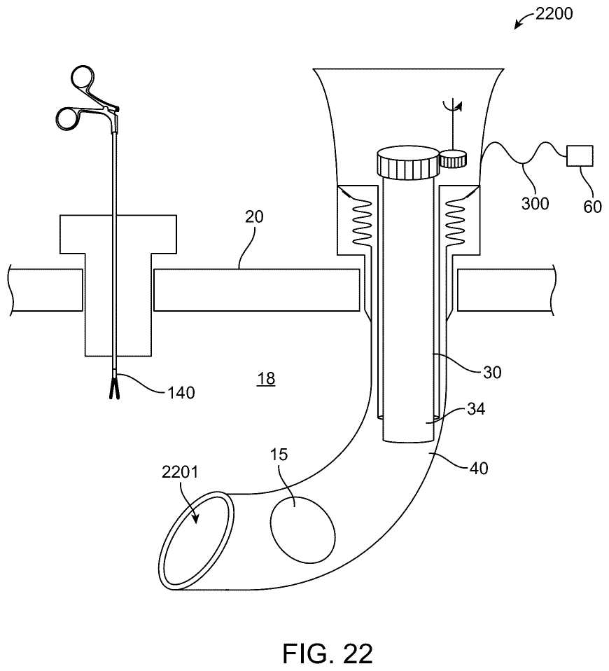

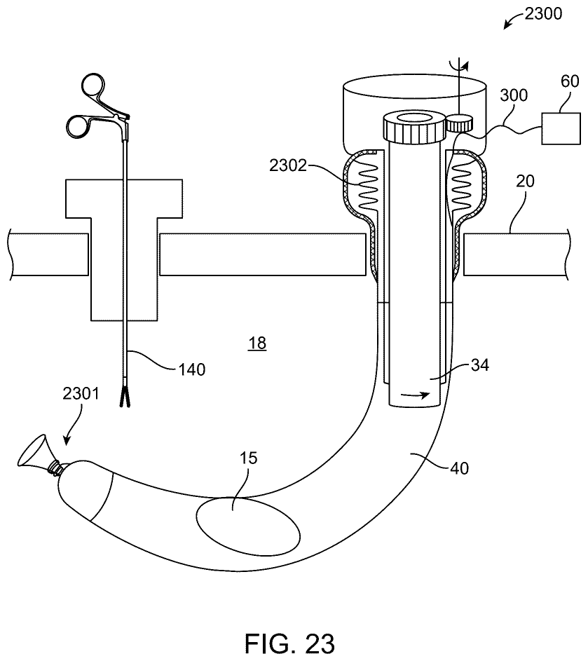

Referring again to FIG. 17J the hoop, referred to as a band 1716, extends from the distal end of the handle 1704. The band 1716 may be formed of metal and/or polymer composite such as plastic. In some embodiments, the band 1716 is flexible and can be moved by an actuator, e.g., 1702 in FIG. 17 into and out of the lumen of the handle 1704. The band 1716 may be shaped as a hoop or ring or may have two hemispheric portions extending from the handle, where a gap may exist between the distal ends of each portion. A seal 1722, for example, formed of a bilayer urethane, may provide a coupling mechanism between the band 1716 and the container cable 300. A perforated plastic layer 1721 may extend between the seal 1722 and band 1716. The perforation between the band 1716 and cable 300 permits the container 40 and attached cable 300 to be removed as a single unit from the band 1716 during a medical procedure through a lumen in a patient. Another bilayer urethane seal 1724, or other material seal, may be under the cable 300 so that the cable is sandwiched between seal layers 1722 and 1724. The container body 40 may be attached to the second seal 1724. The cable 300 may include a sleeve movable between two plastic layers 1722, 1724 for tightening the container 40, similar to a noose by way of a knot 2301 or the like, for example, shown in FIG. 23. The end of the noose can provide an electrical connection to the conductive layer of the container 40.

FIGS. 18A-18G illustrate a tissue containment method sequence of another tissue containment and removal system embodiment being used in a transabdominal format. Prior to performing the method sequence, at least one entry port is made into the body (similar to FIG. 17A) and as shown in FIG. 18A. A specimen 15 may be identified, similar to the procedure illustrated in the embodiment of FIG. 17B and as shown in FIG. 18A. As shown in FIG. 18B, an applicator 340 having a plunger 332 is either placed through a trocar or port or alternatively is placed directly into the body cavity 18 via an incision into the patient's body cavity 18. The applicator 340 may use the plunger to inject the tissue container 40 in a closed, deflated, wrapped, or undeployed state through the trocar extending through one of the ports into the body cavity 18. FIGS. 18A-18G, like FIGS. 17-17J, can include tissue container deployment device embodiments which have a tube and applicator configuration wherein the applicator is configured to slide within the tube and act as a plunger to deploy the collapsed tissue container 40 into the patient's body cavity 18. In some embodiments, the apparatus in FIGS. 18A-18G may be constructed to include a handle, such as handle 1704.

As shown in FIG. 18C, the first grasper and/or trocar 140 (in the first port 8A) may be reinserted after the applicator 340 is removed in FIG. 18B, shown by arrow 341 in FIG. 18B. In some cases, the first grasper 140 may be configured to articulate at a point proximal of the distal end thereof in order to facilitate positioning of the distal end of the grasper 140. Another grasper 140 disposed through port 8B may be used to capture the specimen 15 and place the tissue specimen 15 into the interior volume 42 of the tissue container 40 while the other grasper at port 8A can contribute to the procedure by also grasping the container 40. Since the tissue container 40 for this procedure embodiment is disposed within the patient's body cavity 18 in a “free floating” arrangement, two grasping devices 140 disposed through ports 8A and 8B, respectively, may be desired in order to maintain control of the movement of the tissue specimen 15. In FIG. 18D, the tether 300 is pulled until the rim can be grabbed by the surgeon and externalized. Alternatively, the container can be constructed such that by pulling the tether 300 the container is cinched shut and then the rim is externalized.

The embodiments of FIGS. 16-18G can be combined with containers with electrically conductive layers which are then connected via tether or cable 300 to the bulk tissue reducer 30 directly or connected through the console and then connected to the bulk tissue reducer or morcellator. These electrically conducive layers are required to activate an “auto-shutoff” feature as described with respect to FIGS. 3 and 25. Additionally, the tethers and cables 300 described FIGS. 17-18 can have both mechanical and electrical features as described in FIGS. 11A-11D. Finally, the embodiments of FIGS. 16-18G can also incorporate features that help unfold the electrically conductive containers either through inflatable balloons or other mechanisms, for example, described in FIGS. 44A-44F.

In both embodiments shown in FIGS. 17-18, the tissue container 40 is at atmospheric pressure and can be open to the air without insufflation of the abdomen. In another embodiment, the tissue container may be open to air for a period of time long enough to accomplish visualization of the target specimen before being sealed and pressurized to a different pressure than the atmosphere. Additionally, a microcontroller 17 (see, e.g., FIGS. 17C and 18C) may be attached to the container 40, the container tether 300, the container connector, or any part of the container 40 which can help the console 60 determine if the container 40 is an approved container or counterfeit, for example, part of the counterfeit check system 400 described with reference to FIGS. 12 and 13. Once the tissue specimen 15 is disposed within the interior volume of the tissue container 40, the rim 40 or edge in the absence of a rim may be withdrawn through the port 8A to a position outside the patient's body 20 as shown in FIGS. 18D and 18E. Thereafter, the tissue specimen 15 may be morcellated and withdrawn from the interior volume of the container 40 as shown in FIGS. 18F and discussed above with respect to FIG. 17F.

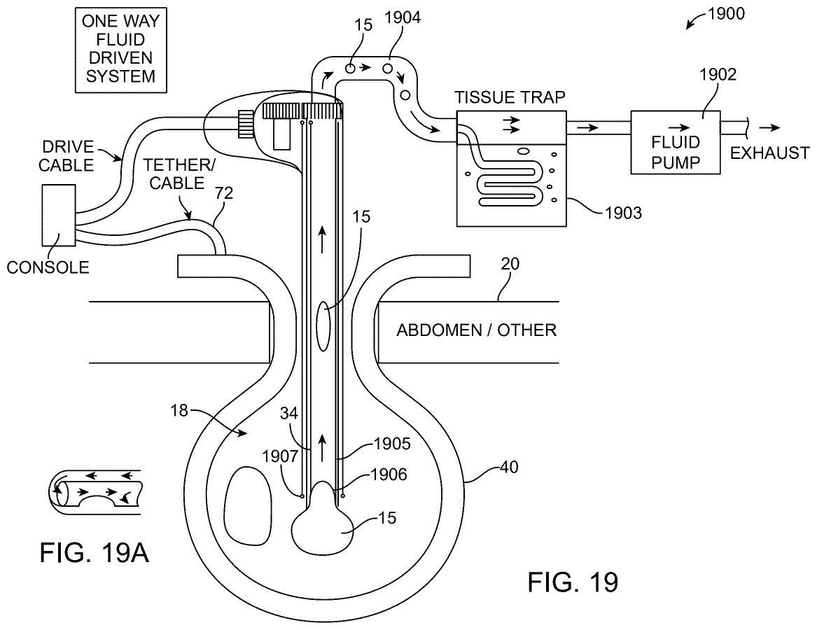

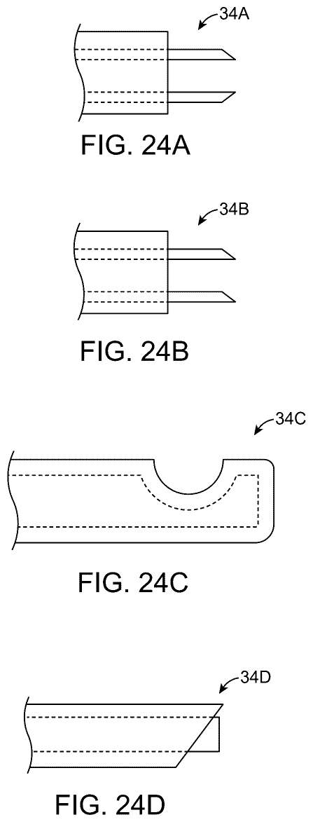

FIG. 19 illustrates a tissue containment and removal system embodiment 1900 where the tissue specimen 15 is pulled through a tissue cutter 34 due to a change in pressure or a force such as vacuum applied through a fluid. This may replace the need for a tenaculum or visualization during an operation in which intrauterine tissue or the like is removed from a patient's body. The fluid could be a liquid such as water or saline, or a gas such as air. Such a fluid (or combination of fluids) may be used pull the tissue specimen 15 through the cutter 34. FIG. 19 shows how a fluid pump 1902 pulls on the tissue 15 causing it to fall or otherwise be drawn into the blade 1906 of the cutter 34. Example cutters 34 are shown in FIGS. 24A-24D, but not limited thereto. The pump 1902 may include a peristaltic volume displacement pump, a reciprocating piston pump, a hydraulic pump, an axial fan or blower pump, or any other suitable type of fluid pump for liquids or gasses. In addition, in some cases, any other suitable source of negative pressure or vacuum may be used in place of pump 1902. If a liquid is used, the liquid could be loaded into the container 40 prior to suction and tissue cutting. The liquids are generally incompressible and therefore are good candidates for hydraulic pumps. Additionally, a gas such as air, CO2, or Nitrogen, for example, may be used to generate suction or a pressure gradient to pull the tissue 15 into the cutter 34. For example, a vacuum of 1000 to 1 mbar/760 to 0.75 Torr can be produced, but higher vacuums may equally apply, for example, 10⁻⁷ to 10⁻¹¹ mbar/7.5⁻⁷ to 7.5⁻¹¹ Torr.

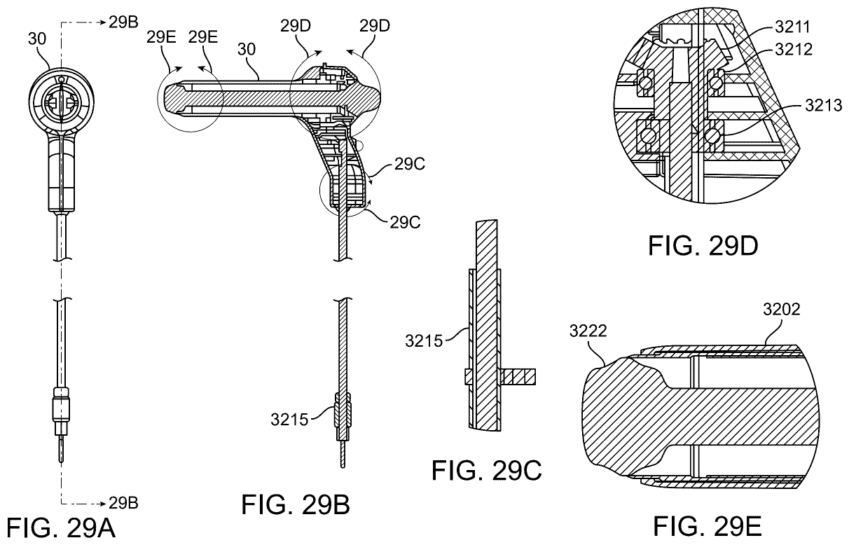

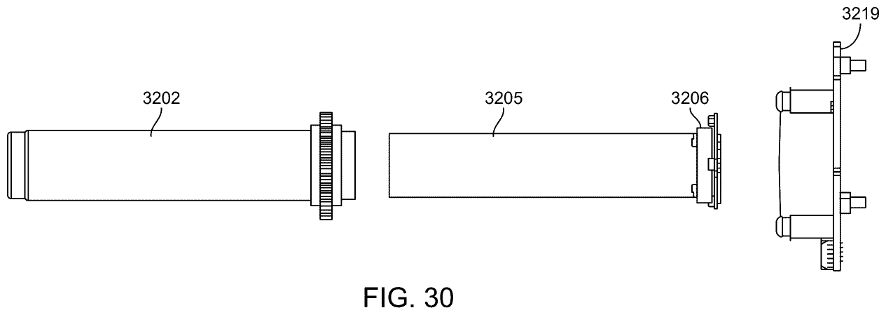

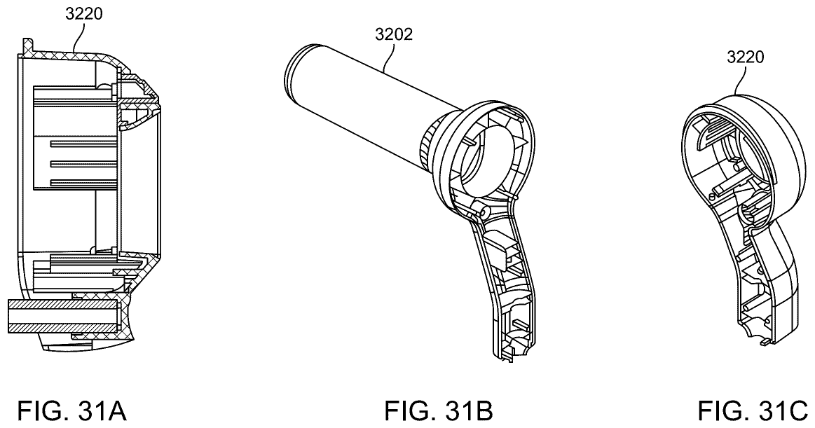

The cutter 34 may include a high friction, anti-rotation tube 1905 having an opening proximal the cutter blade 1906 for receiving the tissue specimen 15 in the body cavity 18. This high friction anti-rotation tube (described in detail with respect to the embodiment illustrated in FIGS. 28-32) increases the friction between the specimen and the tube and thus can require less suction force both to suck the tissue down the bore of the reducer, but also less suction force to keep the tissue stable against the rotating cutter. The anti-rotation tube, as described above, minimizes the surface area of the exposed blade to lower the forces required to pull the tissue through the blade and accomplish the cutting. For the embodiment shown in FIG. 19, the outflow of the pump 1902 may go to a containment location, or tissue trap 1903 where the tissue 15 can collect. The tissue pieces can be pulled through another tube 1904, or hose or the like between a proximal end of the cutter 34, which is sealed to provide an escape of liquid, gas, and/or tissue, and the tissue trap 1903. The entry port into the body for the cutting device 34 and/or complementary trocar or related device, can be through an incision in the abdomen, through the vagina or rectum or any number of access points to body and/or abdominal cavity 18.

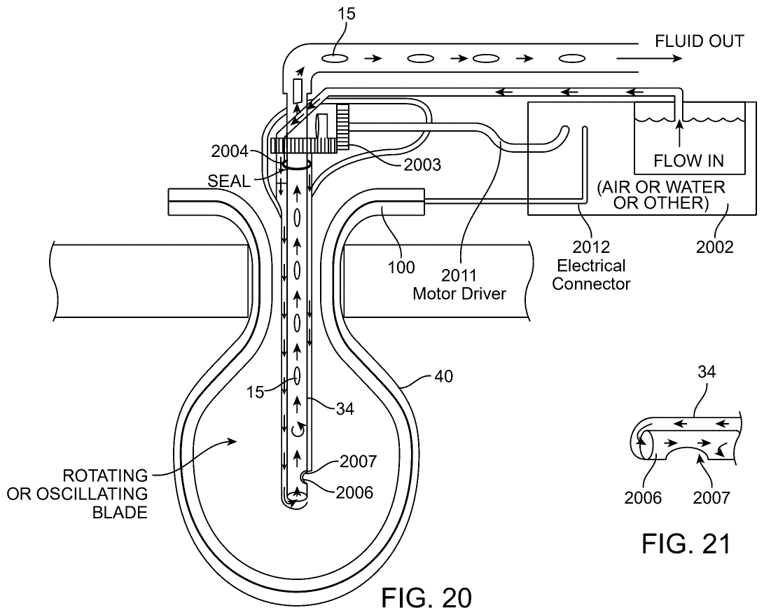

In FIG. 19, the pump 1902 creates a single flow path for the fluid (as shown by the arrows). In other embodiments, as shown in FIGS. 20 and 21, the instrument 34 may recirculate the fluid (air or liquid) by providing a circular loop of fluid flow to evacuate pieces of the tissue specimen 15. For example, the fluid, e.g., air or liquid, generated by a pump 2002 is recirculated down the instrument 34, passed through the blade 2006 (shown by the insert) and back out of the instrument 34. In some embodiments, the pump 2002 may communicate with the console 60 or be part of the console 60. The pump 2002 may have a motor driver 2011 for controlling the motor 2003 generating the forces for generating the fluid flow through the cutter 34. The pump 2002 may have an electrical connection 2012 for providing a source of power to the conductive strands of the wire mesh 100 of the container 40 (see for example, FIGS. 9 and 10). A seal 2004 may be about the proximal end of the cutter to prevent the escape of circulating fluid. A tissue solid trap location (not shown in FIG. 20) can be installed before the fluid pump 2002 to remove or collect any tissue fragments 15 that are evacuated. Since the fluid is flowing down the cutter 34 in this embodiment the cutter type that is shown in FIG. 20 is a rotating cutter, but not limited thereto. There is a hole, notch or exposed section 2007 in the rotating cutter 34 which allows tissue to be trapped between this rotating cutter and a sharp edge. The sharp blade 2006 could be the internal blade that is spinning but it also could be the external blade that is stationary.

FIG. 22 shows an embodiment of a tissue containment and removal system 2200 where the tissue container 40 is in the form of a sheath that slides down the length of the tissue cutter 30. This tissue container embodiment may be loaded with a specimen 15 through a hole or ring 2201 at the distal end, and then the end of the tissue container is sealed off through any of a variety of suitable devices and methods. The tissue container 40 may be supplied sterile and may be a consumable or disposable along with the tissue cutter. The container 40 can be sealed off in a number of ways once a tissue specimen has been disposed therein.

FIG. 23 shows an embodiment of a tissue containment and removal system 2300 which is configured such that the distal end of the tissue container 40 may simply be tied into a knot 2301 in some instances in order to seal the container. The container 40 may also be sealed with a thin suture cord or wire added by a surgeon, a zip tie, magnets, snaps, a heat seal, a chemical bond, or any other number of suitable sealing mechanisms. In addition, the distal end of the tissue container 40 (either sealed off or not sealed off) may be pulled out of the patient's body through another port and the end of the tissue container 40 may then be tied, sutured, heat-sealed or closed through any number of closing methods.

In some cases, the sheath like tissue container 40 may be housed such that an accordion-type section embodiment 2302 of the tissue container 40 may be convenient to configure, store, and deploy. Once the tissue specimen 15 is reduced by pulling tissue specimen 15 through the cutter 34 either via a tenaculum or suction, the entire system 2300 including the tissue container 40 may be pulled out of the patient's body 20. In some cases, the system 2300 may be inserted into the abdomen as is common in a mini-laparotomy procedure, or the system 2300 may be inserted in or through another orifice such as the vagina, the rectum, mouth, nose, etc. Conversely, once the tissue container 40 is tied off, for example, by an instrument 140 such as a grasper, the tissue container 40 may be retracted against the blade of the bulk tissue reducer 30, effectively pulling the tissue specimen 15 towards the stationary bulk tissue reducer 30. This can be performed by either manually pulling the tissue container 40 towards the cutter or by mechanically retracting the tissue container 40 back into the system 2300.

FIGS. 24A-24D show different embodiments of cutters 34A, 34B, 34C, 34D (generally, 34), respectively, that may be used with system embodiments as discussed above, for example, in connection with the anti-rotation tube. FIG. 24A shows a cutter 34A with a beveled edge as the outer diameter, FIG. 24B shows a cutter 34B with a beveled edge as an inner diameter. The cutter may also have a serrated edge (not shown), oscillating, rotating and/or reciprocal blades (not shown) or any number of other configurations. FIG. 24C shows a rotating cutter 34C that nibbles at the specimen from the side through a hole. For such embodiments, either the inner, outer or both of the components may be sharpened. FIG. 24D shows a cutter 34D having a cannula (e.g., 38 in FIG. 1) that is at an angle to the outer sheath.

Additional tissue containment and removal system and method embodiments are discussed below and may include any of the features, dimensions, and materials of any of the suitable embodiments discussed herein. The tissue containment and removal system shown in FIG. 25 may include embodiments directed to a breach-resistant tissue containment and removal system 2500 designed for hysterectomy. There are several safety features in the system, one of which is an embodiment of an auto-shutoff feature described with respect to FIG. 3. The system 2500 is composed of two main disposable components: a tissue container 40 and a bulk tissue reducer 30, for example, described in embodiments above. There are also two reusable secondary components: a tenaculum 45 or related instrument and a console 60 which powers the bulk tissue reducer 30. Although FIG. 25 describes the system 2500 in connection with a trans-vaginal procedure, the system 2500 can equally apply to other medical procedures such as a laparoscopic, e.g., trans-abdominal, procedure.

As described with regard to some embodiments herein, the container 40 can be deployed trans-vaginally to capture the uterus and any associated structures. Once the specimen has been captured, the open end of the tissue container 40 is guided out of the body via the vagina. In some embodiments, various safety features may apply. The first safety feature is an auto-shutoff feature. Here, by plugging the container 40 into the console 60, the container 40 becomes a “smart” container that can detect when the bulk tissue reducer 30 comes into contact with the container 40 and in doing so immediately stops the operation of the system. In particular, the cutting blade immediately stops rotating or otherwise moving to prevent damage to the wall of the container. The second safety feature is a durable steel cloth layer of the container 40 that is resistant to the cutter, for example, shown in FIG. 9. Clinically there is a strong need for a tissue containment and extraction system that is fool-proof and a container that is breach-resistant. There are many benefits of an easy-to-use, breach-resistant system. First, it can prevent the spread of malignant cells that could be contained in the tissue. Second, it can prevent iatrogenic injury of important organs and structures that need to be protected during surgery such as bowls, bladder, ureters, arteries etc. In addition, simplicity and accessibility can enable trouble free and convenient medical procedures to rapidly remove the tissue once it has been placed into the container. The auto-shutoff feature provides confidence that once the tissue specimen has been detached from the body and placed in the container, that a low risk, easy to perform extraction is the last step of the procedure.

The breach resistant features of the container 40 are achieved through a seamless communication between the bulk tissue reducer 30 and the container 40. And the enablement of the auto-shutoff feature via the container cable 72 shown in FIGS. 1-3, or the special-purpose tether 300 shown herein and the handpiece cable 56. The auto-shutoff feature has the following experience for the user: First, during active cutting, the feature enables the immediate and systematic shutoff of the cutting device 30, e.g., bulk tissue reducer or morcellator nearly immediately if it comes in contact with the conductive layer of the container 40 effectively preventing an undesirable container breach. Secondly, it alerts the physician that the motor has been stopped. Third, it disarms the system by automatically putting the system into a mode that prevents the surgeon from continuing with cutting until the system is reset. Finally, the system can be designed to be reset either through depressing a reset button 63, also referred to as a standby/ready button, shown in FIG. 6, or having the system automatically reset after a certain amount of time has transpired. The resulting user experience is one in which the physician wields a very sharp tool that can cut through uterine fibroids or tissue as hard as bone, but cannot physically breach the container with the prescribed and matched cutting tool, in any method that the physician attempts: 1) high force 2) quick motions 3) high angle of contact 4) low angle of contact 5) in a liquid, aqueous or blood filed environment 6) in a dry or air filled environment, 7) in a slippery environment, 8) in a low light environment, 9) against a hard object, 10) against a soft backed object or suspended in air/gas, or any other environmental condition that a physician may encounter in the operating room.

Some minimally invasive, laparoscopic, trans-vaginal, or trans-anal surgeries have the benefit of working space for A) visualization of the specimen through a laparoscope: 1) directly 2) through a clear or translucent container, or 3) from within a container or B) to avoid contact of the container with a cutting instrument such as a rotating blade (as in a morcellator or bulk tissue reducer), scalpel, or other instrument. However, many clinical procedures do not have the luxury of this working space and thus benefit tremendously from a system that enables cutting in very close proximity to the container. The auto-shutoff system of the embodiments discussed herein enables cutting in small or tight working spaces and in close proximity (0.1-100 mm) to the container itself. This is made possible because the auto-shutoff automatically shuts down the motor if the rotating blade cuts through the innocuous first insulative layer of the container, if one is present, and comes in contact with an electrically conductive layer or segment of the container. The auto-shutoff feature prevents a complete breach of the container because the conductive layer and second insulative layer remain intact.

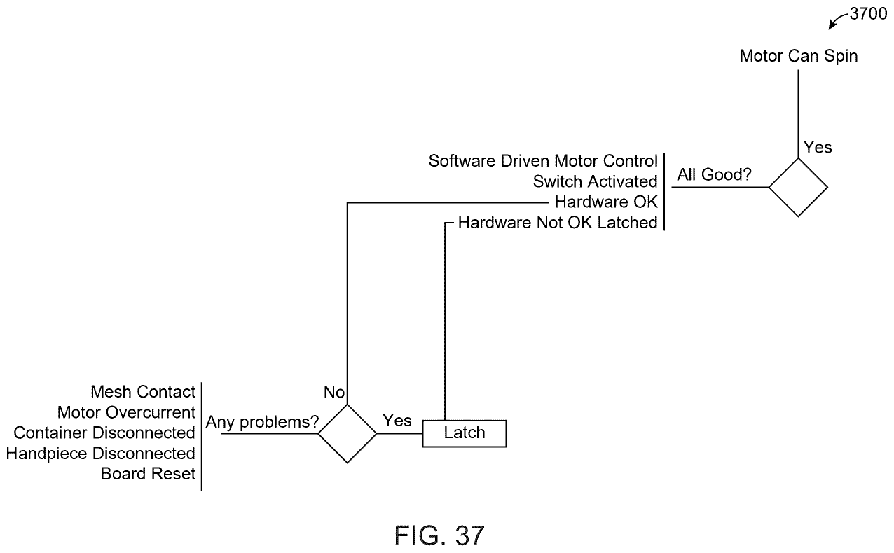

As shown in FIGS. 37-39, the auto-shutoff functionality can work by including electrical logic either in hardware, software, and/or firmware in the console 60 which is connected to by the bulk tissue reducer 30 and container 44. Alternatively the container 40 can plug directly into the handpiece (not shown) with the logic in the hardware, software, firmware housed in the bulk tissue reducer, container, or the connector itself.

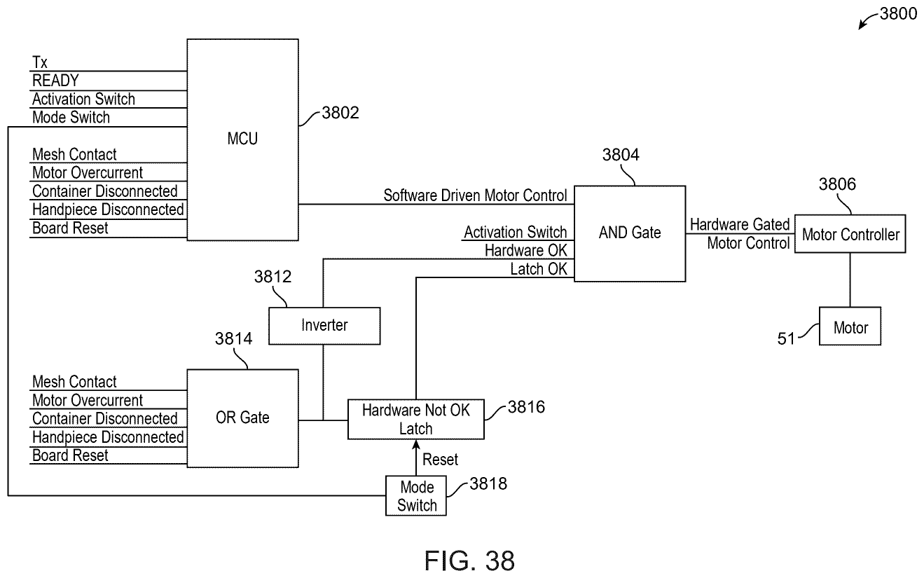

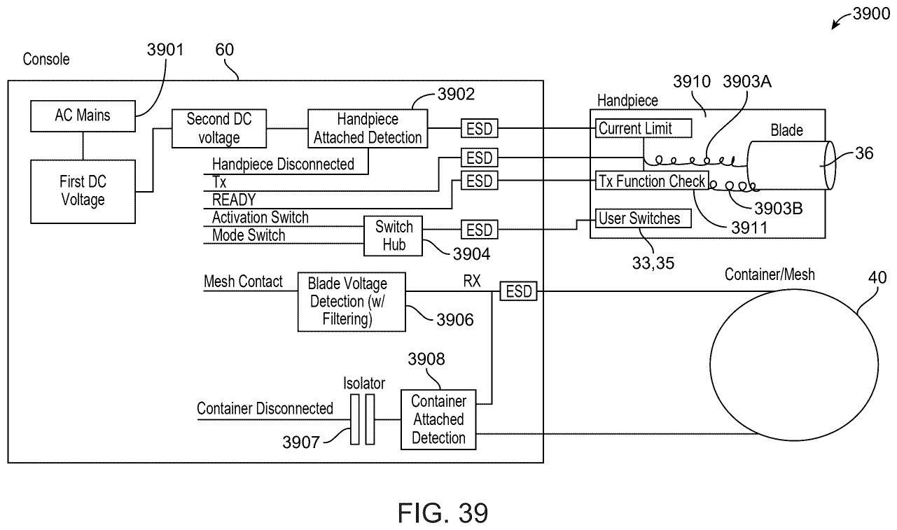

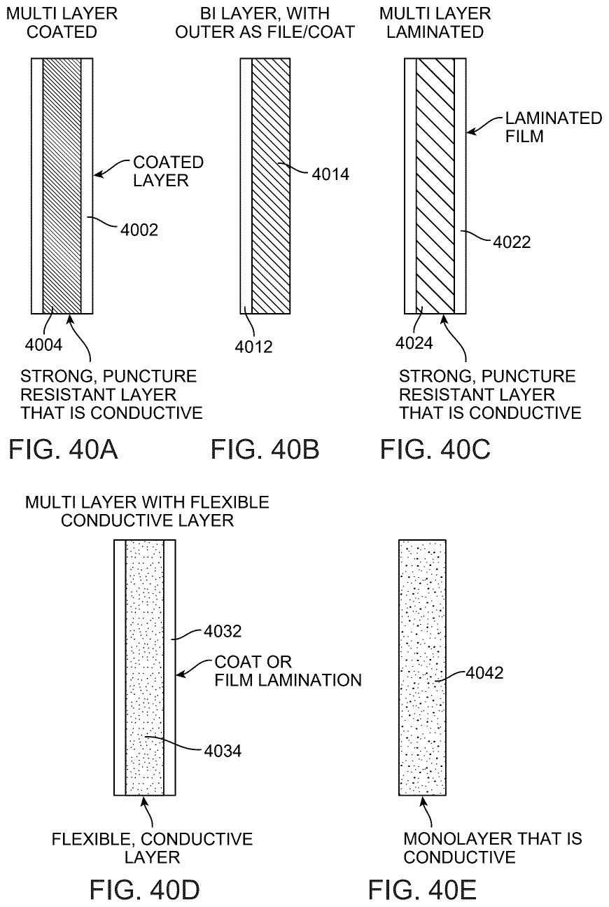

As shown in the logic, control, and hardware schematics of the auto-shutoff functionality described with reference to FIGS. 37-39, respectively, the first step in the auto-shutoff sequence is the blade contact with the conductive layer, for example, shown in FIG. 40. Then the connected circuit consists of the connectors to the container 40, the container cable 300, the star or spike washers 3612 (multiple for redundancy), the steel mesh or conductive layer of the container 40 then through the rotating blade 36, then into the spring contacts through the screws into the connector terminal through the handpiece cable 56, into the handpiece connector into the console 60. A source of current will flow from the voltage source in the cutter handpiece, through the container, and back into the console where the Blade Voltage Detection is shown in FIG. 39. The resulting mesh detection event goes to the OR gate in FIG. 38 then it is inverted and prevents the microcontroller (MCU) activation on the motor 51 at the AND gate. The result is the automatic shutoff of the motor, or “auto shutoff.” This “auto shutoff” occurs through the braking of the motor, which is very rapid, e.g., on the order of 10-50 ms but not limited thereto, thereby stopping of the cutting blade 36 before it can damage the container 40.

This motor brake can occur by shorting the two terminals of the motor across one another. The motor continues to be shorted or braked until instructed otherwise. In other embodiments the braking can occur by applying a constant reverse polarity or applying a reverse polarity through pulse width modulation. If the auto-shutoff system is triggered then the motor 51 will automatically be braked, an alarm will sound, and the system will automatically be disarmed. To re-arm the system, several elements are required as shown in FIG. 38. 1) There can be no blade-to-container contact as measured by the blade voltage detection circuit shown in FIG. 39, the cutter handpiece and container must be plugged in, and the standby/reset button must be depressed. In some embodiments, the auto shutoff system shown in FIGS. 37-39 can communicate with the console 60 wirelessly, e.g., via Bluetooth™ and without a console-container electrical cable 300 or a console-to-cutter cable.

The schematic in FIG. 37 describes the logic 3700 to allow the motor 51 (either in the bulk tissue reducer 30 (also referred to as a tissue cutter) or the console 60) to spin. For some embodiments, the motor 51 can spin only if all four conditions are met. The first condition is that the software-driven motor control signal is activated. The software-driven motor control signal is controlled by the software running on the MCU 3802 shown in FIG. 38. If the MCU 3802 outputs a digital signal greater than 0 V, the software-driven motor control signal is activated. The second condition is that the switch is activated. The switch activated signal is active if either of the activation switches 33, 35 is pressed. The third condition is that the hardware is operable. The hardware is OK only if all five conditions are met: there is no mesh contact detected, there is no motor overcurrent detected, the container is connected, the cutter handpiece is connected, and the board is not reset. Mesh contact occurs when the blade 36 contacts the conductive layer of the container 40. There is no mesh contact detected if the voltage measured by blade voltage detection circuit is above a threshold voltage, e.g., 1.0 V or nominally 20% of a source voltage ranging from 10% to 30%. A motor overcurrent event occurs when the current draw of the motor exceeds a predetermined current value. For example, a motor overcurrent signal is the output of a comparison between the motor current draw to a current threshold. If the voltage measured by the container attached detection circuit is below the threshold, the container is connected. If the current drawn by the bulk tissue reducer 30 (as measured by the Handpiece Attached Detection circuit) is above 30 mA, the cutter handpiece 30 is connected. If the 3.3 V supply is lower than expected, the board is reset, for example, described with reference to FIG. 38. The fourth condition is that the “hardware is not okay” latch is not activated. The “hardware not OK” latch is a circuit that stores information through two stable output states. If any of the hardware OK conditions are not met, then the “hardware not OK” latch is activated and locks its output signal so that it cannot change until the latch receives a reset signal.

In some embodiments, the container 40 may include a second conductive layer (not shown) proximal the exterior of the container. This permits the system to detect contact with the cutter blade if the blade penetrates the first conductive layer but also the non-conductive layers protecting the specimen inside the container 40. If the blade makes contact with the second conductive layer, this indicates that the container 40 has been breached by the blade and that there is a risk that the contents of the container 40, such as malignant cells, may leak from the container. Here, if such contact is made, the logic circuit can include a circuit that generates an alarm signal that can be sent to an alarm system that generates a sound, activates a light, and so on indicative of a warning of the possible container breach.

FIG. 38 illustrates a control schematic 3800 of the motor 51. The hardware logic circuit controls the motor controller 3806 which operates the motor 51. In some embodiments, the hardware logic circuit comprises an OR gate 3814, an inverter 3812, “a hardware Not OK latch” circuit 3816, and an AND gate 3804. Different or additional logic circuits may be used to perform the same functions. For example, the OR gate 3814 and AND gate 3804 may be replaced by an XOR gate or the like. The microcontroller unit (MCU) 3802 outputs a pulse-width modulation (PWM) signal, ranging from a 0% to 100% duty cycle, to the AND gate 3804 of the logic circuit. If all of the other AND gate inputs are satisfied, this signal is passed through to the motor controller 3806 which uses it to control the motor speed.

The Mesh Contact, Motor Overcurrent, Container Disconnected, Handpiece Disconnected, and Board Reset signals as inputs to the MCU 3802 all serve as input signals into both the hardware logic circuit and the MCU. The Mesh Contact signal indicates if the blade 36 is contacting the conductive layer of the container 40. The Motor Overcurrent signal indicates if the motor current draw exceeds a threshold current, for example, 3.0 A. The Container Disconnected signal indicates if the container 40 is connected if the voltage measured by the container attached detection circuit 3908 shown in FIG. 39 is above a threshold voltage, e.g., 0.45 V. The Handpiece Disconnected signal indicates if the bulk tissue reducer 30 is connected if the bulk tissue reducer current draw measured by the handpiece attached detection circuit 3902 of FIG. 39 is below the threshold current, 30 mA. The Board Reset Signal indicates that the voltage supply, e.g., 3.3 V supply is lower than expected.

As shown in FIG. 38, these input signals feed into hardware logic circuit 3800 first through the OR gate 3814 to determine if the hardware is in a condition for operability (“OK”). The output of the OR gate 3814 is then inverted and sent to the AND gate 3804 as the Hardware OK signal. The OR gate output is also passed to the “Hardware Not OK latch” circuit 3816 whose output is passed to the AND gate 3804. If the hardware is not okay, then the “Hardware Not OK latch” circuit 3816 will latch. Selecting, e.g., pressing, the mode switch 3818 will unlatch the Hardware Not OK latch circuit 3816.

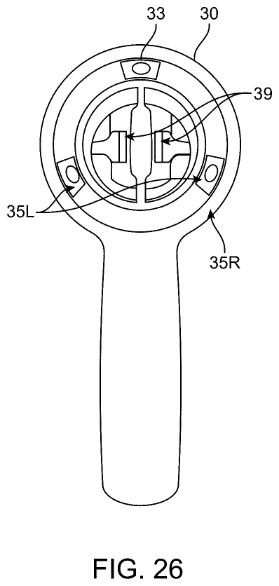

The microcontroller unit (MCU) 3802 also receives a few additional input signals from the rest of the console circuit board. When in the mode switch input to the MCU 3802 is a STANDBY mode, the TX signal indicates if both torsion springs 3903 are in contact with the blade 36 in the connected bulk tissue reducer 30. When in a READY mode, the TX signal is the measured source voltage for the blade voltage detection circuit 3906. The Activation Switch signal indicates if either activation switch is pressed. The Mode Switch signal indicates if the Standby/Ready switch 33 (FIG. 26) on the bulk tissue reducer 30 or the reset button 63 (FIG. 6) on the console 60 is pressed. The MCU 3802 uses the input signals to determine if system mode is in Standby mode or Ready mode and outputs the READY signal accordingly. The MCU3802 also uses all of these input signals to output the Software Driven Motor Control signal as a PWM signal to the logical AND gate 3804.

As shown in FIG. 38, the AND gate 3804 requires that the following conditions are met before activating the motor controller: the Software Driven Motor Control PWM duty cycle is greater than 0%, an activation switch is pressed, the hardware is OK, and the Hardware Not OK Latch circuit 3816 is not latched. If all conditions are met, the AND gate 3804 activates the motor controller 3806 and passes the PWM signal to it as the Hardware Gated Motor Control signal. The program code running on the MCU 3802 can provide redundancy for the hardware logic circuit by performing the same logic using the same inputs. If there is a circuit failure that prevents the hardware logic circuit from deactivating the motor 51, the MCU 3802 will still output a PWM signal with a 0% duty cycle. This signal will be passed on to the motor controller and still deactivate the motor 51.

FIG. 39 describes the high-level hardware schematic. The console 60 can include an AC-DC converter 3901 that converts AC Mains into a DC voltage, e.g., +24 VDC through a 60601-1 certified power supply or the like. It uses that initial DC voltage to step down to a voltage, e.g., +5 VDC to the handpiece attached detection circuit 3902 which measures the electrical current draw of the bulk tissue reducer 30 and uses that measurement to output the Handpiece Disconnected signal back to the console 60. The voltage provided to the handpiece attached detection circuit 3902 is also applied to the current limit circuit in the cutter handpiece.

The current limiting circuit 3910 in the handpiece 30 restricts the amount of current that can reach the blade 36. The electrical signal passes from the current limiting circuit 3910 to the Tx function check hardware 3911, which is comprised of two torsion springs 3903A, B (generally, 3903). The signal goes from one torsion spring 3903A, through the blade 36, and back through the second torsion spring 3903B. The signal that is received by the second torsion spring 3903B is returned to the console 60 as the TX signal. The handpiece 30 also contains three switches: one Standby/Ready switch 33 and two Activation switches 35R, L (see FIG. 26). The pushing of these switches modifies the signal returned to the switch hub circuit 3904 in the console 60, which determines which switch was pressed. If the standby/ready switch 33 is pressed, the signal returned to the switch hub 3904 is one voltage, e.g., close to 1.0 V, while the signal is another voltage, e.g., close to 3.0 V, when activation switch 35L, R is pressed.

The console 60 also can include an isolator circuit 3907, a container attached detection circuit 3908, and a blade voltage detection (w/filtering) circuit 3906 which receive electric signals from the container 40. The isolator 3907 provides an isolated voltage to the container attached detection circuit 3908 which transmits that to the container 40 through one of the conductive elements of the container cable 300 and the electrical connection comprised of the rivet 3611, spike washer 3612, and ring terminal 3613. An isolated voltage is provided to prevent interaction of the Container Attached Detection currents and the Blade Voltage Detection currents. If the container 40 is attached and intact, the isolated +5V voltage will travel through the conductive layer of the container 40, the second electrical connection, and the second conductive element back to the container attached detection circuit 3908. Based on the voltage it detects, the container attached detection circuit 3908 will output a signal to the isolator 3907, which converts it to the container disconnected signal.

If the blade 36 contacts the conductive layer of the container 40, the small difference between the TX signal and the RX signal is then filtered by the blade voltage detection (w/filtering) circuit 3906 and measured. Blade voltage filtering is a critical part of the hardware schematic. When the blade 36, or obturator, initially comes into contact with the tissue inside of an intact, or non-breached, container, the blade voltage detection (w/filtering) circuit 3906 detects a momentary change in voltage, which could be incorrectly interpreted as a signal signifying blade-to-container contact. The blade voltage filter can be a resistor-capacitor circuit (can be one or more resistor-capacitors) that decreases the amplitude and duration of this momentary voltage change to minimize the likelihood of a false triggering of the auto-shutoff feature. If the voltage measured by the blade voltage detection (w/filtering) circuit 3906 remains below a certain threshold, the Mesh Contact signal will signify that blade contact with the conductive layer of the container 40 has been made. The signals between the console 60, the bulk tissue receiver 30, and the container 40 can pass through components that limit the effect of electrostatic discharge (ESD) and protect the electrical circuit components from failing.

Referring now to FIG. 26, as previously mentioned, the bulk tissue reducer 30 can have three buttons. The button 33 in the 12 o'clock position toggles the device between ‘standby’ and ‘ready’ modes. The standby/ready toggle button 33 may have an LED that illuminates a first color (e.g., orange) when in a standby state during which the bulk tissue reducer 30 cannot be activated, and illuminates a second color (e.g., green) when in a ready mode the bulk tissue reducer 30 can be activated When the bulk tissue reducer is first plugged in, this button illuminates the first color, e.g., orange, which represents the standby mode, which means that the bulk tissue reducer 30 cannot be activated even if either of the trigger buttons is pressed. In order to take the bulk tissue reducer 30 out of standby mode press this button once and the light will turn the second color, e.g., green; this signifies the bulk tissue reducer 30 is ready for use. The buttons 35L, 35R at the 4 o'clock and 8 o'clock positions, respectively, activate the bulk tissue reducer 30. An operator may find either the left 35L or right 35R button more comfortable based on a hand position.

With further regard to the auto-shutoff feature, if the system 2500 is in a ready mode, or “armed”, the indicator 503 shown in FIG. 25 has turned green, the tissue cutting blade 36 will rotate upon activation of a trigger button(s) of the bulk tissue reducer 30, but will stop spinning if it any part of it comes in contact with the conductive mesh of the container 40. If the cutter blade 36 while rotating touches the steel cloth (see FIG. 10) in the container 40, the power to the bulk tissue reducer 30 is turned off and the blade 36 stops spinning. In some embodiments, the system 2500 may include a braking mechanism that can stop the blade 36 from spinning in a short amount of time, for example, 20-30 ms, or other time sufficient to prevent the blade from spinning after it makes contact with the conductive layer, and to prevent it from damaging the conductive layer(s) between the conductive layer and the interior of the container 40. When properly placed, the bulk tissue reducer 30 should pass within the container 40 through the vagina or abdominal incision and cut the specimen within the container at a location in the abdominopelvic cavity. At this point, the user will get alerted of the stop (or auto-shutoff) and the system will immediately shift into standby mode preventing the blade from moving and turning the indicator light orange or yellow. The user can hit the standby/ready toggle button 33 on the bulk tissue reducer 30 or the button 503 on the console 60 and continue the procedure or determine other courses of action, for example, with respect to cylindrical spinning blade that performs the cutting of the specimen 15.

The bulk tissue reducer 30 can be deployed together with an obturator 31, for example, as shown and described in other embodiments herein, in particular, FIGS. 28A-32. The obturator 31 ensures that the blade 36 of the bulk tissue reducer 30 slides smoothly into the container 40. To remove the obturator 31, a user may depress its two release tabs 39 and pull gently. When replacing the obturator 31, the user should ensure that the plastic rib is in the vertical (or 12 o'clock/6 o'clock) position and both release tabs 39 (on the 3 o'clock and 9 o'clock positions) click into place.

In some embodiments, the oversized tenaculum 45 may be used as is typical for grasping and manipulation of a tissue specimen 15. This component 45 may also be used to grasp tissue 15 and pull it through the spinning cylindrical blade 34 of the bulk tissue reducer 30. In some embodiments, as shown in FIG. 25A, the tenaculum 45 can have a small outer diameter, e.g., 5 mm diameter, rather than a larger (e.g., 10 mm) diameter. For example, an outer diameter (D2) of the tenaculum 45 may be five times smaller than the inner bore diameter (D1) of the cutter of the bulk tissue reducer 30. In some embodiments, the ratio of diameters D1/D2 may be about 1.5 to about 10, more specifically, about 5 to about 8.

Another reusable component is the console 60, which may include the optional drive box 50, and therefore include similar components, the descriptions not repeated for brevity. In some embodiments, the console 60 has a main power switch which can be in the back which needs to be activated. On the front panel, as previously described, there are three features of importance. First, the bulk tissue reducer (BTR) drive cable is plugged into a first connector 61, which can be keyed such that it can only be attached in one orientation with the black arrows on the connector in the 12 o'clock position. When the BTR connector is properly seated, the light indicator 501 around the connector 61 will turn green. When the container connector 300 is properly seated in the second port 62, the light 502 around it will also turn green. When both the bulk tissue reducer connector and container connector have green lights, the system can be toggled between standby and ready mode by pressing the “Standby/Ready” mode button 63.

For some method embodiments, a pre-procedure system setup embodiment that includes the system 2500, as shown in FIG. 25, in accordance with embodiments described above may include the following steps. First, determine if the patient is a proper candidate for the system 2500. A user should verify that the patient's vagina 24 can accommodate the outer diameter or transverse dimension of the bulk tissue reducer 30 which may, in some cases, have an outer diameter of up to about 42 mm, or any other outer diameter suitable for the particular procedure being performed. Next, ensure that standard laparoscopic tools 140 needed for the procedure are available, including but not limited to: a laparoscope, atraumatic graspers 45, 106, sterile surgical lubricant, and/or other instruments required for surgery as specified by the surgeon. Ensure that the non-sterile console 60 is plugged into the wall outlet and is positioned about 1.3 meters or 4 feet away from the patient's vagina 24. Position a stand at the perineal field which can hold the bulk tissue reducer 30, container 40, tenaculum 45, and vaginal occluder of choice as needed. In general, the patient 20 is to be prepared per the institution's standard of care for laparoscopic hysterectomy procedures. Use of the system 2500 may begin once the colpotomy is complete and the uterine manipulator has been removed in some instances. For some cases, ensure the patient is in Trendelenburg position and occlude the vagina to maintain pneumoperitoneum.

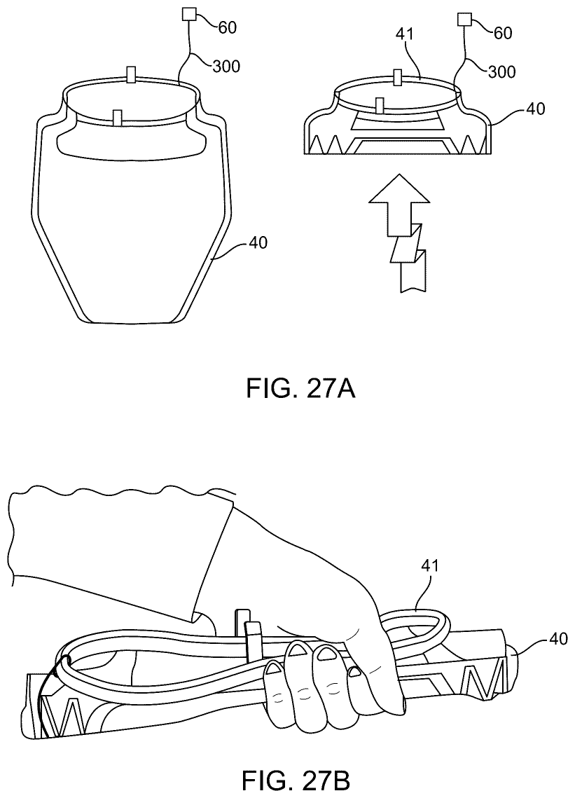

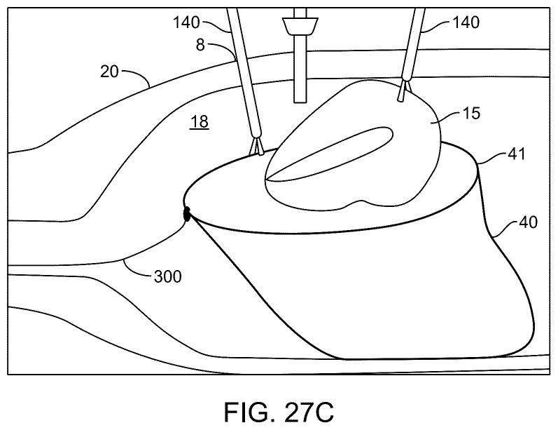

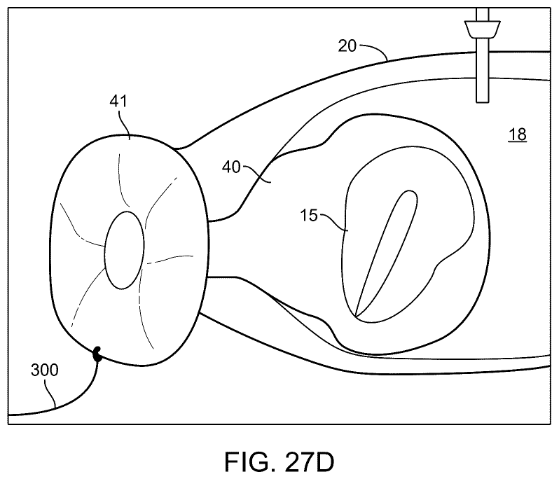

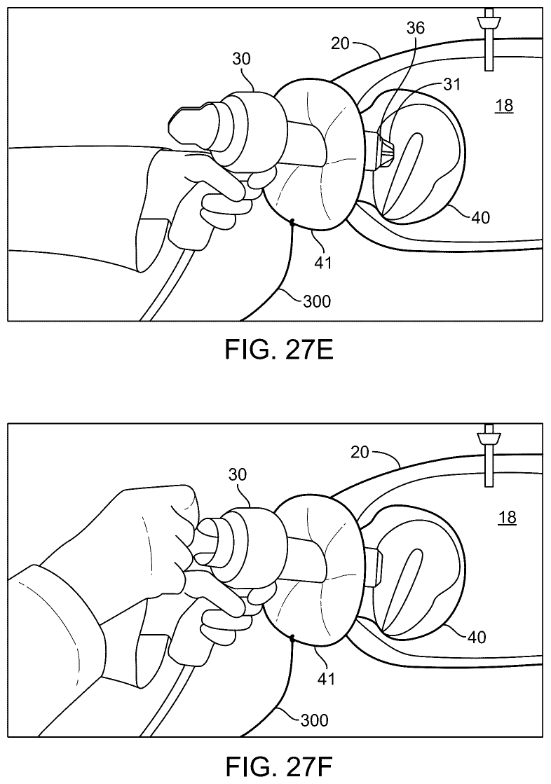

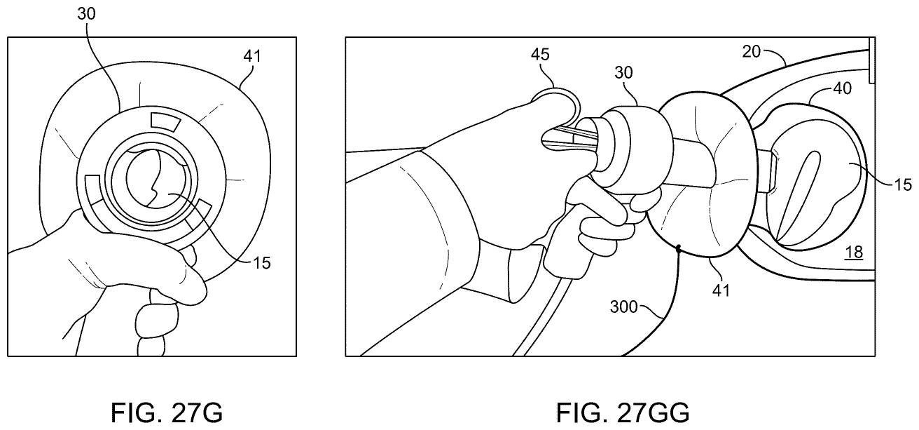

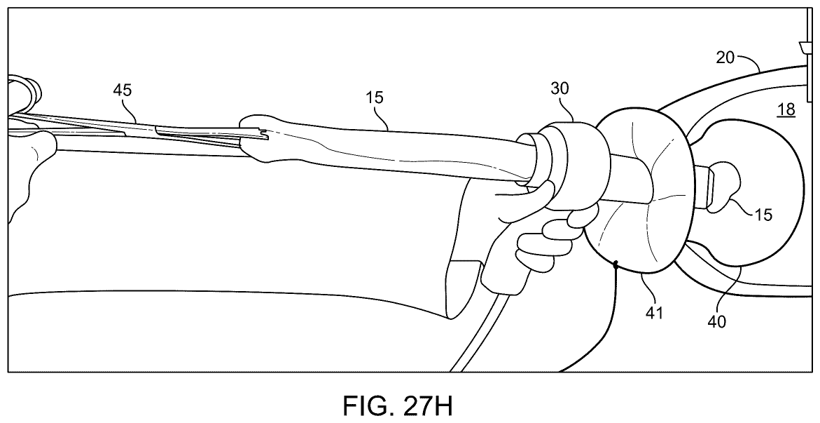

Some procedure embodiments may include inserting the tissue container 40 through the vagina 24 and into the abdominal cavity 18 and applying generous amounts of sterile lubricant on the inner surface of the container 40. In some embodiments, the tissue container 40 may be pre-lubricated with lubrication already placed into the sterile bag. After lubrication, the user may fold the tissue container 40 in an accordion shape with each fold being about 4 cm wide as shown in FIG. 27B. The user can remove the vaginal occluder and insert the tissue container 40 into the abdominal cavity 18 through the vagina 24, being careful to avoid fluid splatter. The user should ensure that the opening of the tissue container 40 faces up and that the tether side is the last part inserted. The user should not insert the tether connector 300 into the body 20.

In some cases, the user may change gloves and gown per institutional practice and move from the perineal field to abdominal field. The user can roll the uterus or other tissue 15 into the interior volume 42 of the tissue container 40 using atraumatic graspers 140 through the abdominal ports 8 (see FIG. 27C). The user can grasp the two yellow tabs 3616 using the two atraumatic graspers 140 and gently shake as needed to ensure that the specimen 15 is sufficiently deep in the interior volume 42 of the tissue container 40. The user may then pull the tether 300 to start extracting the rim 41 of the tissue container 40. Once the leading edge 41 of the tissue container 40 is at the opening of the vagina 24, the user may use fingers to extract the entire rim 41 of the tissue container 40. The user can roll the rim 41 of the tissue container 40 as needed to ensure the specimen 15 is near the vaginal cuff, as shown in FIG. 27D. The user may then hand the container cable 300 to a non-sterile person to plug it into the console 60. The user should verify that the green light 501 on this port 61 is displayed once the tissue container 40 has been plugged in.

The user may then hand the drive cable 56, 3215 to a non-sterile person to plug into the console 60. Once plugged in correctly, the cable port indicator will display a green light. The user can confirm that the obturator 31 is locked into place by gently pulling back on the obturator. The user can apply a sterile lubricant onto an outside surface of the cannula 3201 of the bulk tissue reducer 30. The user can insert the bulk tissue reducer 30 with the obturator 31 into the tissue container 40 and vagina 24 until the obturator 31 is in firm contact with the tissue specimen 15 within the abdomen 18 as shown in FIG. 27E. The user may then remove the obturator by squeezing the two tabs 39 at the rear of the obturator 31 and then pulling it out as shown in FIG. 27F. The user can visualize the tissue 15 that needs to be reduced by viewing it down the bore of the bulk tissue reducer 30 as shown in FIG. 27G. If the tissue 15 cannot be visualized, the user can tighten the tissue container 40 by rolling the hoop 41 and applying proximal retraction of the tissue container 40 if necessary. At this point, the user should be able to visualize the tissue 15 up against the distal end of the bulk tissue reducer 30.

In some instances, it may be useful for the user to ensure that the tissue specimen 15 is disposed against the cutter 36 and that none of the container wall 44 is between the cutter 36 and the tissue specimen 15. The user can pass the tenaculum 45 down the bore of the bulk tissue reducer 30 and grasp the uterus 15 as shown in FIGS. 27F and 27G and pull it towards the cutter 36. The user should put the system 2500 in ready mode by pressing the “Standby/Ready” button 33 once. The user should activate the bulk tissue reducer 30 using the hand switch 35L, 35R and extract portions of the uterus 15. With the blade 36 spinning, the user can push the bulk tissue reducer 30 into the tissue specimen 15 while simultaneously pulling on the tissue specimen 15 with the tenaculum 45 with the other hand. To stop cutting, the user can release the button 35L, 35R. The user can reposition the bulk tissue reducer 30 as necessary and repeat cutting steps until the tissue container 40 and any remaining specimen 15 is small enough to be delivered through the vagina 24. If the bulk tissue reducer 30 is removed from the vagina 24 during repositioning, the user should ensure that the obturator 31 is locked into the bulk tissue reducer 30 before re-inserting it into the body 20. It may be helpful to further roll the container rim 41 between cuts as the remaining specimen 15 shrinks in size.

In some embodiments, it may be possible that during tissue extraction the console 60 will make an audible tone and the cutting will stop. This indicates that the “auto-shutoff” feature described herein has triggered because the blade 36 made undesirable contact with the conductive layer 100 of the tissue container 40. The user should adjust the position of the bulk tissue reducer 30 so that it is not making contact with the tissue container 40 and put the system 2500 back into “Ready Mode” to proceed. When the tissue specimen 15 has been reduced substantially and cutting is complete, the user may place the system 2500 into a standby mode by pressing the “Standby/Ready” button 33 and remove the bulk tissue reducer 30 from the vagina 24. Keep the bulk tissue reducer 30 within the surgical field in case it is needed again. The user should pull gently on the tissue container rim 41 to remove the tissue container 40 (and any tissue remaining within) by extracting it through the vagina 24 as shown in FIG. 27I. Again, care should be taken to minimize bodily fluid spatter. The user should then re-occlude the vagina 24. If the container 40 and specimen 15 cannot be pulled through, The user should repeat extraction steps as necessary.

In some cases, for the bulk tissue reducer 30 to work effectively, the blade tip 36 should be beyond the vaginal cuff and in the abdominal cavity 18, rather than inside the vaginal canal 24. Cutting may best be accomplished in some instances by a combination of pushing the cutter 30 all the way into the tissue container 40 while pulling on the tenaculum 45. Care may be taken to maintain enough forward pressure on the bulk tissue reducer 30 so that the cutting edge 36 does not back up into the vaginal canal 24 where it may be less effective at cutting.

It may be undesirable in some cases to pull on the tenaculum 45 with excessive force as this may result in the instrument pulling off of the specimen 15. Because the bulk tissue reducer 30 may be large, harder or calcified tissue 15 may be cut relatively slowly—in this scenario the system 2500 may work best when pushing/pulling with moderate force and allowing for a longer duration cut rather than pulling harder on the tenaculum 45. If the console 60 makes an audible tone and the cutting stops, this generally indicates the auto-shutoff feature has triggered because the blade 34 made contact with the conductive layer of the tissue container 40. In response, a user of the system 2500 may adjust the position of the bulk tissue reducer 30 so that it is not making contact with the container and put the system back into “Ready Mode” to proceed.

Note that if the system 2500 senses a high cutting resistance, it may reduce the blade speed to maintain smooth operation. It may be desirable for a user of the system 2500 to maintain steady force on the tenaculum such that the bulk tissue reducer will continue to cut. This may be part of a normal functioning of the device. If the device has cored into the specimen 15 but cannot go deeper and it is physically hard to extract tissue 15 through the bulk tissue reducer 30, a user of the system 2500 may try to remove the bulk tissue reducer 30, rotate the specimen slightly, and approach the specimen 15 at a different location or angle. If the blade 34 does not spin, a user of the system should confirm that the system 2500 is powered on, in ready mode, and that all 3 indicator lights 501, 502, 503 on the console 60 are green.

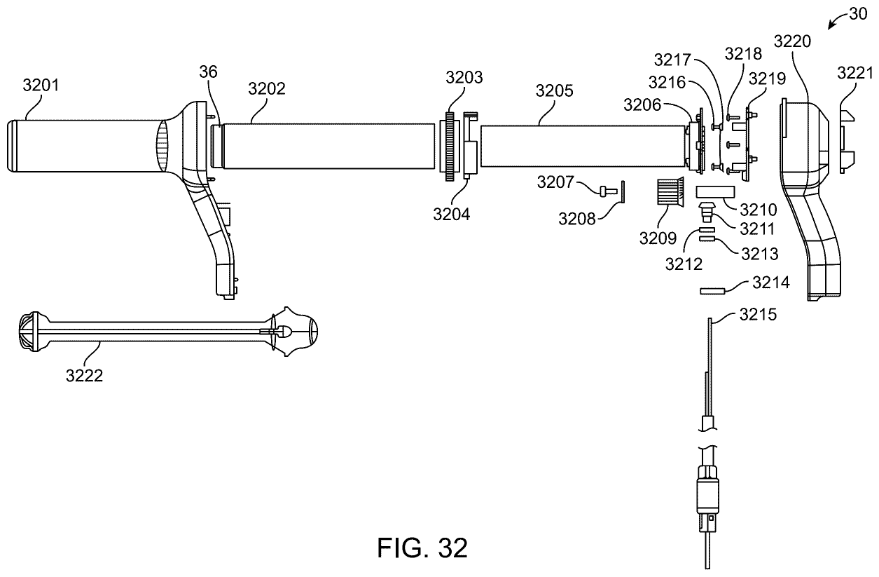

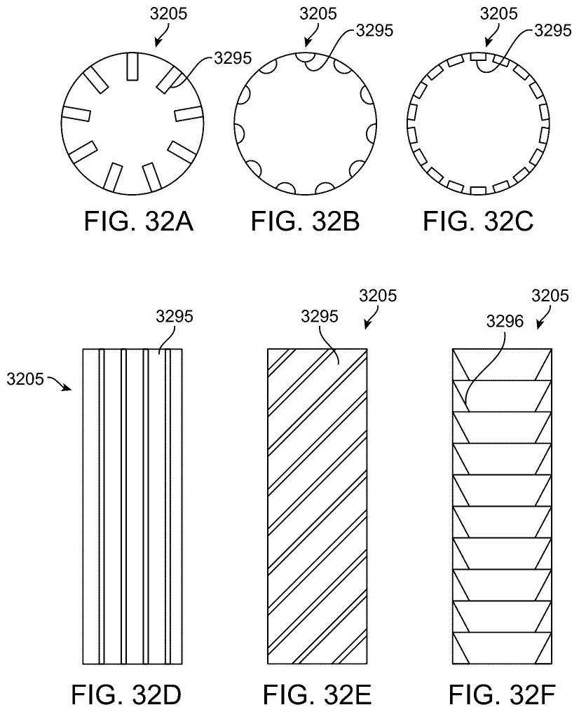

An orange or yellow light around a connector may indicate that either the system 2500 is not plugged into that connector, or not functioning correctly. In such circumstances, a user of the system may remove the cable from that connector and reinsert it. FIG. 32 is a schematic of an embodiment of a bulk tissue reducer 30 of FIGS. 28-31. As shown, the bulk tissue reducer 30 may include a distal shell with a cannula 3201, a blade 3202, a blade coupler 3203, a blade coupler bearing 3204, an anti-rotation tube 3205, a handpiece board cap 3206, a socket head thread locking screw 3207, an oversized washer 3208, a spur and bevel gear 3209, a threaded standoff 3210, a flexible shaft adapter 3211, a first shaft ball bearing 3212 (e.g., 5/16″), a second shaft ball bearing 3213 (e.g., ¼″), a hose clamp 3214, an handpiece umbilical assembly 3215, a thread-locking screw 3216 (e.g., 6 mm), a torsion spring 3217, thread forming screws 3218, a handpiece circuit board 3219 (e.g., PCBA), a proximal shell 3220, a button gasket 3221, and an obturator 3222. The distal shell with a cannula 3201 acts as half of the enclosure that contains the rest of the bulk tissue reducer 30 components.

The long cannula 3201 provides a low-friction atraumatic surface that facilitates insertion of the bulk tissue reducer 30 into the patient 20. The blade 3202 provides the sharpened edge that is used to cut the tissue. The blade coupler 3203 is a spur gear that is bonded to the blade 3202. It transmits the rotational motion from the spur and bevel gear 3209 to the blade 3202. It also changes the torque and speed output of the drivetrain based on its gear ratio (e.g., 3:1) relative to the spur and bevel gear 3209. The blade coupler bearing 3204 guides and facilitates the rotational motion of the blade coupler 3203 to minimize vibration and noise when the bulk tissue reducer 30 is activated.

The anti-rotation tube 3205 is configured to prevent the tissue 15 from spinning as it is pulled through the bore of the bulk tissue reducer 30. Additionally, it transmits light from the LEDs on the cutter handpiece shell 3220 down to and out of the distal end of the blade 3202. This light illuminates the target tissue and allows the user to visualize the working area prior to activating the bulk tissue reducer 30. The handpiece board cap 3206 secures the position of the AR tube. It also compresses the button gasket 3221 to create a seal that protects the handpiece 30 from shorting due to fluid ingress. The socket head thread locking screw 3207 screws into the threaded standoff 3210 and holds the oversized washer 3208 in place.

The oversized washer 3208 supports the spur and bevel gear 3209 and keeps it in mesh with the flexible shaft adapter 3211. The spur and bevel gear 3209 is responsible for transmitting the rotational motion between the flexible shaft adapter 3211 and the blade coupler 3203, which have perpendicular rotational axes. It also changes the torque and speed output of the drivetrain based on its gear ratio (e.g., 2:1) relative to the flexible shaft adapter 3211. The threaded standoff 3210 acts as a gear shaft for the spur and bevel gear 3209 and helps the socket head thread locking screw 3207 hold the oversized washer 3208 in position. The flexible shaft adapter 3211 transmits the rotational motion from the flexible shaft in the handpiece umbilical assembly 3215 to the spur and bevel gear 3209. The first shaft ball bearing 3212 and the second shaft ball bearing 3213 facilitate the motion of the flexible shaft adapter 3211 to minimize vibration and noise when the bulk tissue reducer 30 is activated. The hose clamp 3214 secures onto the handpiece umbilical assembly 3215 and acts as a strain relief that constrains the motion of the handpiece umbilical assembly 3215 relative to the bulk tissue reducer 30 and prevents it from stressing its electrical wires.

The handpiece umbilical assembly 3215 provides an electrical connection from the bulk tissue reducer 30 to the console 60. It also contains a flexible shaft that couples the rotational motion from the Console motor to the flexible shaft adapter 3211. The thread-locking screw 3216 secures the torsion spring 3217 against the metal standoff of the handpiece PCBA 3219. The torsion spring 3217 transmits the electric auto-shutoff signal from the metal standoff of the handpiece PCBA 3219 to the blade 3202. In the bulk tissue reducer 30, there are two torsion springs 3217 that each have two arms. The spring force of the torsion springs 3217 acts to push the arms in contact with the blade 3202. This provides four points of electrical contact with the rapidly rotating blade 3202 to which the auto-shutoff signal can be conducted from the handpiece PCBA 3219. The thread-forming screws 3218 screw into the proximal shell 3220 and holds the handpiece board cap into position. The handpiece PCBA 3219 contains LEDs to illuminate the target tissue, orange/green LEDs that inform the user of the system mode, momentary switches that allow the user to activate the bulk tissue reducer 30 or change the system mode, and an electrical circuit that transmits the auto-shutoff signal through its metal standoffs.

The proximal shell 3220 acts as half of the enclosure that contains the rest of the bulk tissue reducer 30 components. The button gasket 3221 creates a seal that prevents fluid ingress into the bulk tissue reducer 30. The obturator 3222 is a removable part that, when inserted into the bulk tissue reducer 30, prevents the blade 3202 from cutting. For example, the distal shell 3201 coupled to the proximal shell 3220 form a handpiece. The tubular cutting blade assembly 3202 can rotate inside the cannula 3201 by the coupler 3203, which may include gears or the like inserted inside the proximal shell 3220. The distal end of the tubular cutting blade assembly 3202 includes the actual blade 26, which rotates and in doing so cuts the specimen 15 so that it is of a sufficient dimension for insertion into the lumen of the blade tube 3202.

However, due to the spinning tubular cutting blade assembly 3202, the portion of the tissue specimen inside the lumen likewise rotates which causes difficulty during a procedure when attempting to remove the specimen from the proximal end of the tubular cutting blade assembly 3202. The anti-rotation tube 3205 resolves this problem by remaining stationary inside the lumen of the tubular cutting blade assembly 3202 while the cutting blade assembly 3202 is spinning. The anti-rotation tube 3205 is configured for insertion through a lumen of the cutter. The main gear is attached to the outside of the blade which allows for the anti-rotation tube 3205 to fully cover the blade and guide the extracted tissue towards the exit of the device of a proximal end thereof in a smooth and low friction fashion. Other gears 3231,3232, 3233 can contribute to the rotation of the blade. Accordingly, the specimen after being cut by the blade 36 moves through the anti-rotation tube 3205 and therefore does not exhibit an undesirable rotation as it is being removed from the proximal end.

It may be desirable to introduce friction between the specimen inside the anti-rotation tube 3205 and the lumen wall of the anti-rotation tube 3205 in some cases. When the tubular cutting blade assembly 3202 spins, it can cause the specimen to rotate inside the anti-rotation tube 3205 even though the anti-rotation tube 3205 is stationary.

To reduce or eliminate such rotation, FIGS. 32A-32F show various schematic embodiments of the anti-rotation tube 3205, which is fixed relative to the cutting blade assembly 3202, which has a blade 3224 that rotates inside a housing of the assembly 3202. The anti-rotation tube 3205 can include a high friction interior or lumen surface configuration that includes longitudinal flutes or ridges 3295 in a spiral configuration, a straight or extruded configuration which can look like teeth, or fins, or the like as otherwise shown in FIGS. 32A-32F. FIG. 32F shows radial ridges 3296 that present themselves down the inner lumen of the anti-rotation tube. These ridges can be configured into cross sections like triangular teeth which make it easy to pull tissue in a proximally retracting direction, but not in the distally advancing direction. This pattern or finish on the inside of the anti-rotation tube can be presented as a small/low slope angle of a triangle then leading to a sharp angle. It may be easier to pull tissue along the low slope angle, but harder to pull it against the harder slope angle in some instances.

Referring again to FIGS. 33-36, one or more rim tabs 3616 or rim hoops can be coupled to the tubes 3604 with respect to the cable 72 to assist with manipulation of the container 40. For example, as described herein, atraumatic graspers may be used to grab the tabs 3616 or grasp through the tabs or loops to help control the rim 41 when loading a specimen, shaking the container to ensure that the specimen slides to the bottom, or at any other suitable time. The tabs 3616 may also be configured such that two atraumatic graspers can fit into one tab and such that one grasper can hand off a second loop to a second grasper. Tab or loop embodiments on the multilayer container, for example, illustrated in FIG. 36, may be used to assist with manipulation of the container in some cases. Atraumatic graspers 140 or other instruments may be used to grab the tabs 3616 to help control the rim of the container when loading the specimen, shaking the container to ensure that the specimen slides to the bottom, or at any other suitable time. Tab embodiments may also be configured such that two atraumatic graspers can fit into one tab and such that one grasper can hand off a second loop to a second grasper.

In some embodiments, as shown in FIG. 36, two elongated portions 3602 of the container 40 may extend from a main body of the container 40. The elongated portions 3602 may be formed in a same manner as a main body, for example, a laminated mesh 3610 shown in FIG. 36A, or other embodiments of the container 40, for example, described herein. The rim 41 can be coupled to the elongated portions 3602, for example, described in FIGS. 33 and 33A, for example, using the rivet or eyelet 3611, spike washer 3612, and ring terminal 3613 to provide an electrically conductive connection between the rim 41 and conductive elements extending through the elongated portions 3602 and the container body. The rim 41 may further include one or more tubes, tube ribbons, or other elongated elements 3604 formed of polyurethane or other polymer and/or natural material. The tubes 3604 may be coupled to the elongated container portions 3602 by the combination of eyelet 3611, spike washer 3612, and ring terminal 3613, and/or other coupling mechanisms. Other components may include a PTFE beading 3607, and conductive element 3608 such as a cable, tinsel wire, etc. which is configured to be in direct contact with container cable card 3617. In some embodiments, the conductive element 3608 may be the cable 300 described in FIGS. 11-11C. With further reference to FIG. 36C, two halves of the container body are shown which when folded and sealed at the laminated edges 3620A, 3620B (generally, 3620) can form the container, for example, shown in FIG. 36D, having an interior volume.

The eyelet 3611 or grommet or the like can operate as a rivet that secures the spike washer 3612 and ring terminal 3613 in position. It also contacts the conductive layer of the container 40 and conducts electrical signals from the container 40 to the ring terminal 3613. The spike washer 3612 penetrates the conductive layer of the container 40 and conducts electrical signals from the container 40 to the ring terminal 3613. The ring terminal 3613 secures the conductive element 3608 and conducts electrical signals from the eyelet 3611 and the spike washer 3612 to the conductive element 3608. The conductive element 3608 conducts electrical signals from the ring terminal 3613 to the console 60 The conductive element 3608 (for example, which may be part of the cable 300) is wrapped around the container cable card 3617 which secures the conductive element 3608 and helps the user organize it during use. The rim 41 provides resilient structure to the opening of the container 40 which allows it to spring open after insertion into the abdominal cavity to assist with containment of a tissue specimen. The rim 41 can also be rolled to shorten the effective length of the container 40 which helps tissue extraction with the bulk tissue reducer 30. The PTFE beading 3607 helps align the ends of the tubes 3604 to form the rim 41. The rim tab 3616 provides an area that can be grabbed with atraumatic graspers to manipulate the rim 41 during containment.

In fabricating some tissue container embodiments 40, for a composite layer embodiment of some tissue container embodiments 40, the seam seal 1722, 1724 (for example, shown in FIG. 17I) may be approximately 0.276 inches outside and 0.276 inches inside of the cut mesh. These inside and outside dimensions may range in some cases from about 0.05 inches to about 1 inch. Seam seal embodiments 1722, 1724 may be optimized to provide a robust seal that resists being torn or penetrated. Additionally, the seam seal 1722, 1724 may be configured to remain flexible so that it can be inserted into the body 20 and manipulated during the containment and extraction process. The tubing insert embodiments, shown as PTFE beading 3607, may be used to provide alignment to the Polyurethane ribbon 3604 during manufacturing or at any other suitable time. Some rim tubing embodiments 3604 may be made of 3 permanently bonded plastic tubes (such as TPU). The material of the rim tubing 3604 may have a durometer of about 90D with a potential range from about 30 shore D to about 95 shore D and may be configured to be flexible enough to be compressed during insertion of the container through the vagina, but stiff enough to spring open in the abdominal cavity during specimen containment. The tubes and durometer of the rim 41 may be configured to allow a user to shorten the length of the tissue container 40 by rolling the rim without any aid in some instances. The rim 41 may also have a stainless-steel or nitinol ribbon or core which may be configured to provide flexibility and resilience to spring into a pre-determined shape like a circle or an oval to facilitate opening the tissue container 40 to load tissue 15 into it. In some embodiments, the tissue container 40 does not have a rim 41, but rather an edge that forms the perimeter about the opening to the interior 42 of the tissue container 40.

As described above, some tissue containment and removal system embodiments 10 or components thereof may include additional contact detection system embodiments having any of the suitable features. dimensions. or materials as those discussed above as well as other features. In particular, as described in FIGS. 3 and 25. an auto-shutoff system can be provided to prevent injury or damage to the tissue container 40 in the event that a surgical tool inside the tissue container 40, in particular, the bulk tissue reducer 30 approaches. is proximity to, or directly contacts a wall 44 of the tissue container 40. Some such additional system embodiments 10 may include contact detection system embodiments as shown in the schematic representations of FIGS. 37-39. Some such system embodiments, in particular, those with auto-shutoff feature, may be designed such that if any of the electrical connections are broken during a procedure wherein a surgical tool is positioned in the tissue container 40. a surgical tool. e.g . . . the bulk tissue reducer 30 or the like. cannot be activated. The following are some of the scenarios that may be configured prevent compromise of the integrity of the wall 44. For some auto-shutoff system embodiments. if the conductor 72 to the tissue container 40 is open or broken it presents a higher resistance (for example greater than 1 k ohm) then the motor 51 of the bulk tissue reducer 30 cannot be activated. If voltage or ground lines going to the handpiece of the bulk tissue reducer 30 are disconnected. the bulk tissue reducer 30 does not draw enough current. and thus the motor 51 is not activated. If the switch connection is broken, the motor 51 also cannot be activated. If the function check signal is broken a software check will fail and thus the motor 51 cannot be activated. A final check of the presence of the tissue container 40. quality of the tissue container 40, and authenticity of the tissue container may be performed before the motor 51 may be activated or otherwise enabled.

Embodiments of the tissue containment and removal system 10, 2500 may also be configured such that auto-shutoff system embodiments therein perform the same regardless of whether the tissue container 40 is filled with air, blood, tissue, other bodily fluids or a mixture thereof. This may be achieved in some instances by using a circuit of resistor-capacitor (RC) filters (not shown). Such a circuit may be tuned to only allow auto-shutoff signal frequencies that behave similarly regardless of the tissue container contents. Such an RC circuit may also be configured to minimize false auto-shutoff triggering. This may all be accomplished for some embodiments by modifying the resistor and capacitor values of each filter, as well as the number or type of RC filters in the circuit. For some embodiments, this RC circuit may include 2 or more low-pass RC filters in-line with the auto-shutoff signal using a combination of electrical circuit components, such as resistors, capacitors, and so on.

If the tenaculum 45 is in contact with the conductive layer 100 of the tissue container 40, the auto-shutoff may be triggered if the tenaculum 45 is wired in electrical communication to the console 60, the cutting handpiece 30, or an intermediate connection. Additionally, in the event that a tenaculum or grasper 45 contacts the conductive layer 100 the system may be configured to prevent an auto-shutoff trigger through insulation, grounding, or material choice, etc.

Some software control function embodiments may be configured such that when the hardware signals that the tissue cutting blade 34 should be spinning, the software ramps up the motor Pulse With Modulation (PWM) to 100%. All hardware checks shown in FIG. 37 must pass before the tissue cutting blade 36 can spin (i.e. if the software failed and always output PWM of 100% the function of the system would be identical, but the handpiece 30 would jerk a bit due to faster blade acceleration). When the hardware signals that the tissue cutting blade 36 should stop spinning (under normal non-mesh contact conditions) the software ramps down the PWM to 0%.

The ramp down occurs over ˜150 ms, and reduces jerking of the handpiece 30 in the user's hand. Regardless of whether the software stops the motor 51, the hardware stops the motor after 250 ms. The software may also be configured to reduce the blade's speed of rotation if it senses or otherwise detects a high torque load on the drivetrain. In some cases, the hardware may be configured to stop the blade 34, 36 entirely if a higher torque limit is reached. The software may also be configured to control the color of some indicators 501, 502, 503, which may include Light Emitting Diodes (LEDs), of the system.

Some control software of the system may include functions for safety checks such that the software may be configured to double check that the hardware is ok before spinning the tissue cutting blade 34, 36. The software may also be configured to brake the motor 51 if it sees or otherwise detects a problem with the hardware (as a double check on the hardware). The software may be configured to output an audible tone if there is tissue cutting blade 36 to tissue container 40 contact made. In an unlikely case of software failure, the system may be configured such that the tissue cutting blade 36 would not spin. In addition, the software may be configured to record and stream data for device diagnostic and usage monitoring purposes.

FIGS. 38 and 39 show a control schematic and block diagram, respectively, of a tissue containment and removal system embodiment 10, 2500. For some embodiments, as shown in FIG. 38, all inputs must be high to have a high on the output. The software driven motor control signal for such embodiments cannot pass unless the system is in READY mode, the user has activated the switch, the hardware is OK (see OR Gate), and there is no latched signal from the hardware not being OK. For some embodiments, any input going high will result in a high signal at the output. If contact between the conductive layer 100 and the cutter 36 is detected, the handpiece 30 or tissue container 40 is disconnected, the motor 51 draws too much current, or the system is in a reset condition e.g. if there is a loss of power the result will be that the hardware is not OK and the software driven motor control signal cannot pass.

In some embodiments, if a signal input, e.g., contact by an instrument 140 with the tissue container 40, motor overcurrent, etc. is received that indicates a problem or undesirable event with the system, then it can be triggered by a latch. The output will prevent the software driven motor signal from passing until the latch is cleared, for example, by moving the console 60 into a Ready Mode. As shown in FIG. 38, a motor controller 3806 accepts Motor Control Signal and for gradual increases or decreases in rotational speed, steady on, or braking where the terminals of the motor 51 are shorted to ground. The motor 51 may include a direct current motor and may be coupled to the handpiece blade 34 through a driveshaft 56.

In general, embodiments of the system start in “Standby Mode” when powered on (indicated by an orange light around the Standby/Ready button 33). The system may be put into ready mode (indicated by a green light) with a button push if there are no hardware issues in some cases. The blade/motor 34, 51 can only be activated in Ready Mode. Any hardware issue will return the system to Standby mode. As shown in FIG. 39, a voltage source, e.g. +5 Vdc is used for tissue container mesh (conductive layer 100) detection. Embodiments include a cutting device, such as tissue cutter 34, used for surgery that is configured to conduct an electrical signal. Redundant electrical contacts ensure the +5 Vdc can pass to the tissue container conductive element mesh 100 if contacted. The conductive element mesh 100 of the tissue container 40 with connection points to wires at two different locations on the tissue container 40. Both connection points must be robust so that less than 1kohm is presented Container Attached Detection.

Isolated power and signal allow the container Attached Detection and Blade Voltage Detection circuits to not interact such that they can share two conductors to the container. A DC voltage signal is connected to the blade. Electrical line monitored by the console, looking for blade contact to the container. Under Normal conditions, no current (<10 uA) from this circuit should flow to the patient. The current limit of 45 uA meets the Single Fault Condition limit, for example, in compliance with IEC 60601-1.

During operation, the user can press either the Activation Switch 35L, 35R, or the Mode Switch on the Handpiece 30. The switch signal from the Handpiece is converted to the signals indicating whether it was the Activation Switch or the Mode Switch. Current passing through the +5 Vdc source to the handpiece 30 indicates that the handpiece 30 is attached. Blade Voltage Detection is provided to detect that the +5 Vdc from the blade 34 has made contact with the mesh 100 in the tissue container 40. The circuit triggers if less than a predetermined resistance, is detected between TX and RX. Container Attached Detection Detects if there is less than 1kohm between the connection points to the container mesh 100. Tx Function Check When the system is not in READY the Software checks the continuity of the electrical connections to the blade 34 and doesn't allow the system to go into READY unless both connections are good.

Some aspects of embodiments of the tissue container 40 may include various material composites, which may be categorized as follows: multilayer including a stronger or stiffer layer that is coated, multilayer—including a strong or stiff layer which is laminated (with films on one or multiple sides), multilayer including weak or flexible layer in the middle that is conductive which can either be coated or laminated over, monolayer—weak or flexible layer that is conductive, and mono or multilayer having one or more colors or lubricated coatings. For some embodiments, as discussed in PCT/US2017/029162, which is incorporated by reference above, the tissue container 40 may include an inner bag constructed of high tensile-strength strips of metallic or plastic material embedded in or attached to a plastic or metallic material. In some embodiments, as described in PCT/US20/46135, which is incorporated by reference herein in its entirety, with respect to FIGS. 8 and 9, conductive ink including such materials as silver, carbon and the like may be used to create the conducive layer 47 or conductive element 46 of the tissue container 40.

In another embodiment, as also described in PCT/US20/46135 incorporated by reference above, conductive elements 46 may include a metal mesh layer, a conductive plastic, such as PEDOT or other similar material, conductive plastic mesh or plastic layer in the container construction. For some tissue container embodiments 40, the conductive layer 47 may include a woven mesh 100 as seen in FIG. 10 with a composite mesh structure that includes both strands of conductive material and strands of non-conductive material, described above. The mesh 100 may have one or more gaps ranging from 8-20 mm, but not limited thereto.

In some embodiments, the fabric of the tissue container 40 may include a Thermoplastic Polyurethane (TPU) coated fabric, e.g., formed of Kevlar®, and conductive stainless steel strands. In some embodiments, as shown in FIG. 40A, a material composite forming a multilayer tissue container 40 may include a coated layer 4002 disposed about a strong, puncture-resistant conductive layer 4004. 4014, and 4024 represent the electrically conductive materials such as metal mesh, coated fabric, conductive ink, conductive rubber, conductive polymers, conductive cotton, copper, gold, bronze, aluminum, silver, graphite or other materials. Coated layers 4002, 4012, 4022, and 4032 may include the insulative layers which include TPU, urethane, parylene, mylar (PET), natural rubber, polyethylene, polyester, polypropylene, Tedlar (PVF), Nomex®, Kapton®, Epoxy powder coating, etc. In some embodiments, a TPU or insulative stack may have a thickness of about 0.000001″ to about 0.004″, which may include a mesh and TPU stack material. The insulative material may be molded or extruded.

In some embodiments, as shown in FIG. 40B, a material composite forming a bilayer tissue container 40 may include an outer layer 4012 forming a film or coating over an outer surface of a strong, puncture-resistant conductive layer 4014. An inner surface of the conductive layer 4014 is not coated. In some cases, as shown in FIG. 40C, a material composite forming a multilayer tissue container 40 may include a strong, puncture-resistant conductive layer 4024 that is coated with a laminated film 4022. In some embodiments, as shown in FIG. 40D, a material composite forming a multilayer tissue container 40 may include a flexible conductive layer 4034 that is coated with a laminated film 4032. In some embodiments, the inner layer 4034 may be a flexible mesh, for example, formed of Sateen or related fabric weave structure In some embodiments, as shown in FIG. 40E, a monolayer conductive material composite 4042 may be used for forming a tissue container 40 such as conductive plastic, conductive rubber, or conductive film. In some embodiments, the inner layer may be formed of polyethylene terephthalate or the like and have a thickness of 0.00025″ (0.00635 mm) or less.

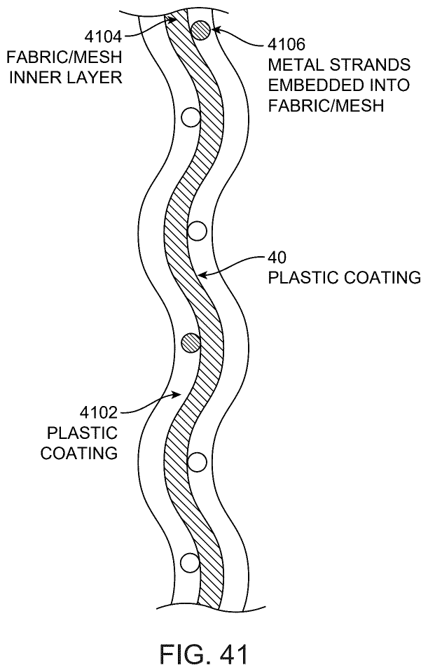

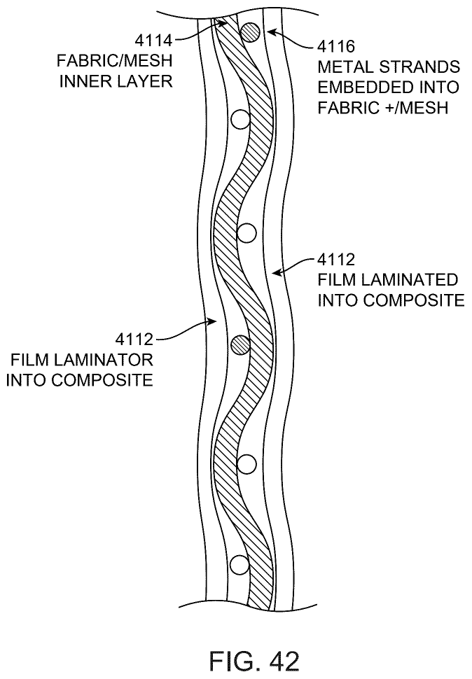

In some cases, as shown in FIG. 41, a plastic coating 4102 covers a fabric or mesh inner layer 4104. Here, metal strands 4106 may be embedded in the fabric or mesh 4104 and likewise covered by the plastic coating 4102. In some embodiments, as shown in FIG. 42, a film 4112 is laminated into the composite forming the inner layer 4114. When the film 4112 is laminated, the melting temperature of the film 4112 is low enough so that the film 4112 in a liquid or semi-liquid state melts and falls into the crevices and there are no air bubbles.

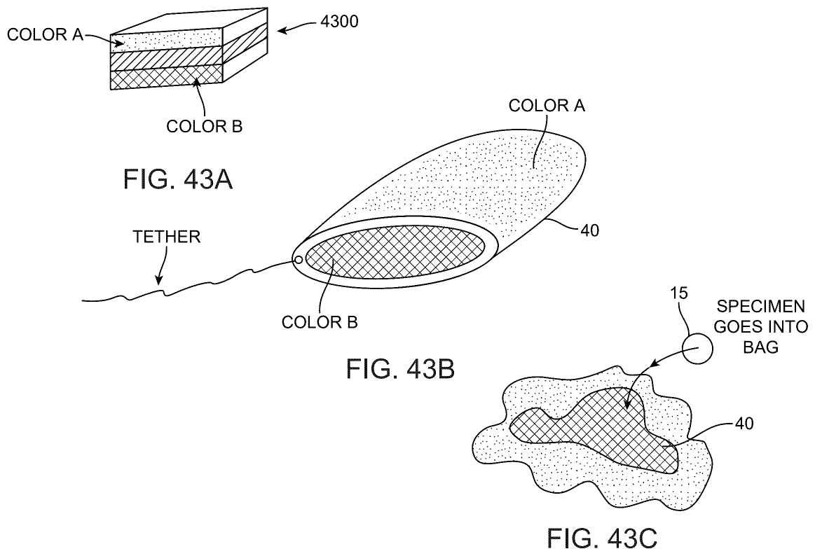

In some embodiments, as shown in FIGS. 43A-43C, the tissue container material 4300 may have multiple colors to distinguish an interior from an exterior of the tissue container 40, to allow a medical practitioner or other user to distinguish the container inside a patient's body cavity 18, e.g., abdomen and make the tissue container 40 easy to spot inside and outside the abdomen of the patient 20. For example, the exterior of the tissue container 40 may have first color (A), e.g., yellow, and the interior may have a second color (B), e.g., green. In other embodiments, some or all of the material may be opaque, translucent, or transparent. Multilayer embodiments may include one or more opaque, translucent, and/or transparent layers.

In some embodiments, the material of the wall 44 of the tissue container 40 may have a parylene coating or related biocompatible coating that provides a strong electrical insulator, for example metalized mylar created with vapor deposition. This slipperiness of the surface of the tissue container 40 using this material enables tissue 15 within the interior volume 42 of the tissue container 40 to spin during cutting. In other embodiments, the tissue container material may have a hydrophilic coating. In other embodiments, the tissue container may include a pre-lubricated surface with suitable lubricant.

In some embodiments, as shown in FIGS. 44A-44F, a balloon 4402, for example, constructed and arranged as an inflatable strip or mechanism or the like along an interior and/or exterior surface of the body of the tissue container 40, can be used help open or unfurl the tissue container 40 of a tissue containment and removal system 4400, for example, in a laparoscopic procedure. After the tissue container 40 has been inserted into the abdomen 18, it is important for the tissue container mouth at the rim 41 and/or the tissue container body to be opened to facilitate placing a tissue specimen 15 into the interior volume 42 of the tissue container 40. Additionally, storage of a loaded tissue container for an extended period of time may, in some cases, cause the material of the tissue container 40 to take a shape or set, making it even more desirable to have an active method of straightening or flattening the tissue container 40 prior to insertion of a tissue specimen 15.

Resilient materials such as certain metals may be used as a spring to provide rigidity to the rim 41 of the tissue container 40, however the rim 41 may also be stiffened through an inflatable component, for example, a balloon or the like forming part of the rim 41. Additionally, the balloon 4402 constructed and arranged as an inflatable strip on the outside of the tissue container 40 or either side of the tissue container 40 may be used to unfurl the container 40 either partially or fully as the user desires. The inflatable strip 4402 could be inflated using a liquid, such as a saline, but may also be inflated with a gas such as air or nitrogen. An external source of pressurized fluid may be coupled to the proximal end of the handle to inflate the strip 4402 via the inflation tube 4403 which is disposed in the handle 4404 and which is used to inflate the strip 4402. It should be noted that the inflation tube 4403 may also be operatively and optionally coupled to a source of negative pressure or neutral pressure in order to control deflation of the balloon 4402, as may be desired prior to removing the tissue container 40 from the patient's body cavity 18. In some instances, the balloon 4402 may be configured such that when a source of negative pressure is operatively coupled to the interior volume of the balloon 4402, the balloon 4402 furls or otherwise contracts the overall profile of the tissue container in order to facilitate removal of the tissue container from the patient's body cavity 18 through the opening 24.

The inflatable strip 4402 or mechanism disposed down the side of the tissue container 40 may be connected to an inflatable hoop or rim 41 or separate from the hoop or rim 41. The tube 4403 of the inflation mechanism, e.g., extending through a lumen of the container handle 4404, may be contained within the handle/ridged member 4404 or can be outside of the handle 4404 or the ridged member. Additionally, the inflatable strip 4402 could take the form of a manifold having multiple balloon manifold sections 4401 extending along a length of the tissue container 40 used to unfurl the tissue container 40 as shown in FIG. 44C. The plurality of inflatable elements 4401 may extend from a main inflatable strip 4402 for some embodiments. Various other patterns or configurations of the multiple manifold sections 4401 may also be used. For example, the main inflatable strip 4402 may be inside the rim 41, or along an edge of the main body of the tissue container 40 parallel the rim 41. FIG. 44D illustrates a configuration wherein an inflatable strip 4501 is configured in a serpentine or sinusoidal pattern in the wall 44 of the tissue container 40 and disposed about the surface of the body of the tissue container 40. For the embodiment shown, the opposed apices of the serpentine pattern of the inflatable strip 4501 are disposed at opposite ends of the tissue container 40. FIG. 44E illustrates a tissue container embodiment 40 similar to that of FIG. 44B but with the inflatable strip 4401 disposed on an outer surface of the tissue container 40. FIG. 44F illustrates a tissue container embodiment 40 similar to that of FIG. 44A but with the inflatable strip 4401 disposed on an outer surface of the tissue container 40.

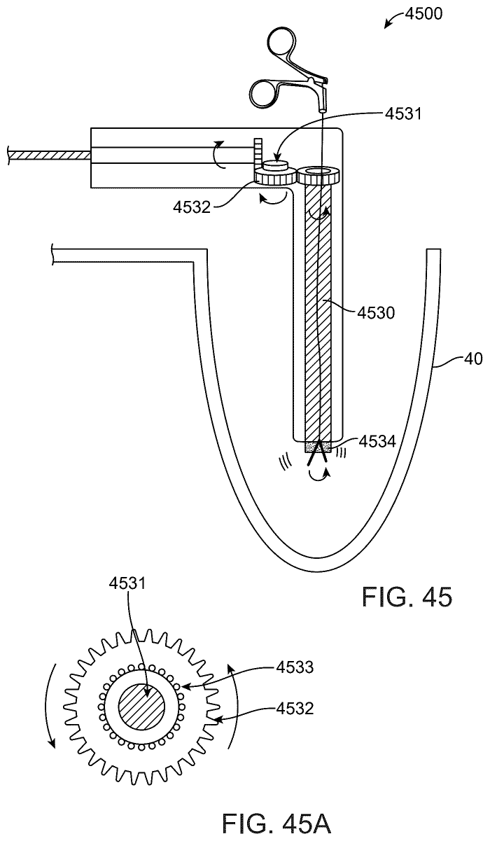

An embodiment of a tissue containment and removal system 4500 is shown in FIGS. 45 and 45A. For this embodiment, an ultrasound energy source may be applied to the cutting blade 4534 of a morcellator 4530 to help facilitate cutting of fibroids of a tissue specimen. The ultrasound energy may be applied in an oscillating fashion and may also be applied to a cutter 4530 having a circular shaped cross section or a square shaped cross section. For the embodiment shown in FIGS. 45 and 45A, an ultrasonic transducer 4531 may be disposed on a drive gear 4532 of the bulk tissue reducer 4530 and configured to transmit ultrasonic energy to the blade tip 4534. In some cases, the drive gear 4532 may rotate on a bearing 4533 and be made of a high strength material such as a metal. In some embodiments, radiofrequency (RF) energy may also be similarly applied to the morcellator 4530 from a source of RF energy either in place of the ultrasound energy or in addition to it.

In embodiments where the tissue cutting modality includes ultrasound energy or radio frequency spectrum energy, or in embodiments wherein other non-rotational cutting modalities are used, the console 60 may not require a drive box 50. For example, embodiments herein that include a vacuum system with no tenaculum 45 as shown in FIGS. 19 and 20 may require a different embodiment of the console 60, e.g., with no drive box 50. Here, features and components of the console 60 described herein may be located in the handle of the cutting device 30, e.g., a morcellator, and communicate with the console 60 via wireless and/or electrically conductive (wired) connectors or the like. Other than the alternative tissue cutting energy used, the system embodiment illustrated in FIGS. 45 and 45A may otherwise be used in any suitable tissue containment and removal method embodiment discussed herein.

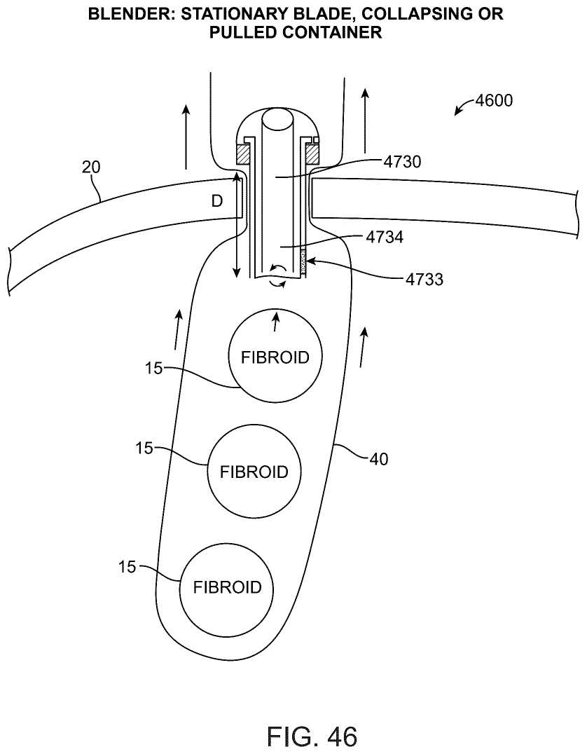

In another tissue containment and removal system embodiment 4600 shown in FIG. 46, the tissue cutter 4730 may be configured to function and morcellate tissue in a manner similar to that of a blender. In some cases, the morcellator blade 4734 can be just barely exposed inside the interior of the tissue container 40 within the abdomen while the container 40 collapses towards the blade 4734 either through mechanical pulling or other mechanism as the blade 4734 continues to cut the tissue specimen 15. In some cases, a distance D between and outer surface or skin of the patient's body 20 and a distal end of the tissue cutter 4730 may be a fixed distance that extends past an inside surface of the abdominal wall 20. For some embodiments, the blade 4734 may be stationary with respect to the abdominal wall. A canula 4733 may be disposed about the tissue cutter 4730 in some instances.

In sum, embodiments herein describe a multi-layer bag with some conductive and other non-conductive elements on a deployable handle/introducer, with an inflatable mechanism to unfold it after introduction into the abdomen which is electrically connected with a tissue reducing device through a console which can determine if the tissue reducing mechanism has contacted the multi-layer material of the container for the purposes of tissue removal in laparoscopic surgery.

Features described herein with respect to different methods of use or different features, instruments, components, or their order of use may interchangeably be used among the various methods without taking away from the spirit of the methods and devices of the present disclosure. The presence or absence of a particular step or component should not be construed as limiting the methods described herein,

With regard to the above detailed description, like reference numerals used therein may refer to like elements that may have the same or similar dimensions, materials and configurations. While particular forms of embodiments have been illustrated and described, it will be apparent that various modifications can be made without departing from the spirit and scope of the embodiments discussed. Accordingly, it is not intended that the invention be limited by the foregoing detailed description.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” may refer to one of or a plurality of the elements it modifies unless it is contextually clear either one of the elements or more than one of the elements is described. Although the present technology has been specifically disclosed by representative embodiments and optional features, modification and variation of the concepts herein disclosed may be made, and such modifications and variations may be considered within the scope of this technology.

Certain embodiments of the technology are set forth in the claims that follow.

What is claimed is:

1. A tissue containment and removal system, comprising:
a tissue container comprising:
an opening;
a multi-layer body that includes a combination of conductive material and non-conductive material forming an interior volume for receiving a tissue specimen through the opening; and
a conductive connector coupled to the conductive material; and
a cable extending between the conductive connector of the tissue container and a console for providing an electrical communication between the conductive material of the tissue container and the console, the cable having at least one elongate core having a structure that bears a tensile load for an electrode, the electrode forming a conductive path for the electrical communication with the conductive material of the multi-layer body.

2. The tissue containment and removal system of claim 1, wherein the conductive connector comprises:
an electrical terminal comprising one end having a crimp barrel for electrically coupled to the cable and another end having an electrical terminal electrically coupled to an edge or rim of the tissue container;
a washer that penetrates a laminate material of the tissue container to provide an electrical connection between the conductive material and the electrical terminal; and
an eyelet or grommet extending through the washer for holding the washer in place between the multi-layer body and the edge or rim of the tissue container.

3. The tissue containment and removal system of claim 1, wherein the body of the multi-layer tissue container includes a composite mesh structure comprised of at least one strand of the conductive material and strands of the non-conductive material.

4. The tissue containment and removal system of claim 1, wherein the cable includes two pins, each pin including the electrode formed of plurality of conductive strands surrounded by an insulative sheath.

5. The tissue containment and removal system of claim 4, wherein the plurality of conductive strands of the electrode wound helically about the elongate core such that initial high loads untwist the conductive strands first, allowing a predetermined amount of elasticity with the electrode, and transferring a tensile load to the at least one elongate core from the electrode.

6. The tissue containment and removal system of claim 1, further comprising the console, wherein the console facilitates an exchange of power and control signals between the tissue container and a medical instrument.

7. The tissue containment and removal system of claim 6, wherein the medical instrument includes a tissue cutter having a tissue cutting blade that is configured to engage with the source of tissue inside the container for the surgical removal of the tissue specimen from the tissue container.

8. The tissue containment and removal system of claim 6, wherein the control signals facilitated between the console, the tissue container, and the medical instrument include an auto-shutoff that disables the medical instrument when the medical instrument forms an electrical path with the conductive material of the multi-layer body of the tissue container.

9. The tissue containment and removal system of claim 8, further comprising a controller and a motor, wherein in response to the auto-shutoff, the controller discontinues actuation of the motor and tissue cutting blade whenever the blade is a predetermined distance from the conductive material.

10. The tissue containment and removal system of claim 1, further comprising an insertion apparatus coupled to an edge or rim of the tissue container for inserting the tissue container into the body cavity.

11. The tissue containment and removal system of claim 10, wherein the insertion apparatus is a handle.

12. The tissue containment and removal system of claim 10, wherein the insertion apparatus is inserted through an incision in the body into the body cavity.

13. The tissue containment and removal system of claim 12, wherein the insertion apparatus injects the tissue container in a closed, deflated, wrapped, or undeployed state extending through one of the ports into the body cavity.

14. The tissue containment and removal system of claim 1, wherein the tissue container further comprises at least one inflation strip coupled to the body for transitioning the tissue container from the closed, deflated, wrapped, or undeployed state to an open state inside the body cavity.

15. The tissue containment and removal system of claim 1, further comprising a microprocessor to determine if the tissue container is an approved container or counterfeit.

16. A tissue containment and removal system, comprising:
a tissue container comprising:
an opening;
a multi-layer body that includes both conductive material and non-conductive material forming an interior volume for receiving a tissue specimen through the opening; and
a tissue cutter, wherein the tissue container is constructed and arranged to receive the tissue cutter through the opening of the multi-layer body about the tissue specimen, the tissue cutter having a tissue cutting blade that is configured to engage with a source of tissue inside the container, and wherein the tissue cutter is automatically inactivated when the tissue cutter forms an electrical path with the conductive material of the multi-layer body of the tissue container.

17. The tissue containment and removal system of claim 16, further comprising a first port for receiving and providing power to a drive cable of the tissue cutter and a second port for receiving and providing power to a conductive tether coupled to the tissue container.

18. The tissue containment and removal system of claim 16, wherein the tether has at least one elongate core having a structure that bears a tensile load for an electrode about the elongate core, the electrode forming a conductive path for the power received from the second port of the console.

19. A tissue removal system, comprising:
a tissue container comprising:
an opening;
a multi-layer body that includes both conductive material and non-conductive material forming an interior volume for receiving a tissue specimen through the opening;
a tissue cutter having a proximal end, a distal end, and a tube extending from the proximal end to the distal end, the tissue cutter including a tissue cutting blade at the distal end that is configured to engage with a tissue specimen inside the container; and
a pressure or vacuum source at the proximal end for causing a change in pressure or force in an interior volume of the tube so that the tissue specimen is drawn toward the tissue cutting blade and into the interior volume of the tube.

20. The tissue removal system of claim 19, further comprising a one-way fluid path from the tissue cutting blade to an opening of the tube at the proximal end.

21. The tissue removal system of claim 19, further comprising a pump at the proximal end for generating a fluid path through a first fluid path in the cutter along an exterior of the tube and through an opening in the tube at the distal end to the interior volume of the tube to an opening of the tube at the proximal end.

22. The tissue removal system of claim 21, further comprising:
a tissue container for receiving the tissue cutter through an opening at the distal end to body that surrounds the tissue specimen through the opening;
a console;
a cable extending between the tissue container and the console for providing a source of electricity to the tissue container; and
a drive cable for providing power to the tissue cutter pressure or vacuum source.

23. The tissue removal system of claim 21, further comprising:
a tissue trap at the proximal end of the tissue cutter that receives and holds the tissue specimen from the tube, wherein the pump drives the tissue specimen from the tube to the tissue trap.

24. The tissue containment and removal system of claim 22, wherein the console has a port for receiving the cable from the container so that the container can detect if the tissue cutter comes into contact with the container and immediately stop the operation of the system.

25. The tissue removal system of claim 19, wherein the tissue cutter, comprises:
a handpiece;
an anti-rotation tube coupled to the handpiece, the anti-rotation tube including a friction-inducing lumen;
an elongate tube having an inner lumen extending about a length of the anti-rotation tube; and
a tissue cutting blade comprising a sharpened distal end of the elongate tube to engage and cut a tissue specimen into at least one tissue component for entry to the friction-inducing lumen of the anti-rotation tube, the tissue cutter configured to rotate about a longitudinal axis thereof with respect to the anti-rotation tube.

26. A multi-layer bag with conductive and non-conductive elements on a deployable handle, comprising: an inflatable mechanism to unfold it after introduction into an abdomen which is electrically connected with a tissue reducing device to a console which can determine if the tissue reducing mechanism has contacted the multi-layer material of the container for the purposes of tissue removal in laparoscopic surgery.
